SEQUENCE LISTING (454 sequences)

SEQ ID NO: 1 - DNA, 55 bp, synthetic construct
  misc_feature 1..55: note = Adaptor
  misc_difference 11..22: note = n is a or c or g or t
  modified_base 55: mod_base = OTHER; note = c is dideoxycytosine
  source 1..55: mol_type = other DNA
ggactccaatnnnnnnnnnnnnacgctaagaaagatcggaagagcacacgtctgc

SEQ ID NO: 2 - DNA, 11 bp, synthetic construct
  misc_feature 1..11: note = adaptor
  source 1..11: mol_type = other DNA
attggagtcct

SEQ ID NO: 3 - DNA, 62 bp, synthetic construct
  misc_feature 1..62: note = Oligonucleotide
  misc_difference 30..39: note = n is a or c or g or t
  misc_difference 61: note = n is a or c or g or t
  misc_difference 62: note = v is a or c or g
  source 1..62: mol_type = other DNA
cgactcactatagggctggaattctgacgnnnnnnnnnnacgttttttttttttttttttnv

SEQ ID NO: 4 - DNA, 56 bp, synthetic construct
  source 1..56: mol_type = other DNA
  misc_feature 1..56: note = Oligonucleotide
  variation 38..47: note = n is a or c or g or t
  modified_base 1: mod_base = OTHER; note = c is 5-methyldeoxyisocytidine
  modified_base 54..56: mod_base = OTHER; note = g is riboguanosine
  modified_base 2..3: mod_base = OTHER; note = g is deoxyisoguanosine
cggtaatacgactcactatagggctggaattctgacgnnnnnnnnnnatctgcggg

SEQ ID NO: 5 - DNA, 98 bp, synthetic construct
  misc_feature 1..98: note = Primer
  source 1..98: mol_type = other DNA
agcagtggtatcaacgcagagtcaagcagaagacggcatacgagattccgaaacgtgactggagttcagacgtgtgctcttccgatctttcttagcgt

SEQ ID NO: 6 - DNA, 115 bp, synthetic construct
  misc_feature 1..115: note = Primer
  source 1..115: mol_type = other DNA
gtgagtgatggttgaggatgtgtgcaagcagaagacggcatacgagattacgtacggtgactggagttcagacgtgtgctcttccgatctcgactcactatagggctggaattct

SEQ ID NO: 7 - DNA, 84 bp, synthetic construct
  misc_feature 1..84: note = Primer
  misc_difference 59..63: note = n is a or c or g or t
  source 1..84: mol_type = other DNA
aatgatacggcgaccaccgagatctacactctttccctacacgacgctcttccgatctnnnnnaatgtacagtattgcgttttg

SEQ ID NO: 8 - DNA, 23 bp, synthetic construct
  misc_feature 1..23: note = Primer
  source 1..23: mol_type = other DNA
aagcagtggtatcaacgcagagt

SEQ ID NO: 9 - DNA, 24 bp, synthetic construct
  misc_feature 1..24: note = Primer
  source 1..24: mol_type = other DNA
gtgagtgatggttgaggatgtgtg

SEQ ID NO: 10 - DNA, 47 bp, synthetic construct
  misc_feature 1..47: note = primer
  source 1..47: mol_type = other DNA
aatgtacagtattgcgttttgagccccaagtcctatgagaacctctg

SEQ ID NO: 11 - DNA, 46 bp, synthetic construct
  misc_feature 1..46: note = primer
  source 1..46: mol_type = other DNA
aatgtacagtattgcgttttgtggcaccagcgatcaggtcctttat

SEQ ID NO: 12 - DNA, 47 bp, synthetic construct
  misc_feature 1..47: note = Primer
  source 1..47: mol_type = other DNA
aatgtacagtattgcgttttgctgagtggagtcacagcggagatagt

SEQ ID NO: 13 - DNA, 51 bp, synthetic construct
  misc_feature 1..51: note = Primer
  source 1..51: mol_type = other DNA
aatgtacagtattgcgttttgtgttccaccagtaacaacagttgaatgtcc

SEQ ID NO: 14 - DNA, 51 bp, synthetic construct
  misc_feature 1..51: note = Primer
  source 1..51: mol_type = other DNA
aatgtacagtattgcgttttggtgtgaggaacatactagtgctttgcaagt

SEQ ID NO: 15 - DNA, 50 bp, synthetic construct
  misc_feature 1..50: note = Primer
  source 1..50: mol_type = other DNA
aatgtacagtattgcgttttgttcaaagttgggtctgcttcagtccaaag

SEQ ID NO: 16 - DNA, 48 bp, synthetic construct
  misc_feature 1..48: note = Primer
  source 1..48: mol_type = other DNA
aatgtacagtattgcgttttgcccccagcttcttctctctgcactaag

SEQ ID NO: 17 - DNA, 51 bp, synthetic construct
  misc_feature 1..51: note = Primer
  source 1..51: mol_type = other DNA
aatgtacagtattgcgttttggccttcccaacatgcattctaacttcttcc

SEQ ID NO: 18 - DNA, 51 bp, synthetic construct
  misc_feature 1..51: note = Primer
  source 1..51: mol_type = other DNA
aatgtacagtattgcgttttgccagctactctcaaaatcagcatcctttgg

SEQ ID NO: 19 - DNA, 51 bp, synthetic construct
  misc_feature 1..51: note = Primer
  source 1..51: mol_type = other DNA
aatgtacagtattgcgttttgccagtccttctgtgagtctatcctcagttc

SEQ ID NO: 20 - DNA, 49 bp, synthetic construct
  misc_feature 1..49: note = Primer
  source 1..49: mol_type = other DNA
aatgtacagtattgcgttttgagagcgaaccaagaatgcctgtttacag

SEQ ID NO: 21 - DNA, 50 bp, synthetic construct
  misc_feature 1..50: note = Primer
  source 1..50: mol_type = other DNA
aatgtacagtattgcgttttggagaggcacgagaacacacatctattctg

SEQ ID NO: 22 - DNA, 50 bp, synthetic construct
  misc_feature 1..50: note = Primer
  source 1..50: mol_type = other DNA
aatgtacagtattgcgttttgttctcttcagaagttccttcgtcatcctt

SEQ ID NO: 23 - DNA, 48 bp, synthetic construct
  misc_feature 1..48: note = Primer
  source 1..48: mol_type = other DNA
aatgtacagtattgcgttttgtgatgacatgccccatcactaaaacac

SEQ ID NO: 24 - DNA, 56 bp, synthetic construct
  misc_feature 1..56: note = Primer
  source 1..56: mol_type = other DNA
aatgtacagtattgcgttttgtgatagagacatgatgtaaccgtgggaatttcttc

SEQ ID NO: 25 - DNA, 55 bp, synthetic construct
  misc_feature 1..55: note = Primer
  source 1..55: mol_type = other DNA
aatgtacagtattgcgttttgcgttctaagagagtgacagaaaggtaaagaggag

SEQ ID NO: 26 - DNA, 57 bp, synthetic construct
  misc_feature 1..57: note = Primer
  source 1..57: mol_type = other DNA
aatgtacagtattgcgttttgatcacaaagtatctttttctgtggcttagaaatctt

SEQ ID NO: 27 - DNA, 57 bp, synthetic construct
  misc_feature 1..57: note = Primer
  source 1..57: mol_type = other DNA
aatgtacagtattgcgttttgtcaaatgttagctcatttttgttaatggtggctttt

SEQ ID NO: 28 - DNA, 57 bp, synthetic construct
  misc_feature 1..57: note = Primer
  source 1..57: mol_type = other DNA
aatgtacagtattgcgttttgtgtcacattataaagattcaggcaatgtttgttagt

SEQ ID NO: 29 - DNA, 57 bp, synthetic construct
  misc_feature 1..57: note = Primer
  source 1..57: mol_type = other DNA
aatgtacagtattgcgttttgagtttgtatgcaacatttctaaagttacctacttgt

SEQ ID NO: 30 - DNA, 57 bp, synthetic construct
  misc_feature 1..57: note = Primer
  source 1..57: mol_type = other DNA
aatgtacagtattgcgttttgaaaatctgttttccaataaattctcagatccaggaa

SEQ ID NO: 31 - DNA, 57 bp, synthetic construct
  misc_feature 1..57: note = Primer
  source 1..57: mol_type = other DNA
aatgtacagtattgcgttttgcgacccagttaccatagcaatttagtgaaataacta

SEQ ID NO: 32 - DNA, 57 bp, synthetic construct
  misc_feature 1..57: note = Primer
  source 1..57: mol_type = other DNA
aatgtacagtattgcgttttgagaggcgctatgtgtattattatagctacctgttaa

SEQ ID NO: 33 - DNA, 55 bp, synthetic construct
  misc_feature 1..55: note = Primer
  source 1..55: mol_type = other DNA
aatgtacagtattgcgttttgcgtttttgacagtttgacagttaaaggcatttcc

SEQ ID NO: 34 - DNA, 57 bp, synthetic construct
  misc_feature 1..57: note = Primer
  source 1..57: mol_type = other DNA
aatgtacagtattgcgttttgctgtccttattttggatatttctcccaatgaaagta

SEQ ID NO: 35 - DNA, 57 bp, synthetic construct
  misc_feature 1..57: note = Primer
  source 1..57: mol_type = other DNA
aatgtacagtattgcgttttggactttttgcaaatgtttaacataggtgacagattt

SEQ ID NO: 36 - DNA, 57 bp, synthetic construct
  misc_feature 1..57: note = Primer
  source 1..57: mol_type = other DNA
aatgtacagtattgcgttttgaagtagaaaatggaagtctatgtgatcaagaaatcg

SEQ ID NO: 37 - DNA, 55 bp, synthetic construct
  misc_feature 1..55: note = Primer
  source 1..55: mol_type = other DNA
aatgtacagtattgcgttttgggcctcttaaagatcatgtttgttacagtgctta

SEQ ID NO: 38 - DNA, 57 bp, synthetic construct
  misc_feature 1..57: note = Primer
  source 1..57: mol_type = other DNA
aatgtacagtattgcgttttgacaagattggtcaggaaaagagaattgttcctataa

SEQ ID NO: 39 - DNA, 57 bp, synthetic construct
  misc_feature 1..57: note = Primer
  source 1..57: mol_type = other DNA
aatgtacagtattgcgttttgagaccctgtctcaaaagtaaaaagtaagttaacatg

SEQ ID NO: 40 - DNA, 56 bp, synthetic construct
  misc_feature 1..56: note = Primer
  source 1..56: mol_type = other DNA
aatgtacagtattgcgttttgtcagtgtcttccaaatccttatgtatagcagcaat

SEQ ID NO: 41 - DNA, 48 bp, synthetic construct
  misc_feature 1..48: note = Primer
  source 1..48: mol_type = other DNA
aatgtacagtattgcgttttgagggtcgaggaagccagtttacatcaa

SEQ ID NO: 42 - DNA, 57 bp, synthetic construct
  misc_feature 1..57: note = Primer
  source 1..57: mol_type = other DNA
aatgtacagtattgcgttttgaacaaaaagatattttcaatatttctgcgcaggttt

SEQ ID NO: 43 - DNA, 57 bp, synthetic construct
  misc_feature 1..57: note = primer
  source 1..57: mol_type = other DNA
aatgtacagtattgcgttttggtctcgacttgaattgcaaaaagatgttagaaaagc

SEQ ID NO: 44 - DNA, 57 bp, synthetic construct
  misc_feature 1..57: note = Primer
  source 1..57: mol_type = other DNA
aatgtacagtattgcgttttgaaaatgttggcagtcataacatttgaaactaatgga

SEQ ID NO: 45 - DNA, 55 bp, synthetic construct
  misc_feature 1..55: note = Primer
  source 1..55: mol_type = other DNA
aatgtacagtattgcgttttgagcctcaaacaggttggttttaaatttgaagtct

SEQ ID NO: 46 - DNA, 56 bp, synthetic construct
  misc_feature 1..56: note = Primer
  source 1..56: mol_type = other DNA
aatgtacagtattgcgttttgcctctgtgtgtatgttttaactacaaagcgaaaca

SEQ ID NO: 47 - DNA, 57 bp, synthetic construct
  misc_feature 1..57: note = Primer
  source 1..57: mol_type = other DNA
aatgtacagtattgcgttttggattcacctggtaatgaggaaaacagctttaaaatc

SEQ ID NO: 48 - DNA, 56 bp, synthetic construct
  misc_feature 1..56: note = Primer
  source 1..56: mol_type = other DNA
aatgtacagtattgcgttttgagatctgctgaaaagaaatttgttaaagcacaatt

SEQ ID NO: 49 - DNA, 45 bp, synthetic construct
  misc_feature 1..45: note = Primer
  source 1..45: mol_type = other DNA
aatgtacagtattgcgttttgcggcatcccctacatcgagacctc

SEQ ID NO: 50 - DNA, 46 bp, synthetic construct
  misc_feature 1..46: note = Primer
  source 1..46: mol_type = other DNA
aatgtacagtattgcgttttgcagggagcagatcaaacgggtgaag

SEQ ID NO: 51 - DNA, 51 bp, synthetic construct
  misc_feature 1..51: note = Primer
  source 1..51: mol_type = other DNA
aatgtacagtattgcgttttgcaagtcttttgaggacatccaccagtacag

SEQ ID NO: 52 - DNA, 46 bp, synthetic construct
  misc_feature 1..46: note = Primer
  source 1..46: mol_type = other DNA
aatgtacagtattgcgttttgacgtgcctgttggacatcctggata

SEQ ID NO: 53 - DNA, 50 bp, synthetic construct
  misc_feature 1..50: note = Primer
  source 1..50: mol_type = other DNA
aatgtacagtattgcgttttgcctgtactggtggatgtcctcaaaagact

SEQ ID NO: 54 - DNA, 49 bp, synthetic construct
  misc_feature 1..49: note = Primer
  source 1..49: mol_type = other DNA
aatgtacagtattgcgttttgccctgaggagcgatgacggaatataagc

SEQ ID NO: 55 - DNA, 52 bp, synthetic construct
  misc_feature 1..52: note = primer
  source 1..52: mol_type = other DNA
aatgtacagtattgcgttttggtcgtattcgtccacaaaatggttctggatc

SEQ ID NO: 56 - DNA, 50 bp, synthetic construct
  misc_feature 1..50: note = Primer
  source 1..50: mol_type = other DNA
aatgtacagtattgcgttttgtgactggcaattgtgtcaacaggtgaaaa

SEQ ID NO: 57 - DNA, 46 bp, synthetic construct
  misc_feature 1..46: note = Primer
  source 1..46: mol_type = other DNA
aatgtacagtattgcgttttgcgccagctggagtttggtcatgttt

SEQ ID NO: 58 - DNA, 57 bp, synthetic construct
  misc_feature 1..57: note = Primer
  source 1..57: mol_type = other DNA
aatgtacagtattgcgttttgaatccctctcatcacaatttcattccacaatagttt

SEQ ID NO: 59 - DNA, 56 bp, synthetic construct
  misc_feature 1..56: note = Primer
  source 1..56: mol_type = other DNA
aatgtacagtattgcgttttgtcaacaacaaagagaatcatgaaatcaaccctagc

SEQ ID NO: 60 - DNA, 46 bp, synthetic construct
  misc_feature 1..46: note = Primer
  source 1..46: mol_type = other DNA
aatgtacagtattgcgttttggatatggagccagcgtgttccgatt

SEQ ID NO: 61 - DNA, 43 bp, synthetic construct
  misc_feature 1..43: note = Primer
  source 1..43: mol_type = other DNA
aatgtacagtattgcgttttgggcgcggaaagtcctcactctc

SEQ ID NO: 62 - DNA, 48 bp, synthetic construct
  misc_feature 1..48: note = Primer
  source 1..48: mol_type = other DNA
aatgtacagtattgcgttttgtatggtgaggttcggcgtgtttaaacg

SEQ ID NO: 63 - DNA, 48 bp, synthetic construct
  misc_feature 1..48: note = Primer
  source 1..48: mol_type = other DNA
aatgtacagtattgcgttttgtggtgacaaagttagaagggtccatgg

SEQ ID NO: 64 - DNA, 50 bp, synthetic construct
  misc_feature 1..50: note = Primer
  source 1..50: mol_type = other DNA
aatgtacagtattgcgttttgcttctttaccaccccagatacgacgacta

SEQ ID NO: 65 - DNA, 45 bp, synthetic construct
  misc_feature 1..45: note = Primer
  source 1..45: mol_type = other DNA
aatgtacagtattgcgttttgcgctcgtggtggtagtcgtcgtat

SEQ ID NO: 66 - DNA, 47 bp, synthetic construct
  misc_feature 1..47: note = Primer
  source 1..47: mol_type = other DNA
aatgtacagtattgcgttttgccaggaggccctttctgtttacaacc

SEQ ID NO: 67 - DNA, 53 bp, synthetic construct
  misc_feature 1..53: note = Primer
  source 1..53: mol_type = other DNA
aatgtacagtattgcgttttgcccacaagcccaaaatattctactcactttgc

SEQ ID NO: 68 - DNA, 49 bp, synthetic construct
  misc_feature 1..49: note = Primer
  source 1..49: mol_type = other DNA
aatgtacagtattgcgttttgatcgcctgcatcaaggaaaaggtaatgg

SEQ ID NO: 69 - DNA, 50 bp, synthetic construct
  misc_feature 1..50: note = Primer
  source 1..50: mol_type = other DNA
aatgtacagtattgcgttttgcgcgtaaggatagcaactgaggttatcac

SEQ ID NO: 70 - DNA, 47 bp, synthetic construct
  misc_feature 1..47: note = Primer
  source 1..47: mol_type = other DNA
aatgtacagtattgcgttttgcgacctgacgtaaccccttgcttatc

SEQ ID NO: 71 - DNA, 51 bp, synthetic construct
  misc_feature 1..51: note = Primer
  source 1..51: mol_type = other DNA
aatgtacagtattgcgttttgggaaatgctctcacgtagtctctcatgtct

SEQ ID NO: 72 - DNA, 51 bp, synthetic construct
  misc_feature 1..51: note = Primer
  source 1..51: mol_type = other DNA
aatgtacagtattgcgttttggtcataacccgaagaacaatgttgccacta

SEQ ID NO: 73 - DNA, 50 bp, synthetic construct
  misc_feature 1..50: note = Primer
  source 1..50: mol_type = other DNA
aatgtacagtattgcgttttggtcagctcaggataaagcacggatggata

SEQ ID NO: 74 - DNA, 57 bp, synthetic construct
  misc_feature 1..57: note = Primer
  source 1..57: mol_type = other DNA
aatgtacagtattgcgttttgctcaggataaaagcttccttcttaacaagtttttcc

SEQ ID NO: 75 - DNA, 53 bp, synthetic construct
  misc_feature 1..53: note = Primer
  source 1..53: mol_type = other DNA
aatgtacagtattgcgttttgagagattgttcccttgcattgacctctttttc

SEQ ID NO: 76 - DNA, 50 bp, synthetic construct
  misc_feature 1..50: note = Primer
  source 1..50: mol_type = other DNA
aatgtacagtattgcgttttgcccctcacctttggaatttacagtctgaa

SEQ ID NO: 77 - DNA, 56 bp, synthetic construct
  misc_feature 1..56: note = Primer
  source 1..56: mol_type = other DNA
aatgtacagtattgcgttttgtaggttcttcaggtctctacactctcctttaaact

SEQ ID NO: 78 - DNA, 52 bp, synthetic construct
  misc_feature 1..52: note = Primer
  source 1..52: mol_type = other DNA
aatgtacagtattgcgttttggagaaggagtgcaatgccaagattatgatcc

SEQ ID NO: 79 - DNA, 50 bp, synthetic construct
  misc_feature 1..50: note = Primer
  source 1..50: mol_type = other DNA
aatgtacagtattgcgttttggacgttctccattgtattggcagtaacca

SEQ ID NO: 80 - DNA, 57 bp, synthetic construct
  misc_feature 1..57: note = Primer
  source 1..57: mol_type = other DNA
aatgtacagtattgcgttttgcacatctcacaggctctaaaggaattctatatccta

SEQ ID NO: 81 - DNA, 52 bp, synthetic construct
  misc_feature 1..52: note = Primer
  source 1..52: mol_type = other DNA
aatgtacagtattgcgttttggaggcaagaggtgagtagtaccaatactgtc

SEQ ID NO: 82 - DNA, 47 bp, synthetic construct
  misc_feature 1..47: note = Prmer
  source 1..47: mol_type = other DNA
aatgtacagtattgcgttttggagcccctccgcttacttgtaatctg

SEQ ID NO: 83 - DNA, 57 bp, synthetic construct
  misc_feature 1..57: note = Primer
  source 1..57: mol_type = other DNA
aatgtacagtattgcgttttgccagtaaaacgtattgagaaaaaggtaaaagcgtta

SEQ ID NO: 84 - DNA, 57 bp, synthetic construct
  misc_feature 1..57: note = Primer
  source 1..57: mol_type = other DNA
aatgtacagtattgcgttttggctcagaataaatcgtaacaatctcaaagtgcattt

SEQ ID NO: 85 - DNA, 49 bp, synthetic construct
  misc_feature 1..49: note = Primer
  source 1..49: mol_type = other DNA
aatgtacagtattgcgttttgtgaggtgtccacagggctcaatctttac

SEQ ID NO: 86 - DNA, 57 bp, synthetic construct
  misc_feature 1..57: note = Primer
  source 1..57: mol_type = other DNA
aatgtacagtattgcgttttgccccttgtatcagtaaaggctatataataccgaatt

SEQ ID NO: 87 - DNA, 55 bp, synthetic construct
  misc_feature 1..55: note = Primer
  source 1..55: mol_type = other DNA
aatgtacagtattgcgttttgtcatgaagagagtatcatcagctcgttcatcatc

SEQ ID NO: 88 - DNA, 53 bp, synthetic construct
  misc_feature 1..53: note = Primer
  source 1..53: mol_type = other DNA
aatgtacagtattgcgttttgtgtcctttctgccgatgtgaaattaaaggtac

SEQ ID NO: 89 - DNA, 47 bp, synthetic construct
  misc_feature 1..47: note = Primer
  source 1..47: mol_type = other DNA
aatgtacagtattgcgttttgtcgccccaaataatttcctgcgaaca

SEQ ID NO: 90 - DNA, 53 bp, synthetic construct
  misc_feature 1..53: note = Primer
  source 1..53: mol_type = other DNA
aatgtacagtattgcgttttgctcatacctccattccaagctttcattgtctc

SEQ ID NO: 91 - DNA, 52 bp, synthetic construct
  misc_feature 1..52: note = Primer
  source 1..52: mol_type = other DNA
aatgtacagtattgcgttttgcctgcccttatttttaacagcaggaacgaat

SEQ ID NO: 92 - DNA, 48 bp, synthetic construct
  misc_feature 1..48: note = Primer
  source 1..48: mol_type = other DNA
aatgtacagtattgcgttttgtcgatagcgaaagtcctctttggtcag

SEQ ID NO: 93 - DNA, 57 bp, synthetic construct
  misc_feature 1..57: note = Primer
  source 1..57: mol_type = other DNA
aatgtacagtattgcgttttggttaaagaccaaccactaactaagagactttccaag

SEQ ID NO: 94 - DNA, 52 bp, synthetic construct
  misc_feature 1..52: note = Primer
  source 1..52: mol_type = other DNA
aatgtacagtattgcgttttgaaacctcttccagtaccttcttcatggttct

SEQ ID NO: 95 - DNA, 51 bp, synthetic construct
  misc_feature 1..51: note = Primer
  source 1..51: mol_type = other DNA
aatgtacagtattgcgttttgtttccaggtgatgtgctctatgaactcctt

SEQ ID NO: 96 - DNA, 45 bp, synthetic construct
  misc_feature 1..45: note = Primer
  source 1..45: mol_type = other DNA
aatgtacagtattgcgttttgggagcggtgcaacagttcaatggt

SEQ ID NO: 97 - DNA, 48 bp, synthetic construct
  misc_feature 1..48: note = Primer
  source 1..48: mol_type = other DNA
aatgtacagtattgcgttttgcatccgtggataatgtgcaccataacc

SEQ ID NO: 98 - DNA, 47 bp, synthetic construct
  misc_feature 1..47: note = Primer
  source 1..47: mol_type = other DNA
aatgtacagtattgcgttttgtcggagagcctggactgtttgaaatc

SEQ ID NO: 99 - DNA, 46 bp, synthetic construct
  misc_feature 1..46: note = Primer
  source 1..46: mol_type = other DNA
aatgtacagtattgcgttttgaagccaggtcttcccgatgagagag

SEQ ID NO: 100 - DNA, 46 bp, synthetic construct
  misc_feature 1..46: note = Primer
  source 1..46: mol_type = other DNA
aatgtacagtattgcgttttgggcactccgtggatttcaaacagtc

SEQ ID NO: 101 - DNA, 53 bp, synthetic construct
  misc_feature 1..53: note = Primer
  source 1..53: mol_type = other DNA
aatgtacagtattgcgttttgcagatatctgctgcccttttaccttatggttt

SEQ ID NO: 102 - DNA, 54 bp, synthetic construct
  misc_feature 1..54: note = Primer
  source 1..54: mol_type = other DNA
aatgtacagtattgcgttttgtgtagactgctttgggattacgtctatcagttg

SEQ ID NO: 103 - DNA, 52 bp, synthetic construct
  misc_feature 1..52: note = primer
  source 1..52: mol_type = other DNA
aatgtacagtattgcgttttgggaaaggagaaaaaggaagtgctacctgaac

SEQ ID NO: 104 - DNA, 51 bp, synthetic construct
  misc_feature 1..51: note = Primer
  source 1..51: mol_type = other DNA
aatgtacagtattgcgttttgtttttctcccttcctcctttgaacaaacag

SEQ ID NO: 105 - DNA, 55 bp, synthetic construct
  misc_feature 1..55: note = Primer
  source 1..55: mol_type = other DNA
aatgtacagtattgcgttttgacagctttaggaaaatggaatctcttacctcctc

SEQ ID NO: 106 - DNA, 48 bp, synthetic construct
  misc_feature 1..48: note = Primer
  source 1..48: mol_type = other DNA
aatgtacagtattgcgttttggggtgttatggtcgcgttggatttctg

SEQ ID NO: 107 - DNA, 45 bp, synthetic construct
  misc_feature 1..45: note = Primer
  source 1..45: mol_type = other DNA
aatgtacagtattgcgttttggctacggcgtgcaactcacagaac

SEQ ID NO: 108 - DNA, 44 bp, synthetic construct
  misc_feature 1..44: note = Primer
  source 1..44: mol_type = other DNA
aatgtacagtattgcgttttgaccgacctcttccagcgctactt

SEQ ID NO: 109 - DNA, 44 bp, synthetic construct
  misc_feature 1..44: note = Primer
  source 1..44: mol_type = other DNA
aatgtacagtattgcgttttgcgggcagggcttacttaccttgg

SEQ ID NO: 110 - DNA, 49 bp, synthetic construct
  misc_feature 1..49: note = Primer
  source 1..49: mol_type = other DNA
aatgtacagtattgcgttttgtagctactgcctgccttcgaagaacgat

SEQ ID NO: 111 - DNA, 54 bp, synthetic construct
  misc_feature 1..54: note = Primer
  source 1..54: mol_type = other DNA
aatgtacagtattgcgttttgtgtgggtggaaaaagatgtggttaagaaacaac

SEQ ID NO: 112 - DNA, 50 bp, synthetic construct
  misc_feature 1..50: note = Primer
  source 1..50: mol_type = other DNA
aatgtacagtattgcgttttgcccccatatagcttaatctgatgggcatc

SEQ ID NO: 113 - DNA, 51 bp, synthetic construct
  misc_feature 1..51: note = Primer
  source 1..51: mol_type = other DNA
aatgtacagtattgcgttttggaaagagcatcaggaacaagccttgagtac

SEQ ID NO: 114 - DNA, 51 bp, synthetic construct
  misc_feature 1..51: note = Primer
  source 1..51: mol_type = other DNA
aatgtacagtattgcgttttgttgagatgcctgacaacctttacacctttg

SEQ ID NO: 115 - DNA, 49 bp, synthetic construct
  misc_feature 1..49: note = Primer
  source 1..49: mol_type = other DNA
aatgtacagtattgcgttttgctctagggctgagggaatatgcatctct

SEQ ID NO: 116 - DNA, 49 bp, synthetic construct
  misc_feature 1..49: note = Primer
  source 1..49: mol_type = other DNA
aatgtacagtattgcgttttgcgtacccagaagacaatggcctagctat

SEQ ID NO: 117 - DNA, 45 bp, synthetic construct
  misc_feature 1..45: note = Primer
  source 1..45: mol_type = other DNA
aatgtacagtattgcgttttggggcagcacagattcccttaacca

SEQ ID NO: 118 - DNA, 49 bp, synthetic construct
  misc_feature 1..49: note = Primer
  source 1..49: mol_type = other DNA
aatgtacagtattgcgttttgccataccttggctatcccctgaaagttg

SEQ ID NO: 119 - DNA, 46 bp, synthetic construct
  misc_feature 1..46: note = Primer
  source 1..46: mol_type = other DNA
aatgtacagtattgcgttttggccctgatgctcatggagtgttcct

SEQ ID NO: 120 - DNA, 44 bp, synthetic construct
  misc_feature 1..44: note = Primer
  source 1..44: mol_type = other DNA
aatgtacagtattgcgttttgcctggtggttgggagacgactac

SEQ ID NO: 121 - DNA, 49 bp, synthetic construct
  misc_feature 1..49: note = Primer
  source 1..49: mol_type = other DNA
aatgtacagtattgcgttttgtgctgacaggacacagaacaagatacct

SEQ ID NO: 122 - DNA, 52 bp, synthetic construct
  misc_feature 1..52: note = Primer
  source 1..52: mol_type = other DNA
aatgtacagtattgcgttttgggtacaggtatcttgttctgtgtcctgtcag

SEQ ID NO: 123 - DNA, 43 bp, synthetic construct
  misc_feature 1..43: note = Primer
  source 1..43: mol_type = other DNA
aatgtacagtattgcgttttggagtcccgggctcgattcacag

SEQ ID NO: 124 - DNA, 50 bp, synthetic construct
  misc_feature 1..50: note = Primer
  source 1..50: mol_type = other DNA
aatgtacagtattgcgttttgctggtcagagaggtgtgtactgattgtct

SEQ ID NO: 125 - DNA, 57 bp, synthetic construct
  misc_feature 1..57: note = Primer
  source 1..57: mol_type = other DNA
aatgtacagtattgcgttttgaggaaagatcaattacattcacaagttcacacttct

SEQ ID NO: 126 - DNA, 55 bp, synthetic construct
  misc_feature 1..55: note = Primer
  source 1..55: mol_type = other DNA
aatgtacagtattgcgttttgctgcacagttcagaggatatttaagctcaatgac

SEQ ID NO: 127 - DNA, 49 bp, synthetic construct
  misc_feature 1..49: note = Primer
  source 1..49: mol_type = other DNA
aatgtacagtattgcgttttgcacagaccgtcatgcatttctgacactc

SEQ ID NO: 128 - DNA, 47 bp, synthetic construct
  misc_feature 1..47: note = Primer
  source 1..47: mol_type = other DNA
aatgtacagtattgcgttttgaggctggtacctgctcttcttcaatc

SEQ ID NO: 129 - DNA, 53 bp, synthetic construct
  misc_feature 1..53: note = Primer
  source 1..53: mol_type = other DNA
aatgtacagtattgcgttttgcgaaatcaaacagttgtctatcagagcctgtc

SEQ ID NO: 130 - DNA, 55 bp, synthetic construct
  misc_feature 1..55: note = Primer
  source 1..55: mol_type = other DNA
aatgtacagtattgcgttttgacaaaagaaaagaagtcatgtctgtatgtggaaa

SEQ ID NO: 131 - DNA, 57 bp, synthetic construct
  misc_feature 1..57: note = Primer
  source 1..57: mol_type = other DNA
aatgtacagtattgcgttttgtccaggataatacacatcacagtaaataacactctg

SEQ ID NO: 132 - DNA, 55 bp, synthetic construct
  misc_feature 1..55: note = Primer
  source 1..55: mol_type = other DNA
aatgtacagtattgcgttttgcatcctctttgtcatcaagctacagtctttttga

SEQ ID NO: 133 - DNA, 51 bp, synthetic construct
  misc_feature 1..51: note = Primer
  source 1..51: mol_type = other DNA
aatgtacagtattgcgttttgctcccatttttgtgcatctttgttgctgtc

SEQ ID NO: 134 - DNA, 55 bp, synthetic construct
  misc_feature 1..55: note = Primer
  source 1..55: mol_type = other DNA
aatgtacagtattgcgttttgcagaactgcctattcctaactgactcatcatttc

SEQ ID NO: 135 - DNA, 54 bp, synthetic construct
  misc_feature 1..54: note = Primer
  source 1..54: mol_type = other DNA
aatgtacagtattgcgttttggaattctgtttcatcgctgagtgacactctttt

SEQ ID NO: 136 - DNA, 57 bp, synthetic construct
  misc_feature 1..57: note = Primer
  source 1..57: mol_type = other DNA
aatgtacagtattgcgttttgtttttacctttgcttttacctttttgtacttgtgac

SEQ ID NO: 137 - DNA, 53 bp, synthetic construct
  misc_feature 1..53: note = Primer
  source 1..53: mol_type = other DNA
aatgtacagtattgcgttttgagaaggagtctggaatagaaaggctaacagaa

SEQ ID NO: 138 - DNA, 49 bp, synthetic construct
  misc_feature 1..49: note = Primer
  source 1..49: mol_type = other DNA
aatgtacagtattgcgttttgcacaagatgtgccaagggaattgtatgc

SEQ ID NO: 139 - DNA, 57 bp, synthetic construct
  misc_feature 1..57: note = Primer
  source 1..57: mol_type = other DNA
aatgtacagtattgcgttttgaagagtcaataggtcagagagttttatgttcttcca

SEQ ID NO: 140 - DNA, 52 bp, synthetic construct
  misc_feature 1..52: note = Primer
  source 1..52: mol_type = other DNA
aatgtacagtattgcgttttgactgatcttctcaaagtcgtcatccttcagt

SEQ ID NO: 141 - DNA, 57 bp, synthetic construct
  misc_feature 1..57: note = Primer
  source 1..57: mol_type = other DNA
aatgtacagtattgcgttttgaccctgagaaataatccaattacctgttaatcaagg

SEQ ID NO: 142 - DNA, 57 bp, synthetic construct
  misc_feature 1..57: note = Primer
  source 1..57: mol_type = other DNA
aatgtacagtattgcgttttgaaaaggtattgagtaaaatcagtcttccttctaccc

SEQ ID NO: 143 - DNA, 55 bp, synthetic construct
  misc_feature 1..55: note = Primer
  source 1..55: mol_type = other DNA
aatgtacagtattgcgttttgccttcctccctctttctttcataaaacctctctt

SEQ ID NO: 144 - DNA, 45 bp, synthetic construct
  misc_feature 1..45: note = Primer
  source 1..45: mol_type = other DNA
aatgtacagtattgcgttttggccagagccacccaactcttaagg

SEQ ID NO: 145 - DNA, 57 bp, synthetic construct
  misc_feature 1..57: note = Primer
  source 1..57: mol_type = other DNA
aatgtacagtattgcgttttgtggaagaggaatttaataacgaacgttttaagagga

SEQ ID NO: 146 - DNA, 47 bp, synthetic construct
  misc_feature 1..47: note = Primer
  source 1..47: mol_type = other DNA
aatgtacagtattgcgttttggcatctactgccgaggatgttccaag

SEQ ID NO: 147 - DNA, 46 bp, synthetic construct
  misc_feature 1..46: note = Primer
  source 1..46: mol_type = other DNA
aatgtacagtattgcgttttgcacagtgagctcaagtgcgacatca

SEQ ID NO: 148 - DNA, 43 bp, synthetic construct
  misc_feature 1..43: note = Primer
  source 1..43: mol_type = other DNA
aatgtacagtattgcgttttgccgactggccatctcctcgtag

SEQ ID NO: 149 - DNA, 45 bp, synthetic construct
  misc_feature 1..45: note = Primer
  source 1..45: mol_type = other DNA
aatgtacagtattgcgttttggtaccagcgcgactacgaggagat

SEQ ID NO: 150 - DNA, 57 bp, synthetic construct
  misc_feature 1..57: note = Primer
  source 1..57: mol_type = other DNA
aatgtacagtattgcgttttgtcttttctgtcaaatggagatgatctcttctgactc

SEQ ID NO: 151 - DNA, 48 bp, synthetic construct
  misc_feature 1..48: note = Primer
  source 1..48: mol_type = other DNA
aatgtacagtattgcgttttggggagcccatcatctgcaaaaacatcc

SEQ ID NO: 152 - DNA, 52 bp, synthetic construct
  misc_feature 1..52: note = Primer
  source 1..52: mol_type = other DNA
aatgtacagtattgcgttttgaagctgaagaagatgtggaaaagtcccaatg

SEQ ID NO: 153 - DNA, 47 bp, synthetic construct
  misc_feature 1..47: note = Primer
  source 1..47: mol_type = other DNA
aatgtacagtattgcgttttggcgtgggatgtttttgcagatgatgg

SEQ ID NO: 154 - DNA, 44 bp, synthetic construct
  misc_feature 1..44: note = Primer
  source 1..44: mol_type = other DNA
aatgtacagtattgcgttttgcgacgctgaggacgctatggatg

SEQ ID NO: 155 - DNA, 43 bp, synthetic construct
  misc_feature 1..43: note = Primer
  source 1..43: mol_type = other DNA
aatgtacagtattgcgttttggctgaggcgcgtcttcgagaag

SEQ ID NO: 156 - DNA, 44 bp, synthetic construct
  misc_feature 1..44: note = Primer
  source 1..44: mol_type = other DNA
aatgtacagtattgcgttttggcgcttgtcgtgaaagcgaacga

SEQ ID NO: 157 - DNA, 42 bp, synthetic construct
  misc_feature 1..42: note = Primer
  source 1..42: mol_type = other DNA
aatgtacagtattgcgttttggctgcccgcccagttgttact

SEQ ID NO: 158 - DNA, 51 bp, synthetic construct
  misc_feature 1..51: note = Primer
  source 1..51: mol_type = other DNA
aatgtacagtattgcgttttgagactctggactgatgaagcaattctgagt

SEQ ID NO: 159 - DNA, 48 bp, synthetic construct
  misc_feature 1..48: note = Primer
  source 1..48: mol_type = other DNA
aatgtacagtattgcgttttgtcaccggtgacaccttaaaaccaaagc

SEQ ID NO: 160 - DNA, 50 bp, synthetic construct
  misc_feature 1..50: note = Primer
  source 1..50: mol_type = other DNA
aatgtacagtattgcgttttgggctcctttgtacctcctccatcttgatc

SEQ ID NO: 161 - DNA, 57 bp, synthetic construct
  misc_feature 1..57: note = Primer
  source 1..57: mol_type = other DNA
aatgtacagtattgcgttttggtcagttgtctaacaataacaaagatctgctcttgg

SEQ ID NO: 162 - DNA, 49 bp, synthetic construct
  misc_feature 1..49: note = Primer
  source 1..49: mol_type = other DNA
aatgtacagtattgcgttttgggtgggcagcaagaaaaagtccagtaaa

SEQ ID NO: 163 - DNA, 49 bp, synthetic construct
  misc_feature 1..49: note = Primer
  source 1..49: mol_type = other DNA
aatgtacagtattgcgttttggccaaggctttctctggcatgatctttt

SEQ ID NO: 164 - DNA, 55 bp, synthetic construct
  misc_feature 1..55: note = Primer
  source 1..55: mol_type = other DNA
aatgtacagtattgcgttttgggataactttctcagcatttccaccagtttcaag

SEQ ID NO: 165 - DNA, 57 bp, synthetic construct
  misc_feature 1..57: note = Primer
  source 1..57: mol_type = other DNA
aatgtacagtattgcgttttgtgtccctaagttgagtaaaatgatagagaatgagtc

SEQ ID NO: 166 - DNA, 50 bp, synthetic construct
  misc_feature 1..50: note = Primer
  source 1..50: mol_type = other DNA
aatgtacagtattgcgttttggctgccagaaatccagcatccaaaatttg

SEQ ID NO: 167 - DNA, 51 bp, synthetic construct
  misc_feature 1..51: note = Primer
  source 1..51: mol_type = other DNA
aatgtacagtattgcgttttggtcgctttcttttcttagtgccaggaaact

SEQ ID NO: 168 - DNA, 50 bp, synthetic construct
  misc_feature 1..50: note = Primer
  source 1..50: mol_type = other DNA
aatgtacagtattgcgttttgacagtcgagacgattcatgagggaacttc

SEQ ID NO: 169 - DNA, 48 bp, synthetic construct
  misc_feature 1..48: note = Primer
  source 1..48: mol_type = other DNA
aatgtacagtattgcgttttgggaaagctcggcgtgttggataagaag

SEQ ID NO: 170 - DNA, 48 bp, synthetic construct
  misc_feature 1..48: note = Primer
  source 1..48: mol_type = other DNA
aatgtacagtattgcgttttgacgccacaagtgactgaaagttggaag

SEQ ID NO: 171 - DNA, 50 bp, synthetic construct
  misc_feature 1..50: note = Primer
  source 1..50: mol_type = other DNA
aatgtacagtattgcgttttgtgatgggctggagatttggcatagttttc

SEQ ID NO: 172 - DNA, 50 bp, synthetic construct
  misc_feature 1..50: note = Primer
  source 1..50: mol_type = other DNA
aatgtacagtattgcgttttgctatgcacccactttcaacacagttaggt

SEQ ID NO: 173 - DNA, 47 bp, synthetic construct
  misc_feature 1..47: note = Primer
  source 1..47: mol_type = other DNA
aatgtacagtattgcgttttggcttggtcagaagtgctgttgttgtc

SEQ ID NO: 174 - DNA, 47 bp, synthetic construct
  misc_feature 1..47: note = Primer
  source 1..47: mol_type = other DNA
aatgtacagtattgcgttttgcgtgggccagaaagttgtccacaatg

SEQ ID NO: 175 - DNA, 52 bp, synthetic construct
  misc_feature 1..52: note = Primer
  source 1..52: mol_type = other DNA
aatgtacagtattgcgttttggggatatggattctcgtggtagaaggtgtaa

SEQ ID NO: 176 - DNA, 54 bp, synthetic construct
  misc_feature 1..54: note = Primer
  source 1..54: mol_type = other DNA
aatgtacagtattgcgttttgctaatcaccaagttccaagtgttcagaatctcc

SEQ ID NO: 177 - DNA, 55 bp, synthetic construct
  misc_feature 1..55: note = Primer
  source 1..55: mol_type = other DNA
aatgtacagtattgcgttttgaccgtaataaccaaggttcatcataggcattgat

SEQ ID NO: 178 - DNA, 49 bp, synthetic construct
  misc_feature 1..49: note = Primer
  source 1..49: mol_type = other DNA
aatgtacagtattgcgttttgtcccagtggaagttactatgcaccctat

SEQ ID NO: 179 - DNA, 54 bp, synthetic construct
  misc_feature 1..54: note = Primer
  source 1..54: mol_type = other DNA
aatgtacagtattgcgttttgtgcttatgcttgtgtttgtgtttcctcttatgg

SEQ ID NO: 180 - DNA, 54 bp, synthetic construct
  misc_feature 1..54: note = Primer
  source 1..54: mol_type = other DNA
aatgtacagtattgcgttttggcttctgtttctccttatgcttgttcttctcac

SEQ ID NO: 181 - DNA, 47 bp, synthetic construct
  misc_feature 1..47: note = Primer
  source 1..47: mol_type = other DNA
aatgtacagtattgcgttttgcctgagtggtctttttgcaggcaaag

SEQ ID NO: 182 - DNA, 47 bp, synthetic construct
  misc_feature 1..47: note = Primer
  source 1..47: mol_type = other DNA
aatgtacagtattgcgttttgccggccacaaagcttctaagaacaac

SEQ ID NO: 183 - DNA, 47 bp, synthetic construct
  misc_feature 1..47: note = Primer
  source 1..47: mol_type = other DNA
aatgtacagtattgcgttttggcggttcatcttgaaggcttggatgt

SEQ ID NO: 184 - DNA, 51 bp, synthetic construct
  misc_feature 1..51: note = Primer
  source 1..51: mol_type = other DNA
aatgtacagtattgcgttttgttcagtgaaatgaacccttcgaatgacaag

SEQ ID NO: 185 - DNA, 48 bp, synthetic construct
  misc_feature 1..48: note = Primer
  source 1..48: mol_type = other DNA
aatgtacagtattgcgttttgctcctcctcctctttgcgtttcttgtc

SEQ ID NO: 186 - DNA, 51 bp, synthetic construct
  misc_feature 1..51: note = Primer
  source 1..51: mol_type = other DNA
aatgtacagtattgcgttttggcagcagagaaacaaatgaaggacaaacag

SEQ ID NO: 187 - DNA, 52 bp, synthetic construct
  misc_feature 1..52: note = Primer
  source 1..52: mol_type = other DNA
aatgtacagtattgcgttttgtaaggaggaggaagaagacaagaaacgcaaa

SEQ ID NO: 188 - DNA, 50 bp, synthetic construct
  misc_feature 1..50: note = Primer
  source 1..50: mol_type = other DNA
aatgtacagtattgcgttttgtaaggcaggtctgtgagcacaaaatttgg

SEQ ID NO: 189 - DNA, 45 bp, synthetic construct
  misc_feature 1..45: note = Primer
  source 1..45: mol_type = other DNA
aatgtacagtattgcgttttgtggagctgaccagtgacaatgacc

SEQ ID NO: 190 - DNA, 46 bp, synthetic construct
  misc_feature 1..46: note = Primer
  source 1..46: mol_type = other DNA
aatgtacagtattgcgttttgggccaagaagtcggtggacaagaac

SEQ ID NO: 191 - DNA, 43 bp, synthetic construct
  misc_feature 1..43: note = Primer
  source 1..43: mol_type = other DNA
aatgtacagtattgcgttttggcgcaggcggtcattgtcactg

SEQ ID NO: 192 - DNA, 48 bp, synthetic construct
  misc_feature 1..48: note = Primer
  source 1..48: mol_type = other DNA
aatgtacagtattgcgttttgttgctgttcttgtccaccgacttcttg

SEQ ID NO: 193 - DNA, 42 bp, synthetic construct
  misc_feature 1..42: note = Primer
  source 1..42: mol_type = other DNA
aatgtacagtattgcgttttggcagtgcgcgatctggaactg

SEQ ID NO: 194 - DNA, 42 bp, synthetic construct
  misc_feature 1..42: note = Primer
  source 1..42: mol_type = other DNA
aatgtacagtattgcgttttgcggcggcgactttgactaccc

SEQ ID NO: 195 - DNA, 45 bp, synthetic construct
  misc_feature 1..45: note = Primer
  source 1..45: mol_type = other DNA
aatgtacagtattgcgttttggagcacgagacgtccatcgacatc

SEQ ID NO: 196 - DNA, 43 bp, synthetic construct
  misc_feature 1..43: note = Primer
  source 1..43: mol_type = other DNA
aatgtacagtattgcgttttgcggccaggaactcgtcgttgaa

SEQ ID NO: 197 - DNA, 45 bp, synthetic construct
  misc_feature 1..45: note = Primer
  source 1..45: mol_type = other DNA
aatgtacagtattgcgttttggccatgccgggagaactctaactc

SEQ ID NO: 198 - DNA, 54 bp, synthetic construct
  misc_feature 1..54: note = Primer
  source 1..54: mol_type = other DNA
aatgtacagtattgcgttttgtgtaaccctcctaagtgttcatacgttgtcttg

SEQ ID NO: 199 - DNA, 57 bp, synthetic construct
  misc_feature 1..57: note = Primer
  source 1..57: mol_type = other DNA
aatgtacagtattgcgttttggtcttggtctctgttatatcttgagtctagaacagt

SEQ ID NO: 200 - DNA, 49 bp, synthetic construct
  misc_feature 1..49: note = Primer
  source 1..49: mol_type = other DNA
aatgtacagtattgcgttttgcaggagaacatggaggcgagaagaaaat

SEQ ID NO: 201 - DNA, 48 bp, synthetic construct
  misc_feature 1..48: note = Primer
  source 1..48: mol_type = other DNA
aatgtacagtattgcgttttggggaaagattggatgccgggaatcaac

SEQ ID NO: 202 - DNA, 46 bp, synthetic construct
  misc_feature 1..46: note = Primer
  source 1..46: mol_type = other DNA
aatgtacagtattgcgttttgcggaggcttgattaggtaggaggtg

SEQ ID NO: 203 - DNA, 45 bp, synthetic construct
  misc_feature 1..45: note = Primer
  source 1..45: mol_type = other DNA
aatgtacagtattgcgttttggcggcagctcaacgagaataaaca

SEQ ID NO: 204 - DNA, 45 bp, synthetic construct
  misc_feature 1..45: note = Primer
  source 1..45: mol_type = other DNA
aatgtacagtattgcgttttggcccgcatccttactccgcttatc

SEQ ID NO: 205 - DNA, 49 bp, synthetic construct
  misc_feature 1..49: note = Primer
  source 1..49: mol_type = other DNA
aatgtacagtattgcgttttggctggtttcaaggtaagtggactcttcc

SEQ ID NO: 206 - DNA, 47 bp, synthetic construct
  misc_feature 1..47: note = Primer
  source 1..47: mol_type = other DNA
aatgtacagtattgcgttttggggaatgactgacggagaatcccaac

SEQ ID NO: 207 - DNA, 48 bp, synthetic construct
  misc_feature 1..48: note = Primer
  source 1..48: mol_type = other DNA
aatgtacagtattgcgttttgctaagaccgagagcctgtaggagcttt

SEQ ID NO: 208 - DNA, 42 bp, synthetic construct
  misc_feature 1..42: note = Primer
  source 1..42: mol_type = other DNA
aatgtacagtattgcgttttggccgggcttgtctggtcatct

SEQ ID NO: 209 - DNA, 51 bp, synthetic construct
  misc_feature 1..51: note = Primer
  source 1..51: mol_type = other DNA
aatgtacagtattgcgttttgcagctcacctccaaaaaggcaaaattcttg

SEQ ID NO: 210 - DNA, 48 bp, synthetic construct
  misc_feature 1..48: note = Primer
  source 1..48: mol_type = other DNA
aatgtacagtattgcgttttggcaggaggccatgatggatttcttcaa

SEQ ID NO: 211 - DNA, 53 bp, synthetic construct
  misc_feature 1..53: note = Primer
  source 1..53: mol_type = other DNA
aatgtacagtattgcgttttgcatgagtgaaaggaaagaggaaatcccaatcc

SEQ ID NO: 212 - DNA, 56 bp, synthetic construct
  misc_feature 1..56: note = Primer
  source 1..56: mol_type = other DNA
aatgtacagtattgcgttttgcctatcttccacagtacttacacaacttcctaagc

SEQ ID NO: 213 - DNA, 45 bp, synthetic construct
  misc_feature 1..45: note = Primer
  source 1..45: mol_type = other DNA
aatgtacagtattgcgttttgctcgccgtagactgtccaggtttt

SEQ ID NO: 214 - DNA, 47 bp, synthetic construct
  misc_feature 1..47: note = Primer
  source 1..47: mol_type = other DNA
aatgtacagtattgcgttttgctcacctgatccgtgacgttgatgtc

SEQ ID NO: 215 - DNA, 44 bp, synthetic construct
  misc_feature 1..44: note = Primer
  source 1..44: mol_type = other DNA
aatgtacagtattgcgttttggccctgatggactctcggctact

SEQ ID NO: 216 - DNA, 53 bp, synthetic construct
  misc_feature 1..53: note = Primer
  source 1..53: mol_type = other DNA
aatgtacagtattgcgttttggagaaagatcaggaacacttgtcccctactag

SEQ ID NO: 217 - DNA, 48 bp, synthetic construct
  misc_feature 1..48: note = Primer
  source 1..48: mol_type = other DNA
aatgtacagtattgcgttttggtcctccacgatctcctcatactcctc

SEQ ID NO: 218 - DNA, 47 bp, synthetic construct
  misc_feature 1..47: note = Primer
  source 1..47: mol_type = other DNA
aatgtacagtattgcgttttgtcgatggacttgacaagcccgtactt

SEQ ID NO: 219 - DNA, 48 bp, synthetic construct
  misc_feature 1..48: note = Primer
  source 1..48: mol_type = other DNA
aatgtacagtattgcgttttgctggacgacgaggagtatgaggagatc

SEQ ID NO: 220 - DNA, 46 bp, synthetic construct
  misc_feature 1..46: note = Primer
  source 1..46: mol_type = other DNA
aatgtacagtattgcgttttgtaccagaagtcccggcggtgataag

SEQ ID NO: 221 - DNA, 49 bp, synthetic construct
  misc_feature 1..49: note = Primer
  source 1..49: mol_type = other DNA
aatgtacagtattgcgttttggttcacctctgtgtttgactgccagaaa

SEQ ID NO: 222 - DNA, 57 bp, synthetic construct
  misc_feature 1..57: note = Primer
  source 1..57: mol_type = other DNA
aatgtacagtattgcgttttgcaatgagtattctcttcatttcaggtcagttgattt

SEQ ID NO: 223 - DNA, 49 bp, synthetic construct
  misc_feature 1..49: note = Primer
  source 1..49: mol_type = other DNA
aatgtacagtattgcgttttgggctgctttcttgaaggctattgggtat

SEQ ID NO: 224 - DNA, 53 bp, synthetic construct
  misc_feature 1..53: note = Primer
  source 1..53: mol_type = other DNA
aatgtacagtattgcgttttgaggagactggaattctcgaataaggattaaca

SEQ ID NO: 225 - DNA, 57 bp, synthetic construct
  misc_feature 1..57: note = Primer
  source 1..57: mol_type = other DNA
aatgtacagtattgcgttttggcatagttaaaacctgtgtttggttttgtaggtctt

SEQ ID NO: 226 - DNA, 48 bp, synthetic construct
  misc_feature 1..48: note = Primer
  source 1..48: mol_type = other DNA
aatgtacagtattgcgttttgctctgtgttggcggatacccttccata

SEQ ID NO: 227 - DNA, 53 bp, synthetic construct
  misc_feature 1..53: note = Primer
  source 1..53: mol_type = other DNA
aatgtacagtattgcgttttgggcattccttctttattgcccttcttaaaagc

SEQ ID NO: 228 - DNA, 50 bp, synthetic construct
  misc_feature 1..50: note = Primer
  source 1..50: mol_type = other DNA
aatgtacagtattgcgttttggctgctggtctggctactatgatctctac

SEQ ID NO: 229 - DNA, 53 bp, synthetic construct
  misc_feature 1..53: note = Primer
  source 1..53: mol_type = other DNA
aatgtacagtattgcgttttggcacacagcttttaagaagggcaataaagaag

SEQ ID NO: 230 - DNA, 57 bp, synthetic construct
  misc_feature 1..57: note = Primer
  source 1..57: mol_type = other DNA
aatgtacagtattgcgttttgtgtatgtttaattctgtacatgagcatttcatcagt

SEQ ID NO: 231 - DNA, 57 bp, synthetic construct
  misc_feature 1..57: note = Primer
  source 1..57: mol_type = other DNA
aatgtacagtattgcgttttgatttcataccttgcttaatgggtgtagataccaaaa

SEQ ID NO: 232 - DNA, 48 bp, synthetic construct
  misc_feature 1..48: note = Primer
  source 1..48: mol_type = other DNA
aatgtacagtattgcgttttgttggcgtcaaatgtgccactatcactc

SEQ ID NO: 233 - DNA, 57 bp, synthetic construct
  misc_feature 1..57: note = Primer
  source 1..57: mol_type = other DNA
aatgtacagtattgcgttttgttctctttcaagctatgatttaggcatagagaatcg

SEQ ID NO: 234 - DNA, 57 bp, synthetic construct
  misc_feature 1..57: note = Primer
  source 1..57: mol_type = other DNA
aatgtacagtattgcgttttgctgcagttgtaggttataactatccatttgtctgaa

SEQ ID NO: 235 - DNA, 52 bp, synthetic construct
  misc_feature 1..52: note = Primer
  source 1..52: mol_type = other DNA
aatgtacagtattgcgttttgccctaggtcagatcacccagtcagttaaaac

SEQ ID NO: 236 - DNA, 52 bp, synthetic construct
  misc_feature 1..52: note = Primer
  source 1..52: mol_type = other DNA
aatgtacagtattgcgttttgtggttaaaggtcagcccacttaccagatatg

SEQ ID NO: 237 - DNA, 49 bp, synthetic construct
  misc_feature 1..49: note = Primer
  source 1..49: mol_type = other DNA
aatgtacagtattgcgttttggggtatgctccccatttagaggataagg

SEQ ID NO: 238 - DNA, 50 bp, synthetic construct
  misc_feature 1..50: note = Primer
  source 1..50: mol_type = other DNA
aatgtacagtattgcgttttgacgtcagatctacagcgaacacaactact

SEQ ID NO: 239 - DNA, 50 bp, synthetic construct
  misc_feature 1..50: note = Primer
  source 1..50: mol_type = other DNA
aatgtacagtattgcgttttgagtggtgccagactcacattcagttctaa

SEQ ID NO: 240 - DNA, 54 bp, synthetic construct
  misc_feature 1..54: note = Primer
  source 1..54: mol_type = other DNA
aatgtacagtattgcgttttgcttggccagttcctttctctaatgtatcatctc

SEQ ID NO: 241 - DNA, 56 bp, synthetic construct
  misc_feature 1..56: note = Primer
  source 1..56: mol_type = other DNA
aatgtacagtattgcgttttgaagttttcttgtctagtatcactttccctcatagg

SEQ ID NO: 242 - DNA, 49 bp, synthetic construct
  misc_feature 1..49: note = Primer
  source 1..49: mol_type = other DNA
aatgtacagtattgcgttttggggctcaacagatggtatgtgttctctg

SEQ ID NO: 243 - DNA, 54 bp, synthetic construct
  misc_feature 1..54: note = Primer
  source 1..54: mol_type = other DNA
aatgtacagtattgcgttttggctctcgtttctaacagttctttgcattggata

SEQ ID NO: 244 - DNA, 50 bp, synthetic construct
  misc_feature 1..50: note = Primer
  source 1..50: mol_type = other DNA
aatgtacagtattgcgttttggaggtgaccttcaaagtcagaggctgtat

SEQ ID NO: 245 - DNA, 49 bp, synthetic construct
  misc_feature 1..49: note = Primer
  source 1..49: mol_type = other DNA
aatgtacagtattgcgttttggagcaaccatcccatctgtccttgtaac

SEQ ID NO: 246 - DNA, 50 bp, synthetic construct
  misc_feature 1..50: note = Primer
  source 1..50: mol_type = other DNA
aatgtacagtattgcgttttgggacaaggatgagaaacccaattggaacc

SEQ ID NO: 247 - DNA, 46 bp, synthetic construct
  misc_feature 1..46: note = Primer
  source 1..46: mol_type = other DNA
aatgtacagtattgcgttttgcggtccgccaaaagatcccagattc

SEQ ID NO: 248 - DNA, 46 bp, synthetic construct
  misc_feature 1..46: note = Primer
  source 1..46: mol_type = other DNA
aatgtacagtattgcgttttgggaggccactaacccacttgtgatg

SEQ ID NO: 249 - DNA, 54 bp, synthetic construct
  misc_feature 1..54: note = Primer
  source 1..54: mol_type = other DNA
aatgtacagtattgcgttttgtccagtttcctagaggatgtaatgggatttgtc

SEQ ID NO: 250 - DNA, 52 bp, synthetic construct
  misc_feature 1..52: note = Primer
  source 1..52: mol_type = other DNA
aatgtacagtattgcgttttgtcacatttggagatgagaaacgaggtgttct

SEQ ID NO: 251 - DNA, 48 bp, synthetic construct
  misc_feature 1..48: note = Primer
  source 1..48: mol_type = other DNA
aatgtacagtattgcgttttgcccttggcctgtaacattgctctgatc

SEQ ID NO: 252 - DNA, 52 bp, synthetic construct
  misc_feature 1..52: note = Primer
  source 1..52: mol_type = other DNA
aatgtacagtattgcgttttgcacctcgtttctcatctccaaatgtgatctc

SEQ ID NO: 253 - DNA, 47 bp, synthetic construct
  misc_feature 1..47: note = Primer
  source 1..47: mol_type = other DNA
aatgtacagtattgcgttttgccagtagctttcctgttctcggcatt

SEQ ID NO: 254 - DNA, 50 bp, synthetic construct
  misc_feature 1..50: note = Primer
  source 1..50: mol_type = other DNA
aatgtacagtattgcgttttggcagcgtcaagaatgagaagacttttgtg

SEQ ID NO: 255 - DNA, 47 bp, synthetic construct
  misc_feature 1..47: note = Primer
  source 1..47: mol_type = other DNA
aatgtacagtattgcgttttgttgcccttctggaaattaccccgaga

SEQ ID NO: 256 - DNA, 55 bp, synthetic construct
  misc_feature 1..55: note = Primer
  source 1..55: mol_type = other DNA
aatgtacagtattgcgttttgagttccaccagctttaattattcctctagctctc

SEQ ID NO: 257 - DNA, 56 bp, synthetic construct
  misc_feature 1..56: note = Primer
  source 1..56: mol_type = other DNA
aatgtacagtattgcgttttggtttcccatggccataatttattatctcaccacaa

SEQ ID NO: 258 - DNA, 48 bp, synthetic construct
  misc_feature 1..48: note = Primer
  source 1..48: mol_type = other DNA
aatgtacagtattgcgttttggtcacgatgactgtattggaccctcaa

SEQ ID NO: 259 - DNA, 56 bp, synthetic construct
  misc_feature 1..56: note = Primer
  source 1..56: mol_type = other DNA
aatgtacagtattgcgttttgtccagacctttgctttagattggcaattattactg

SEQ ID NO: 260 - DNA, 51 bp, synthetic construct
  misc_feature 1..51: note = Primer
  source 1..51: mol_type = other DNA
aatgtacagtattgcgttttgccctaacaacacagaagcaaagcgttcttt

SEQ ID NO: 261 - DNA, 45 bp, synthetic construct
  misc_feature 1..45: note = Primer
  source 1..45: mol_type = other DNA
aatgtacagtattgcgttttgcgccctcctaccacctgtactacg

SEQ ID NO: 262 - DNA, 46 bp, synthetic construct
  misc_feature 1..46: note = Primer
  source 1..46: mol_type = other DNA
aatgtacagtattgcgttttgactatccaggcgccttcacctactc

SEQ ID NO: 263 - DNA, 47 bp, synthetic construct
  misc_feature 1..47: note = Primer
  source 1..47: mol_type = other DNA
aatgtacagtattgcgttttgctcctaggcggtatcatcctgggtag

SEQ ID NO: 264 - DNA, 51 bp, synthetic construct
  misc_feature 1..51: note = Primer
  source 1..51: mol_type = other DNA
aatgtacagtattgcgttttgtctgattctcttcagatacaaggcagatcc

SEQ ID NO: 265 - DNA, 55 bp, synthetic construct
  misc_feature 1..55: note = Primer
  source 1..55: mol_type = other DNA
aatgtacagtattgcgttttggcagatacttggacttgagtaggcttattaaacc

SEQ ID NO: 266 - DNA, 57 bp, synthetic construct
  misc_feature 1..57: note = Primer
  source 1..57: mol_type = other DNA
aatgtacagtattgcgttttggcggctctataaagaattgtccttattttcgaactt

SEQ ID NO: 267 - DNA, 48 bp, synthetic construct
  misc_feature 1..48: note = Primer
  source 1..48: mol_type = other DNA
aatgtacagtattgcgttttggttcgaggcctttctctgagcatcaag

SEQ ID NO: 268 - DNA, 51 bp, synthetic construct
  misc_feature 1..51: note = Primer
  source 1..51: mol_type = other DNA
aatgtacagtattgcgttttgacatcggcagaaactagatgatcagaccaa

SEQ ID NO: 269 - DNA, 57 bp, synthetic construct
  misc_feature 1..57: note = Primer
  source 1..57: mol_type = other DNA
aatgtacagtattgcgttttgtttaggaaatccacaatactttttctgatctcttcc

SEQ ID NO: 270 - DNA, 53 bp, synthetic construct
  misc_feature 1..53: note = Primer
  source 1..53: mol_type = other DNA
aatgtacagtattgcgttttggccaccaacctcattctgttttgttctctatc

SEQ ID NO: 271 - DNA, 51 bp, synthetic construct
  misc_feature 1..51: note = Primer
  source 1..51: mol_type = other DNA
aatgtacagtattgcgttttgctgcatttgtcctttgactggtgtttaggt

SEQ ID NO: 272 - DNA, 52 bp, synthetic construct
  misc_feature 1..52: note = Primer
  source 1..52: mol_type = other DNA
aatgtacagtattgcgttttgcttcgaccgacaaacctgaggtcattaaatc

SEQ ID NO: 273 - DNA, 46 bp, synthetic construct
  misc_feature 1..46: note = Primer
  source 1..46: mol_type = other DNA
aatgtacagtattgcgttttgccccacatcccaagctaggaagacc

SEQ ID NO: 274 - DNA, 45 bp, synthetic construct
  misc_feature 1..45: note = Primer
  source 1..45: mol_type = other DNA
aatgtacagtattgcgttttgcgggccagtaccttgaaagcgatg

SEQ ID NO: 275 - DNA, 51 bp, synthetic construct
  misc_feature 1..51: note = Primer
  source 1..51: mol_type = other DNA
aatgtacagtattgcgttttgctaactcaatcggcttgttgtgatgcgtat

SEQ ID NO: 276 - DNA, 51 bp, synthetic construct
  misc_feature 1..51: note = Primer
  source 1..51: mol_type = other DNA
aatgtacagtattgcgttttgccctcctggactgttagtaacttagtctcc

SEQ ID NO: 277 - DNA, 42 bp, synthetic construct
  misc_feature 1..42: note = Primer
  source 1..42: mol_type = other DNA
aatgtacagtattgcgttttgccctccgagctccgcgaaaat

SEQ ID NO: 278 - DNA, 57 bp, synthetic construct
  misc_feature 1..57: note = Primer
  source 1..57: mol_type = other DNA
aatgtacagtattgcgttttggtgctaaaaagtgtaagaagaaatgagctagcaaaa

SEQ ID NO: 279 - DNA, 55 bp, synthetic construct
  misc_feature 1..55: note = Primer
  source 1..55: mol_type = other DNA
aatgtacagtattgcgttttgcatatgcctcagtttgaattcctctcacaaacaa

SEQ ID NO: 280 - DNA, 51 bp, synthetic construct
  misc_feature 1..51: note = Primer
  source 1..51: mol_type = other DNA
aatgtacagtattgcgttttggggagaagaaagagagatgtagggctagag

SEQ ID NO: 281 - DNA, 54 bp, synthetic construct
  misc_feature 1..54: note = Primer
  source 1..54: mol_type = other DNA
aatgtacagtattgcgttttggcaagcacttctgtttttgtcttttcagtttcg

SEQ ID NO: 282 - DNA, 57 bp, synthetic construct
  misc_feature 1..57: note = Primer
  source 1..57: mol_type = other DNA
aatgtacagtattgcgttttgtctctgatatacttggattggtaattgagaaagtct

SEQ ID NO: 283 - DNA, 57 bp, synthetic construct
  misc_feature 1..57: note = Primer
  source 1..57: mol_type = other DNA
aatgtacagtattgcgttttggtttgatatcttcccagcaaaataatcagctctcat

SEQ ID NO: 284 - DNA, 51 bp, synthetic construct
  misc_feature 1..51: note = Primer
  source 1..51: mol_type = other DNA
aatgtacagtattgcgttttgtagccaacctcttttcgatgagctcactag

SEQ ID NO: 285 - DNA, 51 bp, synthetic construct
  misc_feature 1..51: note = Primer
  source 1..51: mol_type = other DNA
aatgtacagtattgcgttttgtggaacagacaaactatcgactgaagttgt

SEQ ID NO: 286 - DNA, 48 bp, synthetic construct
  misc_feature 1..48: note = Primer
  source 1..48: mol_type = other DNA
aatgtacagtattgcgttttggaggctgagtgcaaatttggtctggaa

SEQ ID NO: 287 - DNA, 50 bp, synthetic construct
  misc_feature 1..50: note = Primer
  source 1..50: mol_type = other DNA
aatgtacagtattgcgttttggatggtggtggttgtctctgatgattacc

SEQ ID NO: 288 - DNA, 46 bp, synthetic construct
  misc_feature 1..46: note = Primer
  source 1..46: mol_type = other DNA
aatgtacagtattgcgttttggcaaggcgagtccagaaccaagatt

SEQ ID NO: 289 - DNA, 47 bp, synthetic construct
  misc_feature 1..47: note = Primer
  source 1..47: mol_type = other DNA
aatgtacagtattgcgttttgtcagaagcgactgatccccatcaagt

SEQ ID NO: 290 - DNA, 56 bp, synthetic construct
  misc_feature 1..56: note = Primer
  source 1..56: mol_type = other DNA
aatgtacagtattgcgttttgcatatggtcacatcaccttaactaaacccatgttt

SEQ ID NO: 291 - DNA, 57 bp, synthetic construct
  misc_feature 1..57: note = Primer
  source 1..57: mol_type = other DNA
aatgtacagtattgcgttttgtttctcggtactgtttattttgaacaaaaccaatcc

SEQ ID NO: 292 - DNA, 51 bp, synthetic construct
  misc_feature 1..51: note = Primer
  source 1..51: mol_type = other DNA
aatgtacagtattgcgttttgcctcctccccaaattccaggaacaatatga

SEQ ID NO: 293 - DNA, 57 bp, synthetic construct
  misc_feature 1..57: note = Primer
  source 1..57: mol_type = other DNA
aatgtacagtattgcgttttgtgtgcgtcattttatttgggaaaatttgatactaac

SEQ ID NO: 294 - DNA, 46 bp, synthetic construct
  misc_feature 1..46: note = Primer
  source 1..46: mol_type = other DNA
aatgtacagtattgcgttttgcatgcaggagaagtcatcccccttc

SEQ ID NO: 295 - DNA, 51 bp, synthetic construct
  misc_feature 1..51: note = Primer
  source 1..51: mol_type = other DNA
aatgtacagtattgcgttttgtctgaaaactggtggttgcctctaggttaa

SEQ ID NO: 296 - DNA, 48 bp, synthetic construct
  misc_feature 1..48: note = Primer
  source 1..48: mol_type = other DNA
aatgtacagtattgcgttttggcccctttcttgctcttcttggacttg

SEQ ID NO: 297 - DNA, 48 bp, synthetic construct
  misc_feature 1..48: note = Primer
  source 1..48: mol_type = other DNA
aatgtacagtattgcgttttgccaagccaagccaagctggatattgtg

SEQ ID NO: 298 - DNA, 49 bp, synthetic construct
  misc_feature 1..49: note = Primer
  source 1..49: mol_type = other DNA
aatgtacagtattgcgttttgcactcacattgtgcagcttgtagtagag

SEQ ID NO: 299 - DNA, 48 bp, synthetic construct
  misc_feature 1..48: note = Primer
  source 1..48: mol_type = other DNA
aatgtacagtattgcgttttggcaaagcgtctgcatttgaaggagttt

SEQ ID NO: 300 - DNA, 45 bp, synthetic construct
  misc_feature 1..45: note = Primer
  source 1..45: mol_type = other DNA
aatgtacagtattgcgttttgccctcccgagaacttgccggttaa

SEQ ID NO: 301 - DNA, 46 bp, synthetic construct
  misc_feature 1..46: note = Primer
  source 1..46: mol_type = other DNA
aatgtacagtattgcgttttggctccccaccacaaaaacgcaaatg

SEQ ID NO: 302 - DNA, 48 bp, synthetic construct
  misc_feature 1..48: note = Primer
  source 1..48: mol_type = other DNA
aatgtacagtattgcgttttggtgtcactgacggagagcatgaagatg

SEQ ID NO: 303 - DNA, 46 bp, synthetic construct
  misc_feature 1..46: note = Primer
  source 1..46: mol_type = other DNA
aatgtacagtattgcgttttgccacccaaagaagtgtctcctgacc

SEQ ID NO: 304 - DNA, 47 bp, synthetic construct
  misc_feature 1..47: note = Primer
  source 1..47: mol_type = other DNA
aatgtacagtattgcgttttgtccgtcagtgacacctggtacttgac

SEQ ID NO: 305 - DNA, 48 bp, synthetic construct
  misc_feature 1..48: note = Primer
  source 1..48: mol_type = other DNA
aatgtacagtattgcgttttgccctagctctgcctaccctgatctttc

SEQ ID NO: 306 - DNA, 47 bp, synthetic construct
  misc_feature 1..47: note = Primer
  source 1..47: mol_type = other DNA
aatgtacagtattgcgttttgacgaggtggacgtcttcttcaatcac

SEQ ID NO: 307 - DNA, 43 bp, synthetic construct
  misc_feature 1..43: note = Primer
  source 1..43: mol_type = other DNA
aatgtacagtattgcgttttggccctgcgagtcgaggtgattg

SEQ ID NO: 308 - DNA, 53 bp, synthetic construct
  misc_feature 1..53: note = Primer
  source 1..53: mol_type = other DNA
aatgtacagtattgcgttttgccatgactctcaggaattggccctatacttag

SEQ ID NO: 309 - DNA, 56 bp, synthetic construct
  misc_feature 1..56: note = Primer
  source 1..56: mol_type = other DNA
aatgtacagtattgcgttttgcttgggaccttcatttctatataacccctatctgg

SEQ ID NO: 310 - DNA, 49 bp, synthetic construct
  misc_feature 1..49: note = Primer
  source 1..49: mol_type = other DNA
aatgtacagtattgcgttttgtgccaggaaacttttcattgtgcctctc

SEQ ID NO: 311 - DNA, 50 bp, synthetic construct
  misc_feature 1..50: note = Primer
  source 1..50: mol_type = other DNA
aatgtacagtattgcgttttggttaccccatggaacttaccaagcactag

SEQ ID NO: 312 - DNA, 54 bp, synthetic construct
  misc_feature 1..54: note = Primer
  source 1..54: mol_type = other DNA
aatgtacagtattgcgttttggtatgaaattcgctggagggtcattgaatcaat

SEQ ID NO: 313 - DNA, 52 bp, synthetic construct
  misc_feature 1..52: note = Primer
  source 1..52: mol_type = other DNA
aatgtacagtattgcgttttgcaggaaggagcacttacgttttagcatcttc

SEQ ID NO: 314 - DNA, 57 bp, synthetic construct
  misc_feature 1..57: note = Primer
  source 1..57: mol_type = other DNA
aatgtacagtattgcgttttggattttgagaaattcccttaatatccccatgctcaa

SEQ ID NO: 315 - DNA, 49 bp, synthetic construct
  misc_feature 1..49: note = Primer
  source 1..49: mol_type = other DNA
aatgtacagtattgcgttttgcacaaccacatgtgtccagtgaaaatcc

SEQ ID NO: 316 - DNA, 49 bp, synthetic construct
  misc_feature 1..49: note = Primer
  source 1..49: mol_type = other DNA
aatgtacagtattgcgttttgtgctttcatcagcagggttcaatccaaa

SEQ ID NO: 317 - DNA, 57 bp, synthetic construct
  misc_feature 1..57: note = Primer
  source 1..57: mol_type = other DNA
aatgtacagtattgcgttttgcatttacatcatcacagagtattgcttctatggaga

SEQ ID NO: 318 - DNA, 56 bp, synthetic construct
  misc_feature 1..56: note = Primer
  source 1..56: mol_type = other DNA
aatgtacagtattgcgttttggtgatctctggatgtcggaatatttagaaacctct

SEQ ID NO: 319 - DNA, 57 bp, synthetic construct
  misc_feature 1..57: note = Primer
  source 1..57: mol_type = other DNA
aatgtacagtattgcgttttgatcttttgaaaacaatggtgactacatggacatgaa

SEQ ID NO: 320 - DNA, 54 bp, synthetic construct
  misc_feature 1..54: note = Primer
  source 1..54: mol_type = other DNA
aatgtacagtattgcgttttgggtctaaaaaggtctgtgttccttgaacttaca

SEQ ID NO: 321 - DNA, 56 bp, synthetic construct
  misc_feature 1..56: note = Primer
  source 1..56: mol_type = other DNA
aatgtacagtattgcgttttgccagcaccaatacatttaatttcttttctgcagac

SEQ ID NO: 322 - DNA, 51 bp, synthetic construct
  misc_feature 1..51: note = Primer
  source 1..51: mol_type = other DNA
aatgtacagtattgcgttttggctacagatggcttgatcctgagtcatttc

SEQ ID NO: 323 - DNA, 48 bp, synthetic construct
  misc_feature 1..48: note = Primer
  source 1..48: mol_type = other DNA
aatgtacagtattgcgttttggtcaggcccataccaagggaaaagatc

SEQ ID NO: 324 - DNA, 51 bp, synthetic construct
  misc_feature 1..51: note = Primer
  source 1..51: mol_type = other DNA
aatgtacagtattgcgttttgacactgagtgatgtctggtcttatggcatt

SEQ ID NO: 325 - DNA, 50 bp, synthetic construct
  misc_feature 1..50: note = Primer
  source 1..50: mol_type = other DNA
aatgtacagtattgcgttttgcactgagcgtttgttagtcctggtgtttt

SEQ ID NO: 326 - DNA, 53 bp, synthetic construct
  misc_feature 1..53: note = Primer
  source 1..53: mol_type = other DNA
aatgtacagtattgcgttttgcagattctccacaatctcactcaggtggtaaa

SEQ ID NO: 327 - DNA, 49 bp, synthetic construct
  misc_feature 1..49: note = Primer
  source 1..49: mol_type = other DNA
aatgtacagtattgcgttttgccccacagctacgagatcatggtgaaat

SEQ ID NO: 328 - DNA, 57 bp, synthetic construct
  misc_feature 1..57: note = Primer
  source 1..57: mol_type = other DNA
aatgtacagtattgcgttttgtctctattcatttttgaggtttggttgttaacactt

SEQ ID NO: 329 - DNA, 48 bp, synthetic construct
  misc_feature 1..48: note = Primer
  source 1..48: mol_type = other DNA
aatgtacagtattgcgttttggggagtgcaccattatcgggaaaatgg

SEQ ID NO: 330 - DNA, 56 bp, synthetic construct
  misc_feature 1..56: note = Primer
  source 1..56: mol_type = other DNA
aatgtacagtattgcgttttggcttattctcattcgtttcatccaggatctcaaaa

SEQ ID NO: 331 - DNA, 46 bp, synthetic construct
  misc_feature 1..46: note = Primer
  source 1..46: mol_type = other DNA
aatgtacagtattgcgttttggggcgacgagattaggctgttatgc

SEQ ID NO: 332 - DNA, 49 bp, synthetic construct
  misc_feature 1..49: note = Primer
  source 1..49: mol_type = other DNA
aatgtacagtattgcgttttgcccctctgcattataagcagtgccaaaa

SEQ ID NO: 333 - DNA, 50 bp, synthetic construct
  misc_feature 1..50: note = Primer
  source 1..50: mol_type = other DNA
aatgtacagtattgcgttttggcccacatcgttgtaagccttacattcaa

SEQ ID NO: 334 - DNA, 50 bp, synthetic construct
  misc_feature 1..50: note = Primer
  source 1..50: mol_type = other DNA
aatgtacagtattgcgttttgccgtttggaaagctagtggttcagagttc

SEQ ID NO: 335 - DNA, 50 bp, synthetic construct
  misc_feature 1..50: note = Primer
  source 1..50: mol_type = other DNA
aatgtacagtattgcgttttggagatcccatcctgccaaagtttgtgatt

SEQ ID NO: 336 - DNA, 50 bp, synthetic construct
  misc_feature 1..50: note = Primer
  source 1..50: mol_type = other DNA
aatgtacagtattgcgttttgggaaagcccctgtttcatactgaccaaaa

SEQ ID NO: 337 - DNA, 51 bp, synthetic construct
  misc_feature 1..51: note = Primer
  source 1..51: mol_type = other DNA
aatgtacagtattgcgttttgctttctccccacagaaacccatgtatgaag

SEQ ID NO: 338 - DNA, 51 bp, synthetic construct
  misc_feature 1..51: note = Primer
  source 1..51: mol_type = other DNA
aatgtacagtattgcgttttggtttgccagttgtgctttttgctaaaatgc

SEQ ID NO: 339 - DNA, 48 bp, synthetic construct
  misc_feature 1..48: note = Primer
  source 1..48: mol_type = other DNA
aatgtacagtattgcgttttgccctcccaccctcaggactataccaat

SEQ ID NO: 340 - DNA, 46 bp, synthetic construct
  misc_feature 1..46: note = Primer
  source 1..46: mol_type = other DNA
aatgtacagtattgcgttttgtgctcggcagattggtatagtcctg

SEQ ID NO: 341 - DNA, 50 bp, synthetic construct
  misc_feature 1..50: note = Primer
  source 1..50: mol_type = other DNA
aatgtacagtattgcgttttgggcatcctctgtcctatctcccagataca

SEQ ID NO: 342 - DNA, 56 bp, synthetic construct
  misc_feature 1..56: note = Primer
  source 1..56: mol_type = other DNA
aatgtacagtattgcgttttgaggttttatactaaacttactttgactgggtttgg

SEQ ID NO: 343 - DNA, 45 bp, synthetic construct
  misc_feature 1..45: note = Primer
  source 1..45: mol_type = other DNA
aatgtacagtattgcgttttgcccccagaggtaagcgtcatatgg

SEQ ID NO: 344 - DNA, 49 bp, synthetic construct
  misc_feature 1..49: note = Primer
  source 1..49: mol_type = other DNA
aatgtacagtattgcgttttggcacagggaagtaggtactgggagattg

SEQ ID NO: 345 - DNA, 48 bp, synthetic construct
  misc_feature 1..48: note = Primer
  source 1..48: mol_type = other DNA
aatgtacagtattgcgttttgaggcctgcaaggttttaactggaccta

SEQ ID NO: 346 - DNA, 49 bp, synthetic construct
  misc_feature 1..49: note = Primer
  source 1..49: mol_type = other DNA
aatgtacagtattgcgttttgcgggagctgataagtggtacctgtatgt

SEQ ID NO: 347 - DNA, 49 bp, synthetic construct
  misc_feature 1..49: note = Primer
  source 1..49: mol_type = other DNA
aatgtacagtattgcgttttggaaaagggtcccaggtaggtccagttaa

SEQ ID NO: 348 - DNA, 57 bp, synthetic construct
  misc_feature 1..57: note = Primer
  source 1..57: mol_type = other DNA
aatgtacagtattgcgttttgctctcggtgtatttctctacttacctgtaataatgc

SEQ ID NO: 349 - DNA, 57 bp, synthetic construct
  misc_feature 1..57: note = Primer
  source 1..57: mol_type = other DNA
aatgtacagtattgcgttttgtttattgatgtctatgaagtgttgtggttccttaac

SEQ ID NO: 350 - DNA, 49 bp, synthetic construct
  misc_feature 1..49: note = Primer
  source 1..49: mol_type = other DNA
aatgtacagtattgcgttttgcagaaaacaagctgccgcaaagttctac

SEQ ID NO: 351 - DNA, 48 bp, synthetic construct
  misc_feature 1..48: note = Primer
  source 1..48: mol_type = other DNA
aatgtacagtattgcgttttgcaggtgttgcgatgatgtcactgtacg

SEQ ID NO: 352 - DNA, 57 bp, synthetic construct
  misc_feature 1..57: note = Primer
  source 1..57: mol_type = other DNA
aatgtacagtattgcgttttgtcatttttcattggacttgttttgtcagctttttgg

SEQ ID NO: 353 - DNA, 55 bp, synthetic construct
  misc_feature 1..55: note = Primer
  source 1..55: mol_type = other DNA
aatgtacagtattgcgttttggttagccccaatatgaaaaataaagctggttgga

SEQ ID NO: 354 - DNA, 53 bp, synthetic construct
  misc_feature 1..53: note = Primer
  source 1..53: mol_type = other DNA
aatgtacagtattgcgttttgctggttggaggtttttgctaaatctggaatga

SEQ ID NO: 355 - DNA, 57 bp, synthetic construct
  misc_feature 1..57: note = Primer
  source 1..57: mol_type = other DNA
aatgtacagtattgcgttttgttctttttgactagaaaacttcagccactgtgtatt

SEQ ID NO: 356 - DNA, 55 bp, synthetic construct
  misc_feature 1..55: note = Primer
  source 1..55: mol_type = other DNA
aatgtacagtattgcgttttgcatatgaccaattgcagatgagcccattattgaa

SEQ ID NO: 357 - DNA, 51 bp, synthetic construct
  misc_feature 1..51: note = Primer
  source 1..51: mol_type = other DNA
aatgtacagtattgcgttttgaggcatagctgactcatctatgtttgttct

SEQ ID NO: 358 - DNA, 57 bp, synthetic construct
  misc_feature 1..57: note = Primer
  source 1..57: mol_type = other DNA
aatgtacagtattgcgttttgttcctcatttctttcactctgacagtataaaggtaa

SEQ ID NO: 359 - DNA, 54 bp, synthetic construct
  misc_feature 1..54: note = Primer
  source 1..54: mol_type = other DNA
aatgtacagtattgcgttttggaactattccaacagaacaaaccgataacatca

SEQ ID NO: 360 - DNA, 53 bp, synthetic construct
  misc_feature 1..53: note = Primer
  source 1..53: mol_type = other DNA
aatgtacagtattgcgttttgtggatagcaagacaattagagcccaacttagt

SEQ ID NO: 361 - DNA, 54 bp, synthetic construct
  misc_feature 1..54: note = Primer
  source 1..54: mol_type = other DNA
aatgtacagtattgcgttttgctactcctcctgtctctttccacatcatcaatt

SEQ ID NO: 362 - DNA, 57 bp, synthetic construct
  misc_feature 1..57: note = Primer
  source 1..57: mol_type = other DNA
aatgtacagtattgcgttttgaggaccttatgttgtatgctgtataaatctaaaggt

SEQ ID NO: 363 - DNA, 57 bp, synthetic construct
  misc_feature 1..57: note = Primer
  source 1..57: mol_type = other DNA
aatgtacagtattgcgttttggtttgtcatcttctatggtaagtatctttctggatg

SEQ ID NO: 364 - DNA, 57 bp, synthetic construct
  misc_feature 1..57: note = Primer
  source 1..57: mol_type = other DNA
aatgtacagtattgcgttttgtggaggagaaacagataaaagttgagtatacgttta

SEQ ID NO: 365 - DNA, 54 bp, synthetic construct
  misc_feature 1..54: note = Primer
  source 1..54: mol_type = other DNA
aatgtacagtattgcgttttggaggatgacgacatgttagtaagcactactact

SEQ ID NO: 366 - DNA, 57 bp, synthetic construct
  misc_feature 1..57: note = Primer
  source 1..57: mol_type = other DNA
aatgtacagtattgcgttttgattccaccatcatttccttctccaaaattatcatcc

SEQ ID NO: 367 - DNA, 53 bp, synthetic construct
  misc_feature 1..53: note = Primer
  source 1..53: mol_type = other DNA
aatgtacagtattgcgttttgctcaaaagcactgccttctctcattatctcac

SEQ ID NO: 368 - DNA, 57 bp, synthetic construct
  misc_feature 1..57: note = Primer
  source 1..57: mol_type = other DNA
aatgtacagtattgcgttttgaatgtatttgaccttcttttaaagtgacatcgatgt

SEQ ID NO: 369 - DNA, 55 bp, synthetic construct
  misc_feature 1..55: note = Primer
  source 1..55: mol_type = other DNA
aatgtacagtattgcgttttgtgatgttcccaacttcttctctcatggttatctc

SEQ ID NO: 370 - DNA, 56 bp, synthetic construct
  misc_feature 1..56: note = Primer
  source 1..56: mol_type = other DNA
aatgtacagtattgcgttttgccctctgatccctagataatttatgggtagctaga

SEQ ID NO: 371 - DNA, 55 bp, synthetic construct
  misc_feature 1..55: note = Primer
  source 1..55: mol_type = other DNA
aatgtacagtattgcgttttgcacgaaatgcaggttttggaatatgattaatgtt

SEQ ID NO: 372 - DNA, 56 bp, synthetic construct
  misc_feature 1..56: note = Primer
  source 1..56: mol_type = other DNA
aatgtacagtattgcgttttggaacaatgttctacgcacattttgttctcagtaaa

SEQ ID NO: 373 - DNA, 50 bp, synthetic construct
  misc_feature 1..50: note = Primer
  source 1..50: mol_type = other DNA
aatgtacagtattgcgttttgtccacgctgctctctaaattacactcgaa

SEQ ID NO: 374 - DNA, 54 bp, synthetic construct
  misc_feature 1..54: note = Primer
  source 1..54: mol_type = other DNA
aatgtacagtattgcgttttgacgtagaacacatttcattttactcctctttgg

SEQ ID NO: 375 - DNA, 57 bp, synthetic construct
  misc_feature 1..57: note = Primer
  source 1..57: mol_type = other DNA
aatgtacagtattgcgttttggtcacatgaatgtaaatcaagaaaacagatgttgtt

SEQ ID NO: 376 - DNA, 57 bp, synthetic construct
  misc_feature 1..57: note = Primer
  source 1..57: mol_type = other DNA
aatgtacagtattgcgttttgttctgaactatttatggacaacagtcaaacaacaat

SEQ ID NO: 377 - DNA, 57 bp, synthetic construct
  misc_feature 1..57: note = Primer
  source 1..57: mol_type = other DNA
aatgtacagtattgcgttttgtgaagccattgcgagaactttatccataagtatttc

SEQ ID NO: 378 - DNA, 51 bp, synthetic construct
  misc_feature 1..51: note = Primer
  source 1..51: mol_type = other DNA
aatgtacagtattgcgttttggccagagcacatgaataaatgagcatccat

SEQ ID NO: 379 - DNA, 53 bp, synthetic construct
  misc_feature 1..53: note = Primer
  source 1..53: mol_type = other DNA
aatgtacagtattgcgttttgggaagctctcagggtacaaattctcagatcat

SEQ ID NO: 380 - DNA, 54 bp, synthetic construct
  misc_feature 1..54: note = Primer
  source 1..54: mol_type = other DNA
aatgtacagtattgcgttttgctcagggtacaaattctcagatcatcagtcctc

SEQ ID NO: 381 - DNA, 49 bp, synthetic construct
  misc_feature 1..49: note = Primer
  source 1..49: mol_type = other DNA
aatgtacagtattgcgttttgctctacacaagcttcctttccgtcatgc

SEQ ID NO: 382 - DNA, 52 bp, synthetic construct
  misc_feature 1..52: note = Primer
  source 1..52: mol_type = other DNA
aatgtacagtattgcgttttgcccttcagatcttctcagcattcgagagatc

SEQ ID NO: 383 - DNA, 49 bp, synthetic construct
  misc_feature 1..49: note = Primer
  source 1..49: mol_type = other DNA
aatgtacagtattgcgttttgaatcgaagcgctacctgattccaattcc

SEQ ID NO: 384 - DNA, 46 bp, synthetic construct
  misc_feature 1..46: note = Primer
  source 1..46: mol_type = other DNA
aatgtacagtattgcgttttgccgaccgtaactattcggtgcgttg

SEQ ID NO: 385 - DNA, 57 bp, synthetic construct
  misc_feature 1..57: note = Primer
  source 1..57: mol_type = other DNA
aatgtacagtattgcgttttgacattctatccaagctgtgttctatcttgagaaact

SEQ ID NO: 386 - DNA, 48 bp, synthetic construct
  misc_feature 1..48: note = Primer
  source 1..48: mol_type = other DNA
aatgtacagtattgcgttttgcgagtgagggttttcgtggttcacatc

SEQ ID NO: 387 - DNA, 45 bp, synthetic construct
  misc_feature 1..45: note = Primer
  source 1..45: mol_type = other DNA
aatgtacagtattgcgttttgcgtgggtcccagtctgcagttaag

SEQ ID NO: 388 - DNA, 44 bp, synthetic construct
  misc_feature 1..44: note = Primer
  source 1..44: mol_type = other DNA
aatgtacagtattgcgttttggctcagagccgttccgagatctt

SEQ ID NO: 389 - DNA, 48 bp, synthetic construct
  misc_feature 1..48: note = Primer
  source 1..48: mol_type = other DNA
aatgtacagtattgcgttttggcgttccatctcccacttgtcgtagtt

SEQ ID NO: 390 - DNA, 49 bp, synthetic construct
  misc_feature 1..49: note = Primer
  source 1..49: mol_type = other DNA
aatgtacagtattgcgttttgctggccgagttggttcatcatcattcaa

SEQ ID NO: 391 - DNA, 48 bp, synthetic construct
  misc_feature 1..48: note = Primer
  source 1..48: mol_type = other DNA
aatgtacagtattgcgttttgtatggtgtgtcccccaactacgacaag

SEQ ID NO: 392 - DNA, 57 bp, synthetic construct
  misc_feature 1..57: note = Primer
  source 1..57: mol_type = other DNA
aatgtacagtattgcgttttgtgaaaagcacttcctgaaataatttcaccttcgttt

SEQ ID NO: 393 - DNA, 47 bp, synthetic construct
  misc_feature 1..47: note = Primer
  source 1..47: mol_type = other DNA
aatgtacagtattgcgttttgaggtactccatggctgacgagatctg

SEQ ID NO: 394 - DNA, 48 bp, synthetic construct
  misc_feature 1..48: note = Primer
  source 1..48: mol_type = other DNA
aatgtacagtattgcgttttgttgcctttgttccaaggtccaatgtgt

SEQ ID NO: 395 - DNA, 43 bp, synthetic construct
  misc_feature 1..43: note = Primer
  source 1..43: mol_type = other DNA
aatgtacagtattgcgttttgcgtccccgcattccaacgtctc

SEQ ID NO: 396 - DNA, 42 bp, synthetic construct
  misc_feature 1..42: note = Primer
  source 1..42: mol_type = other DNA
aatgtacagtattgcgttttgggcgcgccgtttacttgaagg

SEQ ID NO: 397 - DNA, 44 bp, synthetic construct
  misc_feature 1..44: note = Primer
  source 1..44: mol_type = other DNA
aatgtacagtattgcgttttggcctggcggtgcacactattctg

SEQ ID NO: 398 - DNA, 48 bp, synthetic construct
  misc_feature 1..48: note = Primer
  source 1..48: mol_type = other DNA
aatgtacagtattgcgttttgaggtgcagccacaaaacttacagatgc

SEQ ID NO: 399 - DNA, 45 bp, synthetic construct
  misc_feature 1..45: note = Primer
  source 1..45: mol_type = other DNA
aatgtacagtattgcgttttggtgccgaaccaatacaaccctctg

SEQ ID NO: 400 - DNA, 43 bp, synthetic construct
  misc_feature 1..43: note = Primer
  source 1..43: mol_type = other DNA
aatgtacagtattgcgttttggggcgggtccaccagtttgaat

SEQ ID NO: 401 - DNA, 44 bp, synthetic construct
  misc_feature 1..44: note = Primer
  source 1..44: mol_type = other DNA
aatgtacagtattgcgttttgccgcagagggttgtattggttcg

SEQ ID NO: 402 - DNA, 47 bp, synthetic construct
  misc_feature 1..47: note = Primer
  source 1..47: mol_type = other DNA
aatgtacagtattgcgttttgagccactcgcattgaccattcaaact

SEQ ID NO: 403 - DNA, 44 bp, synthetic construct
  misc_feature 1..44: note = Primer
  source 1..44: mol_type = other DNA
aatgtacagtattgcgttttgccacgtctgacaggtagccatgg

SEQ ID NO: 404 - DNA, 45 bp, synthetic construct
  misc_feature 1..45: note = Primer
  source 1..45: mol_type = other DNA
aatgtacagtattgcgttttggtgaggctgctggacgagtacaac

SEQ ID NO: 405 - DNA, 43 bp, synthetic construct
  misc_feature 1..43: note = Primer
  source 1..43: mol_type = other DNA
aatgtacagtattgcgttttgcgcaccaggttgtactcgtcca

SEQ ID NO: 406 - DNA, 46 bp, synthetic construct
  misc_feature 1..46: note = Primer
  source 1..46: mol_type = other DNA
aatgtacagtattgcgttttgccgcctttgtgcttctgttcttcgt

SEQ ID NO: 407 - DNA, 57 bp, synthetic construct
  misc_feature 1..57: note = Primer
  source 1..57: mol_type = other DNA
aatgtacagtattgcgttttgctgattaatcgcgtagaaaatgaccttattttggag

SEQ ID NO: 408 - DNA, 49 bp, synthetic construct
  misc_feature 1..49: note = Primer
  source 1..49: mol_type = other DNA
aatgtacagtattgcgttttggctccatcgtctacctggagattgacaa

SEQ ID NO: 409 - DNA, 45 bp, synthetic construct
  misc_feature 1..45: note = Primer
  source 1..45: mol_type = other DNA
aatgtacagtattgcgttttgtctgcacggcctcgatcttgtagg

SEQ ID NO: 410 - DNA, 48 bp, synthetic construct
  misc_feature 1..48: note = Primer
  source 1..48: mol_type = other DNA
aatgtacagtattgcgttttggccagcagatgatcttcccctactacg

SEQ ID NO: 411 - DNA, 44 bp, synthetic construct
  misc_feature 1..44: note = Primer
  source 1..44: mol_type = other DNA
aatgtacagtattgcgttttgcgtcacgcttgaagaccacgttg

SEQ ID NO: 412 - DNA, 45 bp, synthetic construct
  misc_feature 1..45: note = Primer
  source 1..45: mol_type = other DNA
aatgtacagtattgcgttttggccagcatgcagttctaaggctct

SEQ ID NO: 413 - DNA, 43 bp, synthetic construct
  misc_feature 1..43: note = Primer
  source 1..43: mol_type = other DNA
aatgtacagtattgcgttttggtgcccgtctcgactcttaggc

SEQ ID NO: 414 - DNA, 49 bp, synthetic construct
  misc_feature 1..49: note = Primer
  source 1..49: mol_type = other DNA
aatgtacagtattgcgttttgtgtagccgctgatcgtcgtgtatatgtc

SEQ ID NO: 415 - DNA, 57 bp, synthetic construct
  misc_feature 1..57: note = Primer
  source 1..57: mol_type = other DNA
aatgtacagtattgcgttttggactggtactggttagtaaaggttgataatattcca

SEQ ID NO: 416 - DNA, 57 bp, synthetic construct
  misc_feature 1..57: note = Primer
  source 1..57: mol_type = other DNA
aatgtacagtattgcgttttgggtgaagtaatcagtttgttcactagttacgtgatt

SEQ ID NO: 417 - DNA, 57 bp, synthetic construct
  misc_feature 1..57: note = Primer
  source 1..57: mol_type = other DNA
aatgtacagtattgcgttttgctgacatgcctactgattattcttcaaactcatcac

SEQ ID NO: 418 - DNA, 57 bp, synthetic construct
  misc_feature 1..57: note = Primer
  source 1..57: mol_type = other DNA
aatgtacagtattgcgttttgtgtgtgttttaattgttccacttgagattcttaacc

SEQ ID NO: 419 - DNA, 57 bp, synthetic construct
  misc_feature 1..57: note = Primer
  source 1..57: mol_type = other DNA
aatgtacagtattgcgttttgcgtcagcattttgaatcacttcattctgacatgata

SEQ ID NO: 420 - DNA, 57 bp, synthetic construct
  misc_feature 1..57: note = Primer
  source 1..57: mol_type = other DNA
aatgtacagtattgcgttttgagtaattttcaactattggcctagtgaatttaagct

SEQ ID NO: 421 - DNA, 55 bp, synthetic construct
  misc_feature 1..55: note = Primer
  source 1..55: mol_type = other DNA
aatgtacagtattgcgttttgagaaagagggaagtcacatttatagagtgctagc

SEQ ID NO: 422 - DNA, 57 bp, synthetic construct
  misc_feature 1..57: note = Primer
  source 1..57: mol_type = other DNA
aatgtacagtattgcgttttgcatcaacagaaacagaacaacaaactgtgacaaatc

SEQ ID NO: 423 - DNA, 57 bp, synthetic construct
  misc_feature 1..57: note = Primer
  source 1..57: mol_type = other DNA
aatgtacagtattgcgttttgccaaagaatatccctttatatagcagtggaacaatt

SEQ ID NO: 424 - DNA, 55 bp, synthetic construct
  misc_feature 1..55: note = Primer
  source 1..55: mol_type = other DNA
aatgtacagtattgcgttttgcagaatatgcagtgataagtgctgtttcatcact

SEQ ID NO: 425 - DNA, 48 bp, synthetic construct
  misc_feature 1..48: note = Primer
  source 1..48: mol_type = other DNA
aatgtacagtattgcgttttgttccccctgtgacgactacttttcctc

SEQ ID NO: 426 - DNA, 48 bp, synthetic construct
  misc_feature 1..48: note = Primer
  source 1..48: mol_type = other DNA
aatgtacagtattgcgttttgcggtccctatttcttcctctgcttcgt

SEQ ID NO: 427 - DNA, 52 bp, synthetic construct
  misc_feature 1..52: note = Primer
  source 1..52: mol_type = other DNA
aatgtacagtattgcgttttgctgaacagttctgtctctattacccgacctc

SEQ ID NO: 428 - DNA, 52 bp, synthetic construct
  misc_feature 1..52: note = Primer
  source 1..52: mol_type = other DNA
aatgtacagtattgcgttttgcgttcatagccttctatccgagtatgtagca

SEQ ID NO: 429 - DNA, 47 bp, synthetic construct
  misc_feature 1..47: note = Primer
  source 1..47: mol_type = other DNA
aatgtacagtattgcgttttgccccttctgtcctcgcaggttaatcc

SEQ ID NO: 430 - DNA, 53 bp, synthetic construct
  misc_feature 1..53: note = Primer
  source 1..53: mol_type = other DNA
aatgtacagtattgcgttttggcttccagccatttctgagatatcctcacagt

SEQ ID NO: 431 - DNA, 52 bp, synthetic construct
  misc_feature 1..52: note = Primer
  source 1..52: mol_type = other DNA
aatgtacagtattgcgttttgaccaggaggaacaaagacacatgaagatcat

SEQ ID NO: 432 - DNA, 45 bp, synthetic construct
  misc_feature 1..45: note = Primer
  source 1..45: mol_type = other DNA
aatgtacagtattgcgttttggcgcccccgagtttcttacgaatc

SEQ ID NO: 433 - DNA, 57 bp, synthetic construct
  misc_feature 1..57: note = Primer
  source 1..57: mol_type = other DNA
aatgtacagtattgcgttttgtttatacacagtttggagtttgagaatcagaagact

SEQ ID NO: 434 - DNA, 57 bp, synthetic construct
  misc_feature 1..57: note = Primer
  source 1..57: mol_type = other DNA
aatgtacagtattgcgttttgggttatctctggctgatgagattatgagtgattctc

SEQ ID NO: 435 - DNA, 51 bp, synthetic construct
  misc_feature 1..51: note = Primer
  source 1..51: mol_type = other DNA
aatgtacagtattgcgttttggccaagctagtgattgatgtgattcgctat

SEQ ID NO: 436 - DNA, 47 bp, synthetic construct
  misc_feature 1..47: note = Primer
  source 1..47: mol_type = other DNA
aatgtacagtattgcgttttgcccctcctctagtactccctgtttgt

SEQ ID NO: 437 - DNA, 51 bp, synthetic construct
  misc_feature 1..51: note = Primer
  source 1..51: mol_type = other DNA
aatgtacagtattgcgttttgctccttcctgtcccaatcaactagtctagc

SEQ ID NO: 438 - DNA, 46 bp, synthetic construct
  misc_feature 1..46: note = Primer
  source 1..46: mol_type = other DNA
aatgtacagtattgcgttttggcctcgtccctcttcccttaggtaa

SEQ ID NO: 439 - DNA, 55 bp, synthetic construct
  misc_feature 1..55: note = Primer
  source 1..55: mol_type = other DNA
aatgtacagtattgcgttttgtctctcttcccattagtctgagtactgagtgatt

SEQ ID NO: 440 - DNA, 56 bp, synthetic construct
  misc_feature 1..56: note = Primer
  source 1..56: mol_type = other DNA
aatgtacagtattgcgttttgagcatttcttgagacttaaagtggcattctaaagg

SEQ ID NO: 441 - DNA, 57 bp, synthetic construct
  misc_feature 1..57: note = Primer
  source 1..57: mol_type = other DNA
aatgtacagtattgcgttttgatttttattctcaagaggcagaaataccaacttacc

SEQ ID NO: 442 - DNA, 57 bp, synthetic construct
  misc_feature 1..57: note = Primer
  source 1..57: mol_type = other DNA
aatgtacagtattgcgttttgaatttatagctcttttcatctgctttggtatcatca

SEQ ID NO: 443 - DNA, 56 bp, synthetic construct
  misc_feature 1..56: note = Primer
  source 1..56: mol_type = other DNA
aatgtacagtattgcgttttggcctctaatctgatatacagccttagaaagtcaca

SEQ ID NO: 444 - DNA, 47 bp, synthetic construct
  misc_feature 1..47: note = Primer
  source 1..47: mol_type = other DNA
aatgtacagtattgcgttttgtgtgccattgtcctggagcaacaatt

SEQ ID NO: 445 - DNA, 57 bp, synthetic construct
  misc_feature 1..57: note = Primer
  source 1..57: mol_type = other DNA
aatgtacagtattgcgttttgagtgtactgctcgttttcttaatttgaaaagtgagt

SEQ ID NO: 446 - DNA, 57 bp, synthetic construct
  misc_feature 1..57: note = Primer
  source 1..57: mol_type = other DNA
aatgtacagtattgcgttttgacccatgaactaatacttattttgagattggtccat

SEQ ID NO: 447 - DNA, 55 bp, synthetic construct
  misc_feature 1..55: note = Primer
  source 1..55: mol_type = other DNA
aatgtacagtattgcgttttgcatggtgcaacaaaagtaagaatccaacagtttt

SEQ ID NO: 448 - DNA, 57 bp, synthetic construct
  misc_feature 1..57: note = Primer
  source 1..57: mol_type = other DNA
aatgtacagtattgcgttttgttgaaatgttaagtaagcttgaaataccgatagcat

SEQ ID NO: 449 - DNA, 51 bp, synthetic construct
  misc_feature 1..51: note = Primer
  source 1..51: mol_type = other DNA
aatgtacagtattgcgttttggggaggaagaaaatgaagcacgaggaaaac

SEQ ID NO: 450 - DNA, 57 bp, synthetic construct
  misc_feature 1..57: note = Primer
  source 1..57: mol_type = other DNA
aatgtacagtattgcgttttgatttgggatgtactctaaatttaaagcagcaaatca

SEQ ID NO: 451 - DNA, 57 bp, synthetic construct
  misc_feature 1..57: note = Primer
  source 1..57: mol_type = other DNA
aatgtacagtattgcgttttgtcaagagcagaatttggagactttgatattaaaact

SEQ ID NO: 452 - DNA, 57 bp, synthetic construct
  misc_feature 1..57: note = Primer
  source 1..57: mol_type = other DNA
aatgtacagtattgcgttttgcggttactaacatgtttagggaaatagacaactgtt

SEQ ID NO: 453 - DNA, 57 bp, synthetic construct
  misc_feature 1..57: note = Primer
  source 1..57: mol_type = other DNA
aatgtacagtattgcgttttgcctgacaacagatcccatataattaactttcatacc

SEQ ID NO: 454 - DNA, 55 bp, synthetic construct
  misc_feature 1..55: note = Primer
  source 1..55: mol_type = other DNA
aatgtacagtattgcgttttgagatgaagaagatgaggaacgagagagtaaaagc

(19) United States
(12) Patent Application Publication
(10) Pub. No.: US 2025/0257350 A1
(43) Pub. Date: Aug. 14, 2025

(54) Integrative DNA and RNA Library Preparations and Uses Thereof

(71) Applicant: QIAGEN Sciences, LLC, Germantown, MD (US)

(72) Inventors: Yexun WANG, San Diego, CA (US); Quan PENG, Clarksburg, MD (US); Daniel KIM, Brunswick, MD (US)

(21) Appl. No.: 19/006,873

(22) Filed: Dec. 31, 2024

Related U.S. Application Data
(62) Division of application No. 17/041,724, filed on Sep. 25, 2020, now Pat. No. 12,215,314.
(60) Provisional application No. 62/648,174, filed on Mar. 26, 2018.

(51) Int. Cl. C12N 15/10; C12Q 1/6806

(52) U.S. Cl. CPC C12N 15/1065; C12Q 1/6806

(57) ABSTRACT

The invention relates to integrated DNA and RNA library preparations and methods of making and uses thereof. The methods do not require physical separation of DNA and RNA. The methods output two separate libraries from DNA and RNA, respectively, which helps flexible manipulation on downstream sequencing platform.

### CROSS-REFERENCE TO RELATED APPLICATIONS

This application is a divisional of U.S. application Ser. No. 17/041,724 (now allowed), which is a 35 U.S.C. § 371 national phase of International Appl. No. PCT/US2019/024107 having an international filing date of Mar. 26, 2019, which claims benefit of U.S. Appl. No. 62/648,174, filed Mar. 26, 2018, the disclosure of each incorporated herein by reference in its entirety.

### REFERENCE TO SEQUENCE LISTING SUBMITTED ELECTRONICALLY

The content of the electronically submitted sequence listing in XML format (Name: 24950002US02SEQL.xml; Size: 585,728 bytes; and Date of Creation: Dec. 30, 2024) filed with this application is incorporated herein by reference in its entirety.

### BACKGROUND OF THE INVENTION

Paired DNA and RNA profiling enables researchers to gain more biological insights regarding the correlation between genotype and phenotype using samples from the same set of cell population.

To fully understand complex biological systems, researchers are getting more and more interested in multi-omic information, a full view of genomic, transcriptomic and proteomic data and their interactions. As the first step, paired DNA and RNA profiling are made possible with the advancement of sequencing technology. Traditional approaches, however, usually required preparing DNA and RNA samples in parallel, which meant the data obtained were not necessarily from the same set of cell population. Thus, these separate workflows might yield less correlative DNA and RNA data due to the heterogeneity of the sample. In addition, they added extra time and effort because of doubled workload.

In order to circumvent the disadvantage of separate workflow, researchers tried to integrate them. DR-seq (Dey S S et ah, Nat. Biotechnol. 33: 285-289 (2015)) and G&T-seq (Macaulay I C et ah, Nat. Methods. 72:519-22 (2015)) were among the first few attempts of integrative analysis of genomic DNA and mRNA from a single cell. However, these methods were designed specifically for single cell applications.

Another group developed an integrated DNA and RNA sequencing workflow, named Simul-seq. Reuter J A et al, Nat. Methods 75:953-958 (2016). It is a streamlined approach to profiling whole genome and transcriptome from the same set of cell population. Simul-seq is designed for whole genome and transcriptome sequencing.

There remains a need for improved, integrated DNA and RNA preparations amenable for sequencing analysis.

### BRIEF SUMMARY OF THE INVENTION

Disclosed herein are methods for preparing DNA and cDNA libraries from a sample, comprising: ligating a DNA tag to an end of a DNA molecule in a sample, wherein the DNA tag comprises a unique molecular identifier (UMI) and a DNA identifier; and performing reverse transcription of a RNA molecule in the sample in the presence of a RNA tag, wherein the RNA tag comprises a RNA identifier, a UMI, and a poly(T).

In some embodiments, the reverse transcription is performed in the presence of a second RNA tag, wherein the second RNA tag comprises a RNA identifier, a UMI, and a template switching oligonucleotide (TSO).

In some embodiments, the methods further comprise amplifying the tagged DNA and tagged cDNA for enrichment with a set of gene specific primers. In some embodiments, the methods further comprise separating the amplified sample into first and second samples.

In some embodiments, the DNA and RNA molecules are obtained from a biological sample. In some embodiments, the DNA and RNA molecules are fragmented DNA and RNA from the biological sample.

In some embodiments, the DNA molecule contains polished ends for ligation. In some embodiments, the RNA molecule is polyadenylated.

In some embodiments, the methods do not require ribosomal depletion.

In further embodiments, the methods further comprise further amplifying the first sample with primers specific for the DNA tag. The amplification can generate a DNA library corresponding to the DNA molecules in the sample.

In further embodiments, the methods further comprise further amplifying the second sample with primers specific for the RNA tag. The amplification generates a cDNA library corresponding to the RNA molecules in a sample.

In some embodiments, the methods further comprise sequencing the DNA or cDNA library. The DNA library can be used for, but not limited to, DNA variant detection, copy number analysis, fusion gene detection, or structural variant detection. The cDNA library can be used for, but not limited to, RNA variant detection, gene expression analysis, or fusion gene detection. The libraries can be also used for paired DNA and RNA profiling.

Also disclosed herein are DNA libraries made by the methods disclosed herein. Further disclosed are cDNA libraries made by the methods disclosed herein.

Also disclosed herein are DNA tags comprising a unique molecular identifier (UMI) and a DNA identifier. In some embodiments, in the DNA tags, the UMI and the DNA identifier can be positioned in a 5′ to 3′ direction.

Also disclosed herein are RNA tags comprising a RNA identifier, a UMI, and a poly(T). In some embodiments, in the RNA tags, the RNA identifier, the UMI, and the poly(T) are positioned in a 5′ to 3′ direction. Also disclosed herein are RNA tags comprising a RNA identifier, a UMI, and a template switching oligonucleotide (TSO). In some embodiments, in the RNA tags, the RNA identifier, the UMI, and the TSO are positioned in a 5′ to 3′ direction.

Disclosed herein are compositions comprising at least 2 of the above described tags. Also disclosed herein are compositions comprising the DNA tag and the 2 different RNA tags as described above.

Further disclosed herein are methods for preparing targeted DNA and cDNA libraries, comprising:
(a) obtaining purified DNA and RNA from a biological sample;
(b) fragmenting the DNA and RNA;
(c) polishing the ends of the double stranded DNA fragments for ligation;
(d) polishing the RNA fragments by polyadenylation;
(e) ligating a DNA tag to a 3′ end of the polished DNA fragments, wherein the DNA tag comprises in a 5′ to 3′ direction a unique molecular identifier (UMI) and a DNA identifier;
(f) performing reverse transcription of the polished RNA fragments in the presence of a first RNA tag, wherein the first RNA tag comprises in a 5′ to 3′ direction a RNA identifier, a UMI, and a poly(T), and a second RNA tag, wherein the second RNA tag comprises in a 5′ to 3′ direction a RNA identifier, a UMI, and a template switching oligonucleotide (TSO);
(g) amplifying the tagged DNA and tagged cDNA for enrichment with a set of gene specific primers;
(h) separating the amplified sample into first and second samples;
(i) amplifying the first sample with primers specific for the DNA tag; and
(j) amplifying the second sample with primers specific for the RNA tag.

### BRIEF DESCRIPTION OF THE DRAWINGS/FIGURES

FIG. 1. Exemplary DNA and RNA tag molecules.

FIG. 2. Exemplary process for generating DNA and cDNA libraries.

### DETAILED DESCRIPTION OF THE INVENTION

Disclosed herein are innovative approaches for integrative DNA and cDNA library preparations for analysis, such as by next-generation sequencing (NGS) analysis, without physical separation of DNA and RNA. These approaches integrate UMI (unique molecular index) technology and optionally, targeted enrichment technology, seamlessly into the workflow, which improve utilization of sequencing capacity and accuracy of the results. In addition, these methods output two separate DNA and cDNA libraries from DNA and RNA, respectively, which allow flexible manipulation on downstream sequencing platform. Compared to standalone DNA library and cDNA library methods, these approaches reduce sample consumption, simplify the experimental process, and can help researchers gain biological insights in genotype and phenotype correlations and molecular mechanisms of diseases.

Methods are described herein to prepare targeted DNA and cDNA libraries without the necessity of physical separation of genomic DNA (gDNA) and mRNA. The process involves three modules: (1) assign different DNA and RNA tag molecules to each individual DNA and RNA fragment, respectively, without separating them in the system; optionally, (2) amplify and enrich a subset of the tagged DNA and RNA fragments (target enrichment); and (3) differentially PCR amplify the tagged DNA and tagged cDNA in the (enriched) product to output two libraries corresponding to the original DNA and RNA, respectively.

The DNA and RNA tag molecules used in the first module are oligonucleotides comprising at least 1) an identifying sequence to distinguish a DNA library or RNA library, and 2) a UMI sequence for identifying each individual nucleic acid molecule.

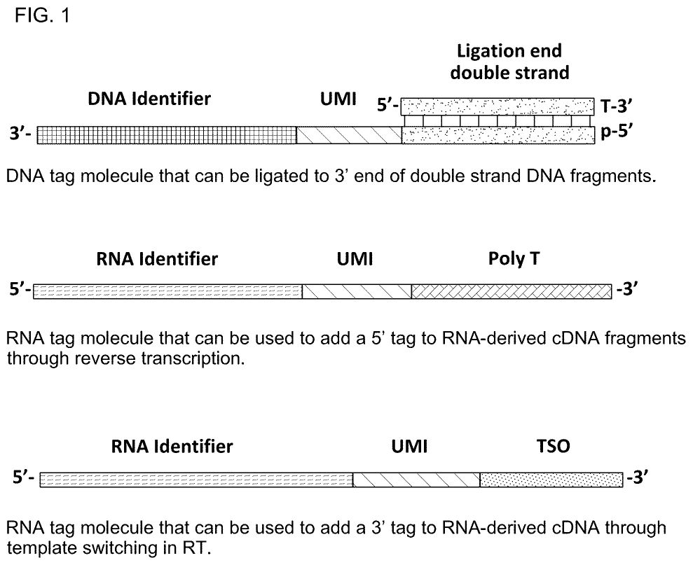

The DNA and RNA tags are essential for the final separation of DNA and cDNA libraries in module 3, where they can serve as specific amplification primer sites for DNA and RNA. The UMI sequence helps improve accuracy for both DNA and RNA NGS analysis. Exemplary tag molecules are illustrated in FIG. 1.

Two types of RNA tag molecules can be used in order to sequence the single stranded RNA from both directions, and thus, two different mechanisms can be used to attach the RNA specific sequence. Only one type of DNA tag molecule is needed because the DNA tag molecule can be ligated to both ends of the double stranded DNA.

The targeted enrichment reaction (module 2) enables focused view on relevant regions of interest and provides economic utilization of NGS sequencing capacity. It also mitigates the necessity for extra treatment of the sample associated with whole genome or transcriptome workflow, such as ribosomal RNA depletion. The enrichment is done in the same reaction for both DNA and RNA. Depending on the applications, the enrichment primer pool can be the same if the target DNA and RNA regions are the same. If different regions are of interest for the DNA and RNA, users can simply mix the corresponding enrichment primer pools, and put them into the same reaction.

Module 3 enables separated output of DNA and cDNA libraries. The sequencing depth requirements for DNA and cDNA are usually quite different, and they vary depending on the applications. The output from the methods disclosed herein gives users flexibility so that sequencing capacity can be allocated individually according to specific needs. In addition, since the samples have already been partially amplified in module 2, the separation has negligible effect on sample loss.

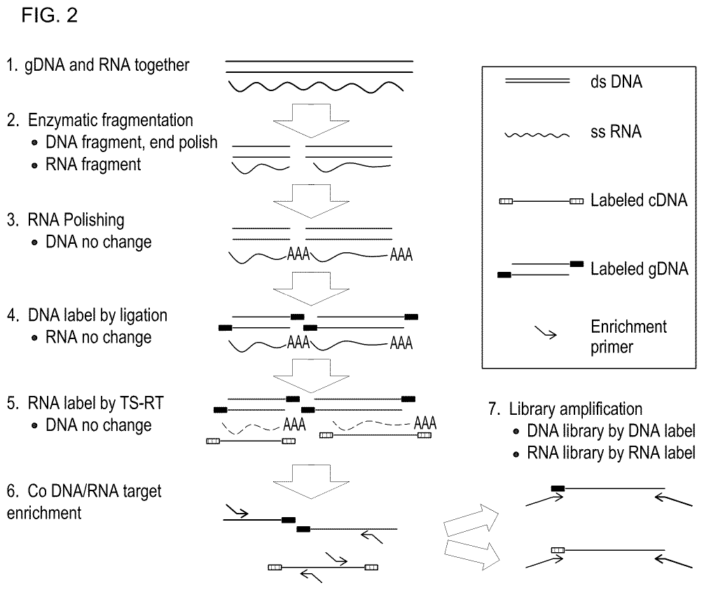

FIG. 2 illustrates one exemplary, optimized way to utilize the methods disclosed herein. It starts with purified (not necessarily separated) gDNA and RNA from a biological sample (step 1). The total nucleic acids are fragmented by enzymatic digestion (for DNA) and by heat hydrolysis (for RNA). The double stranded DNA fragments are end polished so that they are ready for ligation (step 2). The fragmented RNAs are end polished by polyadenylation (step 3). In the next few steps, DNA fragments are ligated to DNA tag molecules (step 4), and the RNA fragments are attached with RNA tag molecules (on both ends) by template switching reverse transcription (step 5). With both DNA and RNA tags in place, the sample is subjected to targeted enrichment reaction by a set of gene specific primers, in which the regions of interest are amplified and enriched (step 6). Finally, the sample is split into two samples, and further amplified by primers specific for the DNA tag and RNA tag, respectively, and with proper NGS adapter sequences compatible with, e.g., Illumina NGS platform (step 7). The final products are two separate DNA and cDNA libraries resulted from the original DNA and RNA material, respectively, and are ready for sequencing.

Disclosed herein are methods for preparing DNA and cDNA libraries from a sample, comprising: ligating a DNA tag to an end of a DNA molecule in a sample, wherein the DNA tag comprises a unique molecular identifier (UMI) and a DNA identifier; and performing reverse transcription of a RNA molecule in the sample in the presence of a RNA tag, wherein the RNA tag comprises a RNA identifier, a UMI, and a poly(T). The methods do not require physical separation of the DNA and RNA from the sample.

In some embodiments, the reverse transcription is performed in the presence of a second RNA tag, wherein the second RNA tag comprises a RNA identifier, a UMI, and a template switching oligonucleotide (TSO).

In some embodiments, the methods can include ribosomal depletion. Alternatively, in some embodiments, the methods do not require ribosomal depletion. Methods for ribosomal depletion are known in the art, e.g., using RiboZero gold (Illumina: MRZG126).

The term “sample” can include RNA, DNA, a single cell, multiple cells, fragments of cells, or an aliquot of body fluid, taken from a subject (e.g., a mammalian subject, an animal subject, a human subject, or a non-human animal subject). Samples can be selected by one of skill in the art using any known means known including but not limited to centrifugation, venipuncture, blood draw, excretion, swabbing, biopsy, needle aspirate, lavage sample, scraping, surgical incision, laser capture microdissection, gradient separation, or intervention or other means known in the art. The term “mammal” or “mammalian” as used herein includes both humans and non-humans and include but is not limited to humans, non-human primates, canines, felines, murines, bovines, equines, and porcines.

As used herein, the term “biological sample” is intended to include, but is not limited to, tissues, cells, biological fluids and isolates thereof, isolated from a subject, as well as tissues, cells, and fluids present within a subject.

As used herein, a “single cell” refers to one cell. Single cells useful in the methods described herein can be obtained from a tissue of interest, or from a biopsy, blood sample, or cell culture. Additionally, cells from specific organs, tissues, tumors, neoplasms, or the like can be obtained and used in the methods described herein. In general, cells from any population can be used in the methods, such as a population of prokaryotic or eukaryotic organisms, including bacteria or yeast.

A single cell suspension can be obtained using standard methods known in the art including, for example, enzymatically using trypsin or papain to digest proteins connecting cells in tissue samples or releasing adherent cells in culture, or mechanically separating cells in a sample. Samples can also be selected by one of skill in the art using one or more markers known to be associated with a sample of interest.

Methods for manipulating single cells are known in the art and include fluorescence activated cell sorting (FACS), micromanipulation and the use of semi-automated cell pickers (e.g., the Quixell™ cell transfer system from Stoelting Co.). Individual cells can, for example, be individually selected based on features detectable by microscopic observation, such as location, morphology, or reporter gene expression.

Once a desired sample has been identified, the sample is prepared and the cell(s) are lysed to release cellular contents including DNA and RNA, such as gDNA and mRNA, using methods known to those of skill in the art. Lysis can be achieved by, for example, heating the cells, or by the use of detergents or other chemical methods, or by a combination of these. Any suitable lysis method known in the art can be used.

Nucleic acids from a cell such as DNA or RNA are isolated using methods known to those of skill in the art.

The term “polynucleotide(s)” or “oligonucleotide(s)” refers to nucleic acids such as DNA molecules and RNA molecules and analogs thereof (e.g., DNA or RNA generated using nucleotide analogs or using nucleic acid chemistry). As desired, the polynucleotides can be made synthetically, e.g., using art-recognized nucleic acid chemistry or enzymatically using, e.g., a polymerase, and, if desired, can be modified. Typical modifications include methylation, biotinylation, and other art-known modifications. In addition, a polynucleotide can be single-stranded or double-stranded and, where desired, linked to a detectable moiety. In some aspects, a polynucleotide can include hybrid molecules, e.g., comprising DNA and RNA.

“G,” “C,” “A,” “T” and “U” each generally stands for a nucleotide that contains guanine, cytosine, adenine, thymidine and uracil as a base, respectively. However, it will be understood that the term “ribonucleotide” or “nucleotide” can also refer to a modified nucleotide or a surrogate replacement moiety. The skilled person is well aware that guanine, cytosine, adenine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in nucleotide sequences by a nucleotide containing, for example, inosine. In another example, adenine and cytosine anywhere in the oligonucleotide can be replaced with guanine and uracil, respectively, to form G-U Wobble base pairing with the target mRNA. Sequences containing such replacement moieties are suitable for the compositions and methods described herein.

The term “DNA” refers to chromosomal DNA, plasmid DNA, phage DNA, or viral DNA that is single stranded or double stranded. DNA can be obtained from prokaryotes or eukaryotes.

The term “genomic DNA” or gDNA” refers to chromosomal DNA.

The term “messenger RNA” or “mRNA” refers to an RNA that is without introns and that can be translated into a polypeptide.

The term “cDNA” refers to a DNA that is complementary or identical to an mRNA, in either single stranded or double stranded form.

As used herein, “polymerase” and its derivatives, generally refers to any enzyme that can catalyze the polymerization of nucleotides (including analogs thereof) into a nucleic acid strand. Typically. but not necessarily, such nucleotide polymerization can occur in a template-dependent fashion. Such polymerases can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze such polymerization. Optionally, the polymerase can be a mutant polymerase comprising one or more mutations involving the replacement of one or more amino acids with other amino acids, the insertion or deletion of one or more amino acids from the polymerase, or the linkage of parts of two or more polymerases. Typically, the polymerase comprises one or more active sites at which nucleotide binding and/or catalysis of nucleotide polymerization can occur. Some exemplary polymerases include without limitation DNA polymerases and RNA polymerases. The term “polymerase” and its variants, as used herein, also refers to fusion proteins comprising at least two portions linked to each other, where the first portion comprises a peptide that can catalyze the polymerization of nucleotides into a nucleic acid strand and is linked to a second portion that comprises a second polypeptide. In some embodiments, the second polypeptide can include a reporter enzyme or a processivity-enhancing domain. Optionally, the polymerase can possess 5′ exonuclease activity or terminal transferase activity. In some embodiments, the polymerase can be optionally reactivated, for example through the use of heat, chemicals or re-addition of new amounts of polymerase into a reaction mixture. In some embodiments, the polymerase can include a hot-start polymerase or an aptamer based polymerase that optionally can be reactivated.

The term “extension” and its variants, as used herein, when used in reference to a given primer, comprises any in vivo or in vitro enzymatic activity characteristic of a given polymerase that relates to polymerization of one or more nucleotides onto an end of an existing nucleic acid molecule. Typically, but not necessarily such primer extension occurs in a template-dependent fashion; during template-dependent extension, the order and selection of bases is driven by established base pairing rules, which can include Watson-Crick type base pairing rules or alternatively (and especially in the case of extension reactions involving nucleotide analogs) by some other type of base pairing paradigm. In one non-limiting example, extension occurs via polymerization of nucleotides on the 3′OH end of the nucleic acid molecule by the polymerase.

As used herein, the terms “ligating,” “ligation,” and their derivatives refer generally to the act or process for covalently linking two or more molecules together, for example, covalently linking two or more nucleic acid molecules to each other. In some embodiments, ligation includes joining nicks between adjacent nucleotides of nucleic acids. In some embodiments, ligation includes forming a covalent bond between an end of a first and an end of a second nucleic acid molecule. In some embodiments, for example embodiments wherein the nucleic acid molecules to be ligated include conventional nucleotide residues, the ligation can include forming a covalent bond between a 5′ phosphate group of one nucleic acid and a 3′ hydroxyl group of a second nucleic acid thereby forming a ligated nucleic acid molecule. In some embodiments, any means for joining nicks or bonding a 5′phosphate to a 3′ hydroxyl between adjacent nucleotides can be employed. In an exemplary embodiment, an enzyme such as a ligase can be used. Generally, for the purposes of this disclosure, an amplified target sequence can be ligated to an adapter to generate an adapter-ligated amplified target sequence.

As used herein, “ligase” and its derivatives, refers generally to any agent capable of catalyzing the ligation of two substrate molecules. In some embodiments, the ligase includes an enzyme capable of catalyzing the joining of nicks between adjacent nucleotides of a nucleic acid. In some embodiments, the ligase includes an enzyme capable of catalyzing the formation of a covalent bond between a 5′ phosphate of one nucleic acid molecule to a 3′ hydroxyl of another nucleic acid molecule thereby forming a ligated nucleic acid molecule. Suitable ligases can include, but not limited to, T4 DNA ligase, T4 RNA ligase, and E. coli DNA ligase.

As used herein, “ligation conditions” and its derivatives, generally refers to conditions suitable for ligating two molecules to each other. In some embodiments, the ligation conditions are suitable for sealing nicks or gaps between nucleic acids. As defined herein, a “nick” or “gap” refers to a nucleic acid molecule that lacks a directly bound 5′ phosphate of a mononucleotide pentose ring to a 3′ hydroxyl of a neighboring mononucleotide pentose ring within internal nucleotides of a nucleic acid sequence. As used herein, the term nick or gap is consistent with the use of the term in the art. Typically, a nick or gap can be ligated in the presence of an enzyme, such as ligase at an appropriate temperature and pH. In some embodiments, T4 DNA ligase can join a nick between nucleic acids at a temperature of about 70° C.-72° C.

As used herein, “blunt-end ligation” and its derivatives, refers generally to ligation of two blunt-end double-stranded nucleic acid molecules to each other. A “blunt end” refers to an end of a double-stranded nucleic acid molecule wherein substantially all of the nucleotides in the end of one strand of the nucleic acid molecule are base paired with opposing nucleotides in the other strand of the same nucleic acid molecule. A nucleic acid molecule is not blunt ended if it has an end that includes a single-stranded portion greater than two nucleotides in length, referred to herein as an “overhang.” In some embodiments, the end of nucleic acid molecule does not include any single stranded portion, such that every nucleotide in one strand of the end is based paired with opposing nucleotides in the other strand of the same nucleic acid molecule. In some embodiments, the ends of the two blunt ended nucleic acid molecules that become ligated to each other do not include any overlapping, shared or complementary sequence. Typically, blunted-end ligation excludes the use of additional oligonucleotide adapters to assist in the ligation of the double-stranded amplified target sequence to the double-stranded adapter, such as patch oligonucleotides as described in Mitra and Varley, US2010/0129874. In some embodiments, blunt-ended ligation includes a nick translation reaction to seal a nick created during the ligation process.

The term “amplicon” refers to the amplified product of a nucleic acid amplification reaction, e.g., RT-PCR.

The terms “reverse-transcriptase PCR” and “RT-PCR” refer to a type of PCR where the starting material is mRNA. The starting mRNA is enzymatically converted to complementary DNA or “cDNA” using a reverse transcriptase enzyme. The cDNA is then used as a template for a PCR reaction.

The terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

The term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.). Amplification methods include PCR methods known to those of skill in the art and also include rolling circle amplification (Blanco et al., J. Biol. Chem., 264, 8935-8940, 1989), hyperbranched rolling circle amplification (Lizard et al., Nat. Genetics, 19, 225-232, 1998), and loop-mediated isothermal amplification (Notomi et al., Nucl. Acids Res., 28, e63, 2000), each of which is hereby incorporated by reference in its entirety.

The term “hybridize” refers to a sequence specific non-covalent binding interaction with a complementary nucleic acid. Hybridization can occur to all or a portion of a nucleic acid sequence. Those skilled in the art will recognize that the stability of a nucleic acid duplex, or hybrids, can be determined by the Tm. Additional guidance regarding hybridization conditions can be found in: Current Protocols in Molecular Biology, John Wiley & Sons, N.Y., 1989, 6.3.1-6.3.6 and in: Sambrook et al., Molecular Cloning, a Laboratory Manual, Cold Spring Harbor Laboratory Press, Vol. 3, 1989.

As used herein, “incorporating” a sequence into a polynucleotide refers to covalently linking a series of nucleotides with the rest of the polynucleotide, for example at the 3′ or 5′ end of the polynucleotide, by phosphodiester bonds, wherein the nucleotides are linked in the order prescribed by the sequence. A sequence has been “incorporated” into a polynucleotide, or equivalently the polynucleotide “incorporates” the sequence, if the polynucleotide contains the sequence or a complement thereof. Incorporation of a sequence into a polynucleotide can occur enzymatically (e.g., by ligation or polymerization) or using chemical synthesis (e.g., by phosphoramidite chemistry).

As used herein, the terms “amplify” and “amplification” refer to enzymatically copying the sequence of a polynucleotide, in whole or in part, so as to generate more polynucleotides that also contain the sequence or a complement thereof. The sequence being copied is referred to as the template sequence. Examples of amplification include DNA-templated RNA synthesis by RNA polymerase, RNA-templated first-strand cDNA synthesis by reverse transcriptase, and DNA-templated PCR amplification using a thermostable DNA polymerase. Amplification includes all primer-extension reactions. Amplification includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., “PCR protocols: a guide to method and applications” Academic Press, Incorporated (1990) (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting amplification reaction are commercially available. Primers useful to amplify sequences from a particular gene region are preferably complementary to, and hybridize specifically to sequences in the target region or in its flanking regions and can be prepared using the polynucleotide sequences provided herein. Nucleic acid sequences generated by amplification can be sequenced directly.

The term “associated” is used herein to refer to the relationship between a sample and the DNA molecules, RNA molecules, or other polynucleotides originating from or derived from that sample. A polynucleotide is associated with a sample if it is an endogenous polynucleotide, i.e., it occurs in the sample at the time the sample is selected or is derived from an endogenous polynucleotide. For example, the mRNAs endogenous to a cell are associated with that cell. cDNAs resulting from reverse transcription of these mRNAs, and DNA amplicons resulting from PCR amplification of the cDNAs, contain the sequences of the mRNAs and are also associated with the cell. The polynucleotides associated with a sample need not be located or synthesized in the sample and are considered associated with the sample even after the sample has been destroyed (for example, after a cell has been lysed). Molecular barcoding or other techniques can be used to determine which polynucleotides in a mixture are associated with a particular sample.

When hybridization occurs in an antiparallel configuration between two single-stranded polynucleotides, the reaction is called “annealing” and those polynucleotides are described as “complementary”. As used herein, and unless otherwise indicated, the term “complementary,” when used to describe a first nucleotide sequence in relation to a second nucleotide sequence, refers to the ability of a polynucleotide comprising the first nucleotide sequence to hybridize and form a duplex structure under certain conditions with a polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C. or 70° C. for 12-16 hours followed by washing. Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

Complementary sequences include base-pairing of a region of a polynucleotide comprising a first nucleotide sequence to a region of a polynucleotide comprising a second nucleotide sequence over the length or a portion of the length of one or both nucleotide sequences. Such sequences can be referred to as “complementary” with respect to each other herein. However, where a first sequence is referred to as “substantially complementary” with respect to a second sequence herein, the two sequences can be complementary, or they can include one or more, but generally not more than about 5, 4, 3, or 2 mismatched base pairs within regions that are base-paired. For two sequences with mismatched base pairs, the sequences will be considered “substantially complementary” as long as the two nucleotide sequences bind to each other via base-pairing.

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand having the same sequence as an mRNA transcribed from that DNA and which are located 5′ to the 5′-end of the RNA transcript are referred to as “upstream sequences”; sequences on the DNA strand having the same sequence as the RNA and which are 3′ to the 3′ end of the coding RNA transcript are referred to as “downstream sequences.”

In some embodiments, the double stranded DNA fragments can be end polished so that they are amenable for ligation. For example, the ends of the DNA fragments can be polished to have blunt ends. As known in the art, this can be achieved with enzymes that can either fill in or remove the protruding strand. Another method is to perform the ligation in the presence of short synthetic oligonucleotides, called “adaptors,” which have been prepared in such a way as to eventually ligate with one terminus to the fragment and make the fragment amenable for ligation with polynucleotides of interest such as DNA or RNA tags. As such, the DNA fragments can be ligated to DNA tags.

In some embodiments, the RNA fragments are end polished by polyadenylation. The RNA fragments can be attached to RNA tags, e.g., on both ends, by template switching reverse transcription.

A “DNA tag” or “DNA tag molecule” is a polynucleotide comprising a DNA identifier and a UMI. A DNA tag can be a deoxyribopolynucleotide. A “DNA identifier” is a polynucleotide sequence assigned to distinguish a gDNA molecule from a RNA molecule. A DNA tag can be ligated to the 5′ or 3′ end of double stranded DNA fragments.

A “RNA tag” or “RNA tag molecule” is a polynucleotide comprising a RNA identifier and a UMI. A RNA tag can be a deoxyribopolynucleotide. A “RNA identifier” is a polynucleotide sequence assigned to distinguish a cDNA molecule from a gDNA molecule. A RNA tag can further comprise poly(T). Alternatively, a RNA tag can further comprise a template switching oligonucleotide (TSO). A RNA tag can be used to add a 5′ tag to RNA-derived cDNA fragments through reverse transcription. In some embodiments, a RNA tag can be used to add a 3′ tag to RNA-derived cDNA through template switching in reverse transcription.

Two types of RNA tags are helpful because in order to sequence the single stranded RNA from both directions, two different mechanisms can be used to attach the RNA specific sequence. Only one type of DNA tag is needed because the DNA tag can be ligated to both ends of the double stranded DNA.

A composition can comprise at least 2 of the tags described above, e.g., a DNA tag and a RNA tag. A composition can also comprise the 3 tags described above, e.g., a DNA tag and the 2 types of RNA tags.

Unique molecular indices or identifiers (UMIs; also called Random Molecular Tags (RMTs)) are short sequences or “barcodes” of bases used to tag each DNA or RNA molecule (fragment) prior to library amplification, thereby aiding in the identification of each individual nucleic acid molecule, or PCR duplicates. Kivioja, T. et al., Nat. Methods 9:72-74 (2012), and Suppl. If two reads align to the same location and have the same UMI, it is highly likely that they are PCR duplicates originating from the same fragment prior to amplification. UMIs can also be used to detect and quantify unique mRNA transcripts. In some embodiments, DNA tags containing the same DNA identifier sequence contain different UMI sequences. In some embodiments, RNA tags containing the same RNA identifier sequence contain different UMI sequences.

The concept of UMIs is that prior to any amplification, each original target molecule is ‘tagged’ by a unique barcode sequence. This DNA sequence must be long enough to provide sufficient permutations to assign each founder molecule a unique barcode. In some embodiments, a UMI sequence contains randomized nucleotides and is incorporated into the DNA or RNA tag. For example, a 12-base random sequence provides 4¹² or 16,777,216 UMI's for each target molecule in the sample.

In some embodiments, the RNA tag is a single-stranded DNA molecule and serves as a primer for reverse transcription. The RNA tag can be generated using a DNA polymerase (DNAP). Here, the binding site of the RNA tag is an RNA binding site (e.g., an mRNA binding site) and contains a sequence region complementary to a sequence region in one or more RNAs. In some embodiments, the binding site is complementary to a sequence region common to all RNAs in the sample to which the barcode adapter is added. For example, the binding site can be a poly(T) tract, which is complementary to the poly(A) tails of eukaryotic mRNAs. Alternatively, or in addition, the binding site can include a random sequence tract. Upon adding the RNA tag to the RNAs associated with a sample, reverse transcription can occur and first strands of cDNA can be synthesized, such that the RNA identifier sequence is incorporated into the first strands of cDNA. It will be recognized that reverse transcription requires appropriate conditions, for example the presence of an appropriate buffer and reverse transcriptase enzyme, and temperatures appropriate for annealing of the barcode adapter to RNAs and the activity of the enzyme. It will also be recognized that reverse transcription, involving a DNA primer and an RNA template, is most efficient when the 3′ end of the primer is complementary to the template and can anneal directly to the template. Accordingly, the RNA tag can be designed so that the binding site occurs at the 3′ end of the adapter molecule.

As described above, the present methods can employ a reverse transcriptase enzyme that adds one or more non-templated nucleotides (such as Cs) to the end of a nascent cDNA strand upon reaching the 5′ end of the template RNA. These nucleotides form a 3′ DNA overhang at one end of the RNA/DNA duplex. If a second RNA molecule contains a sequence region, for example, a poly-G tract at its 3′ end that is complementary to the non-templated nucleotides, and binds to the non-templated nucleotides, the reverse transcriptase can switch templates and continue extending the cDNA, now using the second RNA molecule as a template. Such a second RNA molecule is referred to herein and known in the art as a template-switching oligo (TSO).

In embodiments of the present methods, a second RNA tag comprising a RNA identifier, UMI, and TSO can serve as a template-switching oligonucleotide for reverse transcription. Thus, the RNA identifier sequence is incorporated into the first strand of cDNA after template switching and is present in DNA molecules resulting from amplification (for example, by PCR) of the first strand of cDNA. In these embodiments, any reverse transcriptase that has template switching activity can be used. The binding site of the first RNA tag is a cDNA binding site and preferably occurs at the 3′ end of the adapter molecule. The binding site can include a G-tract (comprising one or more G nucleotides), or any other sequence that is at least partially complementary to that of the 3′ overhang generated by the reverse transcriptase. It will be recognized that the overhang sequence, and thus an appropriate sequence for the binding site of the barcode adapter, can depend on the choice of reverse transcriptase used in the method.

Methods for reverse transcription and template switching are well known in the art. A procedure frequently referred to as “SMART” (switching mechanism at the 5′ end of the RNA transcript) can generate full-length cDNA libraries, even from single-cell-derived RNA samples. This strategy relies on the intrinsic properties of Moloney murine leukemia virus (MMLV) reverse transcriptase and the use of a unique template switching oligonucleotide (TS oligo, or TSO). Moloney Murine Leukemia Virus Reverse Transcriptase (M-MLV RT) is an RNA-dependent DNA polymerase that can be used in cDNA synthesis with long messenger RNA templates (>5 kb). The enzyme is a product of the pol gene of M-MLV and consists of a single subunit with a molecular weight of 71 kDa. During first-strand synthesis, upon reaching the 5′ end of the RNA template, the terminal transferase activity of the MMLV reverse transcriptase adds a few additional nucleotides (mostly deoxycytidine) to the 3′ end of the newly synthesized cDNA strand. These bases function as a TS oligo-anchoring site. Upon base pairing between the TS oligo and the appended deoxycytidine stretch, the reverse transcriptase “switches” template strands, from cellular RNA to the TS oligo, and continues replication to the 5′ end of the TS oligo. By doing so, the resulting cDNA contains the complete 5′ end of the transcript, and universal sequences of choice can be added to the reverse transcription product. Along with tagging of the cDNA 3′ end by oligo dT primers, this approach makes it possible to efficiently amplify the entire full-length transcript pool in a completely sequence-independent manner.

A TS oligo can be a DNA oligo sequence that carries 3 riboguanosines (rGrGrG) at its 3′ end. The complementarity between these consecutive rG bases and the 3′ dC extension of the cDNA molecule allows the subsequent template switching. The 3′ most rG can also be replaced with a locked nucleic acid base (LNA) to enhance thermostability of the LNA monomer, which would be advantageous for base pairing.

The TSO can include a 3′ portion comprising a plurality of guanosines or guanosine analogues that base pair with cytosine. Non-limiting examples of guanosines or guanosine analogues useful in the methods described herein include, but are not limited to, deoxyriboguanosine, riboguanosine, locked nucleic acid-guanosine, and peptide nucleic acid-guanosine. The guanosines can be ribonucleosides or locked nucleic acid monomers.

The TSO can include a 3′ portion including at least 2, at least 3, at least 4, at least 5, or 2, 3, 4, or 5, or 2-5 guanosines, or guanosine analogues that base pair with cytosine. The presence of a plurality of guanosines (or guanosine analogues that base pair with cytosine) allows the TSO to anneal transiently to the exposed cytosines at the 3′ end of the first strand of cDNA. This causes the reverse transcriptase to switch template and continue to synthesis a strand complementary to the TSO. In one aspect of the invention, the 3′ end of the TSO can be blocked, for example by a 3′ phosphate group, to prevent the TSO from functioning as a primer during cDNA synthesis.

Before the tagged cDNA samples are pooled, synthesis of cDNA can be stopped, for example by removing or inactivating the reverse transcriptase. This prevents cDNA synthesis by reverse transcription from continuing in the pooled samples.

As used herein, “amplified target sequences” and its derivatives, refers generally to a nucleic acid sequence produced by the amplification of/amplifying the target sequences using target-specific primers and the methods provided herein. The amplified target sequences can be either of the same sense (the positive strand produced in the second round and subsequent even-numbered rounds of amplification) or antisense (i.e., the negative strand produced during the first and subsequent odd-numbered rounds of amplification) with respect to the target sequences. For the purposes of this disclosure, the amplified target sequences are typically less than 50% complementary to any portion of another amplified target sequence in the reaction.

The term “polymerase chain reaction” (“PCR”) of Mullis (U.S. Pat. Nos. 4,683,195, 4,683,202, and 4,965,188) refers to a method for increasing the concentration of a segment of a target sequence in a mixture of nucleic acid sequences without cloning or purification. This process for amplifying the target sequence consists of introducing a large excess of two oligonucleotide primers to the nucleic acid sequence mixture containing the desired target sequence, followed by a precise sequence of thermal cycling in the presence of a polymerase (e.g., DNA polymerase). The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle;” there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

The methods disclosed herein can further comprise amplifying the tagged DNA the tagged cDNA for enrichment with a set of gene specific primers. Target enrichment can be achieved with, e.g., an SPE primer pool, DNA boosting primer, and RNA boosting primer. Amplicon-based next-generation sequencing (NGS) assays offer many advantages for targeted enrichment. For example, QIAseq NGS panels employ unique molecular indices (UMI's) to correct for PCR amplification bias and use single primer extension (SPE) technology which provides design flexibility and highly-specific target enrichment. The concept of UMIs is that prior to any amplification, each original target molecule is ‘tagged’ by a unique barcode sequence. This DNA sequence must be long enough to provide sufficient permutations to assign each founder molecule a unique barcode. In its current form, a 12-base random sequence provides 4¹² or 16,777,216 UMI's for each target molecule in the sample.

As used herein, the term “primer” includes an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Usually primers are extended by a DNA polymerase. Primers usually have a length in the range of between 3 to 36 nucleotides, also 5 to 24 nucleotides, also from 14 to 36 nucleotides. Primers within the scope of the invention include orthogonal primers, amplification primers, constructions primers and the like. Pairs of primers can flank a sequence of interest or a set of sequences of interest. Primers and probes can be degenerate in sequence. Primers within the scope of the present invention bind adjacent to a target sequence. A “primer” can be considered a short polynucleotide, generally with a free 3′-OH group that binds to a target or template potentially present in a sample of interest by hybridizing with the target, and thereafter promoting polymerization of a polynucleotide complementary to the target. Primers of the instant invention are comprised of nucleotides ranging from 17 to 30 nucleotides. In some embodiments, the primer is at least 17 nucleotides, or alternatively, at least 18 nucleotides, or alternatively, at least 19 nucleotides, or alternatively, at least 20 nucleotides, or alternatively, at least 21 nucleotides, or alternatively, at least 22 nucleotides, or alternatively, at least 23 nucleotides, or alternatively, at least 24 nucleotides, or alternatively, at least 25 nucleotides, or alternatively, at least 26 nucleotides, or alternatively, at least 27 nucleotides, or alternatively, at least 28 nucleotides, or alternatively, at least 29 nucleotides, or alternatively, at least 30 nucleotides, or alternatively at least 50 nucleotides, or alternatively at least 75 nucleotides or alternatively at least 100 nucleotides.

As used herein, “target-specific primer” and its derivatives, refers generally to a single stranded or double-stranded polynucleotide, typically an oligonucleotide, that includes at least one sequence that is at least 50% complementary, typically at least 75% complementary or at least 85% complementary, more typically at least 90% complementary, more typically at least 95% complementary, more typically at least 98% or at least 99% complementary, or 100% identical, to at least a portion of a nucleic acid molecule that includes a target sequence. In such instances, the target-specific primer and target sequence are described as “corresponding” to each other. In some embodiments, the target-specific primer is capable of hybridizing to at least a portion of its corresponding target sequence (or to a complement of the target sequence); such hybridization can optionally be performed under standard hybridization conditions or under stringent hybridization conditions. In some embodiments, the target-specific primer is not capable of hybridizing to the target sequence, or to its complement, but is capable of hybridizing to a portion of a nucleic acid strand including the target sequence, or to its complement. In some embodiments, the target-specific primer includes at least one sequence that is at least 75% complementary, typically at least 85% complementary, more typically at least 90% complementary, more typically at least 95% complementary, more typically at least 98% complementary, or more typically at least 99% complementary, to at least a portion of the target sequence itself; in other embodiments, the target-specific primer includes at least one sequence that is at least 75% complementary, typically at least 85% complementary, more typically at least 90% complementary, more typically at least 95% complementary, more typically at least 98% complementary, or more typically at least 99% complementary, to at least a portion of the nucleic acid molecule other than the target sequence. In some embodiments, the target-specific primer is substantially non-complementary to other target sequences present in the sample; optionally, the target-specific primer is substantially non-complementary to other nucleic acid molecules present in the sample. In some embodiments, nucleic acid molecules present in the sample that do not include or correspond to a target sequence (or to a complement of the target sequence) are referred to as “non-specific” sequences or “non-specific nucleic acids”. In some embodiments, the target-specific primer is designed to include a nucleotide sequence that is substantially complementary to at least a portion of its corresponding target sequence. In some embodiments, a target-specific primer is at least 95% complementary, or at least 99% complementary, or 100% identical, across its entire length to at least a portion of a nucleic acid molecule that includes its corresponding target sequence. In some embodiments, a target-specific primer can be at least 90%, at least 95% complementary, at least 98% complementary or at least 99% complementary, or 100% identical, across its entire length to at least a portion of its corresponding target sequence. In some embodiments, a forward target-specific primer and a reverse target-specific primer define a target-specific primer pair that can be used to amplify the target sequence via template-dependent primer extension. Typically, each primer of a target-specific primer pair includes at least one sequence that is substantially complementary to at least a portion of a nucleic acid molecule including a corresponding target sequence but that is less than 50% complementary to at least one other target sequence in the sample. In some embodiments, amplification can be performed using multiple target-specific primer pairs in a single amplification reaction, wherein each primer pair includes a forward target-specific primer and a reverse target-specific primer, each including at least one sequence that substantially complementary or substantially identical to a corresponding target sequence in the sample, and each primer pair having a different corresponding target sequence. In some embodiments, the target-specific primer can be substantially non-complementary at its 3′ end or its 5′ end to any other target-specific primer present in an amplification reaction. In some embodiments, the target-specific primer can include minimal cross hybridization to other target-specific primers in the amplification reaction. In some embodiments, target-specific primers include minimal cross-hybridization to non-specific sequences in the amplification reaction mixture. In some embodiments, the target-specific primers include minimal self-complementarity. In some embodiments, the target-specific primers can include one or more cleavable groups located at the 3′ end. In some embodiments, the target-specific primers can include one or more cleavable groups located near or about a central nucleotide of the target-specific primer. In some embodiments, one of more targets-specific primers includes only non-cleavable nucleotides at the 5′ end of the target-specific primer. In some embodiments, a target specific primer includes minimal nucleotide sequence overlap at the 3′end or the 5′ end of the primer as compared to one or more different target-specific primers, optionally in the same amplification reaction. In some embodiments 1, 2, 3, 4, 5, 6, 7, 8, 9, 10 or more, target-specific primers in a single reaction mixture include one or more of the above embodiments. In some embodiments, substantially all of the plurality of target-specific primers in a single reaction mixture includes one or more of the above embodiments.

Primer design is based on single primer extension, in which each genomic target is enriched by one target-specific primer and one universal primer—a strategy that removes conventional two target-specific primer design restriction and reduces the amount of required primers. All primers required for a panel are pooled into an individual primer pool to reduce panel handling and the number of pools required for enrichment and library construction.

The booster panel is a pool of up to 100 primers that can be used to boost the performance of certain primers in any panel (cataloged, extended, or custom), or to extend the contents of an existing custom panel. The primers are delivered as a single pool that can be spiked into the existing panel.

After removing unused adapters, a limited number of PCR cycles can be conducted using an adapter primer and a pool of single primers, each carrying a gene specific sequence and a 5′ universal sequence. During this process, each single primer repeatedly samples the same target locus from different DNA templates. Afterwards, additional PCR cycles can be conducted using universal primers to attach complete adapter sequences and to amplify the library to the desired quantity.

Compared to existing targeted enrichment approaches, the SPE method relies on single end adapter ligation, which inherently has a much higher efficiency than requiring adapters to ligate to both ends of the dsDNA fragment. More DNA molecules will be available for the downstream PCR enrichment step. PCR enrichment efficiency using one primer is also better than conventional two primer approach, due to the absence of an efficiency constraint from a second primer. During the initial PCR cycles, primers have repeated opportunities to convert (i.e. capture) maximal amount of original DNA molecules into amplicons.

All three features help to increase the efficiency of capturing rare mutations in the sample. In addition, incorporated UMI's within the amplicon are the key to estimating the number of DNA molecules captured and to greatly reduce sequencing errors in downstream analysis. Single primer extension also permits discovery of unknown structural variants, such as gene fusions.

The targeted enriched sample of DNA (e.g., gDNA) and cDNA are split into 2 separate samples. A first sample can be amplified by polymerase chain reaction (PCR) using primers specific for the DNA tag to generate a DNA library corresponding to the DNA in the sample. A second sample can be amplified by PCR using primers specific for the RNA tag to generate a cDNA library corresponding to the RNA in the sample.

A real-time polymerase chain reaction (Real-Time PCR), also known as quantitative polymerase chain reaction (qPCR), is a laboratory technique of molecular biology based on the polymerase chain reaction (PCR). It monitors the amplification of a targeted DNA molecule during the PCR, i.e. in real-time, and not at its end, as in conventional PCR. Real-time PCR can be used quantitatively (quantitative real-time PCR), and semi-quantitatively, i.e. above/below a certain amount of DNA molecules (semi quantitative real-time PCR). Other types of PCRs include but are not limited to nested PCR (used to analyze DNA sequences coming from different organisms of the same species but that can differ for a single nucleotide (SNIPS) and to ensure amplification of the sequence of interest in each of the organism analyzed) and Inverse-PCR (usually used to clone a region flanking an insert or a transposable element).

Two common methods for the detection of PCR products in real-time PCR are: (1) non-specific fluorescent dyes that intercalate with any double-stranded DNA, and (2) sequence-specific DNA probes consisting of oligonucleotides that are labeled with a fluorescent reporter which permits detection only after hybridization of the probe with its complementary sequence.

Methods and kits for performing PCR are well known in the art. PCR is a reaction in which replicate copies are made of a target polynucleotide using a pair of primers or a set of primers consisting of an upstream and a downstream primer, and a catalyst of polymerization, such as a DNA polymerase, and typically a thermally-stable polymerase enzyme. Methods for PCR are well known in the art, and taught, for example in MacPherson et al. (1991) PCR 1: A Practical Approach (IRL Press at Oxford University Press).

Embodiments of the invention provide 2 separate libraries for flexible manipulation downstream: a DNA library based on the original DNA and a cDNA library based on the original RNA produced by any of the methods described herein. The DNA library or cDNA library can be sequenced to provide an analysis of gene expression in single cells or in a plurality of single cells.

The amplified DNA or cDNA library can be sequenced and analyzed using methods known to those of skill in the art, e.g., by next-generation sequencing (NGS). In certain exemplary embodiments, RNA expression profiles are determined using any sequencing methods known in the art. Determination of the sequence of a nucleic acid sequence of interest can be performed using a variety of sequencing methods known in the art including, but not limited to, sequencing by synthesis (SBS), sequencing by hybridization (SBH), sequencing by ligation (SBL) (Shendure et al. (2005) Science 309:1728), quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), FISSEQ beads (U.S. Pat. No. 7,425,431), wobble sequencing (PCT/US05/27695), multiplex sequencing (U.S. Ser. No. 12/027,039, filed Feb. 6, 2008; Porreca et al (2007) Nat. Methods 4:931), polymerized colony (POLONY) sequencing (U.S. Pat. Nos. 6,432,360, 6,485,944 and 6,511,803, and PCT/US05/06425); nanogrid rolling circle sequencing (ROLONY) (US2009/0018024), allele-specific oligo ligation assays (e.g., oligo ligation assay (OLA), single template molecule OLA using a ligated linear probe and a rolling circle amplification (RCA) readout, ligated padlock probes, and/or single template molecule OLA using a ligated circular padlock probe and a rolling circle amplification (RCA) readout) and the like. High-throughput sequencing methods, e.g., using platforms such as Roche 454, Illumina Solexa, AB-SOLiD, Helicos, Complete Genomics, Polonator platforms and the like, can also be utilized. A variety of light-based sequencing technologies are known in the art (Landegren et al. (1998) Genome Res. 8:769-76; Kwok (2000) Pharmacogenomics 1:95-100; and Shi (2001) Clin. Chem. 47:164-172).

Embodiments of the invention also provide methods for analyzing gene expression in a plurality of single cells, the method comprising the steps of preparing a cDNA library using the method described herein and sequencing the cDNA library. A “gene” refers to a polynucleotide containing at least one open reading frame (ORF) that is capable of encoding a particular polypeptide or protein after being transcribed and translated. Any of the polynucleotide sequences described herein can be used to identify larger fragments or full-length coding sequences of the gene with which they are associated. Methods of isolating larger fragment sequences are known to those of skill in the art.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression can include splicing of the mRNA in a eukaryotic cell.

The cDNA library can be sequenced by any suitable screening method. In particular, the cDNA library can be sequenced using a high-throughput screening method, such as Applied Biosystems' SOLiD sequencing technology, or Illumina's Genome Analyzer. In one aspect of the invention, the cDNA library can be shotgun sequenced. The number of reads can be at least 10,000, at least 1 million, at least 10 million, at least 100 million, or at least 1000 million. In another aspect, the number of reads can be from 10,000 to 100,000, or alternatively from 100,000 to 1 million, or alternatively from 1 million to 10 million, or alternatively from 10 million to 100 million, or alternatively from 100 million to 1000 million. A “read” is a length of continuous nucleic acid sequence obtained by a sequencing reaction.

The DNA or gDNA library generated by the methods disclosed herein can be useful for, but not limited to, DNA variant detection, copy number analysis, fusion gene detection and structural variant detection. The cDNA library generated by the methods disclosed herein can be useful for, but not limited to, RNA variant detection, gene expression analysis, and fusion gene detection. The DNA and cDNA libraries can also be used for paired DNA and RNA profiling.

The expression profiles described herein are useful in the field of predictive medicine in which diagnostic assays, prognostic assays, pharmacogenomics, and monitoring clinical trials are used for prognostic (predictive) purposes to thereby treat an individual prophylactically. Accordingly, some embodiments relate to diagnostic assays for determining the expression profile of nucleic acid sequences (e.g., RNAs), in order to determine whether an individual is at risk of developing a disorder and/or disease. Such assays can be used for prognostic or predictive purposes to thereby prophylactically treat an individual prior to the onset of the disorder and/or disease. Accordingly, in certain exemplary embodiments, methods of diagnosing and/or prognosing one or more diseases and/or disorders using one or more of expression profiling methods described herein are provided.

Some embodiments pertain to monitoring the influence of agents (e.g., drugs or other compounds administered either to inhibit or to treat or prevent a disorder and/or disease) on the expression profile of nucleic acid sequences (e.g., RNAs) in clinical trials. Accordingly, in certain exemplary embodiments, methods of monitoring one or more diseases and/or disorders before, during and/or subsequent to treatment with one or more agents using one or more of expression profiling methods described herein are provided.

Monitoring the influence of agents (e.g., drug compounds) on the level of expression of a marker of the invention can be applied not only in basic drug screening, but also in clinical trials. For example, the effectiveness of an agent to affect an expression profile can be monitored in clinical trials of subjects receiving treatment for a disease and/or disorder associated with the expression profile. In certain exemplary embodiments, the methods for monitoring the effectiveness of treatment of a subject with an agent (e.g., an agonist, antagonist, peptidomimetic, protein, peptide, nucleic acid, small molecule, or other drug candidate) comprising the steps of (i) obtaining a pre-administration sample from a subject prior to administration of the agent; (ii) detecting one or more expression profiled in the pre-administration sample; (iii) obtaining one or more post-administration samples from the subject; (iv) detecting one or more expression profiles in the post-administration samples; (v) comparing the one or more expression profiled in the pre-administration sample with the one or more expression profiles in the post-administration sample or samples; and (vi) altering the administration of the agent to the subject accordingly.

The expression profiling methods described herein allow the quantitation of gene expression. Thus, not only tissue specificity, but also the level of expression of a variety of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the time course of expression of one or more nucleic acid sequences (e.g., genes, mRNAs and the like) in an expression profile can be monitored. This can occur in various biological contexts, as disclosed herein, for example development of a disease and/or disorder, progression of a disease and/or disorder, and processes, such as cellular alterations associated with the disease and/or disorder.

The expression profiling methods described herein are also useful for ascertaining the effect of the expression of one or more nucleic acid sequences (e.g., genes, mRNAs and the like) on the expression of other nucleic acid sequences (e.g., genes, mRNAs and the like) in the same cell or in different cells. This provides, for example, for a selection of alternate molecular targets for therapeutic intervention if the ultimate or downstream target cannot be regulated.

The expression profiling methods described herein are also useful for ascertaining differential expression patterns of one or more nucleic acid sequences (e.g., genes, mRNAs and the like) in normal and abnormal cells. This provides a battery of nucleic acid sequences (e.g., genes, mRNAs and the like) that could serve as a molecular target for diagnosis or therapeutic intervention.

### EXAMPLES

Starting Material: Purified genomic DNA and total RNA. For example, 50 ng gDNA and 50 ng total RNA was purified from THP-1 cell line. Ideally, the relative amount of gDNA and RNA should represent the content in the sample.

DNA/RNA Fragmentation:

| | uL | final conc. |
|---|---|---|
| DNA/RNA sample | X | |
| H₂O | 11.8 − x | |
| 10× Fragmentation Buffer | 2 | 1× |
| 100 mM dATP | 0.6 | 3 mM |
| Exonuclease I (20 U/uL) | 1.6 | 1.6 U/uL |
| 5× Fragmentation Enzyme Mix | 4 | 1× |

Total volume: 20 uL

Incubate in thermocycler with heated lid on for 4° C. 1 min → 32° C.
15 min → 75° C. 10 min → 80° C. 20 min → 4° C. hold

RNA Polyadenylation:

| | uL | final conc. |
|---|---|---|
| Sample from previous step | 20 | |
| H₂O | 0.5 | |
| 10 mM ATP | 1.25 | 0.5 mM |
| 10 mM 3′-dATP (blocker) | 1.25 | 0.5 mM |
| T4 Polynucleotide Kinase (10 U/uL) | 1 | 0.4 U/uL |
| E. coli Poly(A) Polymerase (5 U/uL) | 1 | 0.2 U/uL |

Total volume: 25 uL

Incubate in thermocycler with heated lid on for 4° C. 1 min →
30° C. 10 min → 4° C. hold

DNA Ligation:

| | uL | final conc. |
|---|---|---|
| Sample from previous step | 25 | |
| 5× Ligation Buffer | 10 | 1× |
| 50 uM DNA ligation Adaptor | 2.8 | 2.8 uM |
| 50% PEG-6000 | 7.2 | 7.2% |
| T4 DNA ligase (600 U/uL) | 5 | 60 U/uL |

Total volume: 50 uL

Incubate in thermocycler with heated lid OFF for 4° C. 1 min →
20° C. 15 min → 4° C. hold

Purification: Add 50 uL of ice cold water to the 50 uL sample from previous step to make 100 uL total. Do 2 rounds of 1.2× Ampure XP beads purification following manufacturer's manual with the following exceptions: 1st round elution in 52 uL water; and 2nd round elution in 13 uL water.

Reverse Transcription:

| | uL | final conc. |
|---|---|---|
| Sample from previous step | 12.87 | |
| 7.5 uM TSON10T18NV oligo | 1 | 300 nM |
| 25 uM TSON10forTS oligo | 1 | 1 uM |
| 5× SuperScript II Buffer | 5 | 1× |
| 25 mM each dNTP mix | 1 | 1 mM each |
| 0.1M DTT | 1.25 | 5 mM |
| RNase Inhibitor (40 U/uL) | 0.63 | 1 U/uL |
| 300 mM MgCl₂ | 0.5 | 6 mM |
| 150 mM MnCl₂ | 0.5 | 3 mM |
| MMLV Reverse Trancriptase RNase H− (200 U/uL) | 1.25 | 10 U/uL |

Total volume: 25 uL

Incubate in thermocycler with heated lid on for 4° C. 1 min →
25° C. 10 min → 42° C. 45 min → 70° C. 15 min → 4° C. hold

Purification: Add 75 uL of ice cold water to the 25 uL sample from previous step to make 100 uL total. Do 1 round of 1.2× Ampure XP beads purification following manufacturer's manual and elute in 16.8 uL water.

Target Enrichment:

| | uL | final conc. |
|---|---|---|
| Sample from previous step | 16.8 | |
| 5× V2 Buffer | 8 | 1× |
| 2 mM each dNTP mix | 4 | 0.2 mM each |
| 100 nM each SPE primer pool | 8 | 20 nM each |
| 10 uM DNA boosting primer | 0.8 | 400 nM |
| 10 uM RNA boosting primer | 0.8 | 400 nM |
| Hot-Star Taq Polymerase (6 U/uL) | 1.6 | 0.24 U/uL |

Total volume: 40 uL

Incubate in thermocycler with heated lid on for 95° C. 13 min →
98° C. 2 min → 8 cycles of (98° C. 15 sec → 68° C. 10 min) →
72° C. 5 min → 4° C. hold

Purification: Add 60 uL of ice cold water to the 40 uL sample from previous step to make 100 uL total. Do double size selection 0.5×/0.5× with Ampure XP beads following manufacturer's manual and elute in 22 uL water.

qPCR (real-time) to determine final amplification cycles:

| | For DNA library | | For RNA library | |
|---|---|---|---|---|
| | uL | final conc. | uL | final conc. |
| Sample from previous step | 2 | | 2 | |
| 5× V2 Buffer | 2 | 1× | 2 | 1× |
| 2 mM each dNTP mix | 1 | 0.2 mM each | 1 | 0.2 mM each |
| H₂O | 2.1 | | 2.1 | |
| 20× EveGreen Dye | 0.5 | 1× | 0.5 | 1× |
| 4 uM IL2N5RS2 Universal | 1 | 400 nM | 1 | 400 nM |
| primer | | | | |
| 4 uM DNA Universal Primer | 1 | 400 nM | 0 | 0 |
| 4 uM RNA Universal Primer | 0 | 0 | 1 | 400 nM |
| Hot-Star Taq Polymerase | 0.4 | 0.24 U/uL | 0.4 | 0.24 U/uL |
| (6 U/uL) | | | | |

| | |
|---|---|
| Total volume: | Total volume: |
| 10 uL | 10 uL |

Run on ABI 7900 real time instrument: 95° C. 13 min →
98° C. 2 min → 30 cycles of (98° C. 15 sec → 62° C. 2 min).
Record the counts for both samples

Universal PCR:

| | For DNA library | | For RNA library | |
|---|---|---|---|---|
| | uL | final conc. | uL | final conc. |
| Sample from Target | 9 | | 9 | |
| Enrichment | | | | |
| 5× V2 Buffer | 5 | 1× | 5 | 1× |
| 2 mM each dNTP mix | 2.5 | 0.2 mM each | 2.5 | 0.2 mM each |
| 4 uM IL2N5RS2 Universal | 2.5 | 400 nM | 2.5 | 400 nM |
| primer | | | | |
| 4 uM DNA Universal Primer | 2.5 | 400 nM | 0 | 0 |
| 4 uM RNA Universal Primer | 0 | 0 | 2.5 | 400 nM |
| H₂O | 2.5 | | 2.5 | |
| Hot-Star Taq Polymerase | 1 | 0.24 U/uL | 1 | 0.24 U/uL |
| (6 U/uL) | | | | |

| | |
|---|---|
| Total volume: | Total volume: |
| 25 uL | 25 uL |

Incubate in thermocycler with heated lid on for 95° C. 13 min →
98° C. 2 min → “X” cycles of (98° C. 15 sec → 62° C. 2 min) →
72° C. 5 min → 4° C. hold
(X = Ct + 4) for DNA sample and RNA sample respectively.
For example, if Ct = 19 for DNA, and 15 for RNA, then run
23 cycles for DNA, and 19 cycles for RNA

Purification: Add 75 uL of ice cold water to each of the 25 uL sample from previous step to make 100 uL total. Do 1 round of 1.2× Ampure XP beads purification following manufacturer's manual and elute in 20 uL water.

Library Quantification using Agilent Bioanalyzer High Sensitivity DNA chip: Dilute the purified libraries to 2 ng/uL. Load 1 uL of this diluted sample on the bioanalyzer. Obtain molar concentration of the libraries based on bioanalyzer's electropherogram. The libraries are ready for sequencing.

Following the workflow, with 50 ng gDNA and 50 ng total RNA input, we obtained 675 ng of DNA library and 455 ng of RNA library. The same amount of 50 ng total RNA was also used with QIAseq Targeted RNAscan Panels system from QIAGEN for comparison purpose. The same amount of 50 ng gDNA was also used with QIAseq Targeted DNA Panels system from QIAGEN for comparison purpose. The samples were then put on Illumina's MiSeq machine for sequencing.

### Results

As shown in Table 1, compared to the standalone RNA library prep workflow (QIAseq Targeted RNAscan Panels system from QIAGEN), our method achieved around 24% of its enrichment efficiency on the 1^{st} strand cDNA, and around 40% of its enrichment efficiency on the 2^{nd} strand cDNA. Since RNAscan workflow had strand bias toward the 1^{st} strand, our method had less bias and improved strand balance. The effect of enrichment efficiency on RNA analysis deserves further exploration.

TABLE 1

| Workflow | RNAScan | Ours |
|---|---|---|
| Average UMIs/ | 11061 | 2681 |
| primer 1^{st} strand | | |
| Average UMIs/ | 5279 | 2077 |
| primer 2^{nd} strand | | |
| Ratio 2^{nd}/1^{st} | 0.48 | 0.77 |

UMI per SPE primer for RNA sample: Primers were divided into two groups based on the RNA strand they detected. As shown in Table 2, compared to the standalone DNA library prep workflow (QIAseq Targeted DNA Panels system from QIAGEN), our method achieved slightly better enrichment efficiency. Both of the methods had comparable sequencing specificity and uniformity.

TABLE 2

| | Targeted | |
|---|---|---|
| Workflow | DNA Panels | Ours |
| Average UMIs/primer | 1471 | 1701 |
| Average reads/UMI | 3.4 | 3.0 |
| Overall specificity | 87% | 90% |
| (on-target reads/all reads) | | |
| Coverage uniformity (T50) | 24.9 | 21.6 |

Sequencing specs for DNA sample in both methods: Sequence coverage uniformity was measured by T50, the percentage of total sequence throughput captured by the bottom 50% of a target region. In the perfect uniform scenario, the T50 value equals to 50.

Cross talk between DNA and RNA was also evaluated since they remained in the same reaction. Using the same 50 ng of DNA and RNA from THP-1 cell line, the effective leaking signal from RNA to DNA was only 0.75% of the real DNA signal, as measured by the total UMIs of the primers detecting both RNA and DNA. In this case, only the extremely highly expressed genes might have an effect on corresponding DNA copy number analysis. However, if DNA copy number analysis was limited on intron regions, this effect should disappear. The effective leaking signal from DNA to RNA was around 3% on average by the same measurement. Since there were only a few copies of genome DNA in each cell in most cases, this kind of leaking could only affect those extremely low expressing genes (less than 0.1 copy per cell), which might be lower than the background noise level. In conclusion, our method demonstrated minimal cross talk between DNA and RNA samples which might not have any significant effect in real cases.

The DNA library prepared by our method can be used for DNA variant detection, and copy number analysis. The RNA library prepared by our method is suitable for gene expression analysis, fusion gene detection, and RNA variant detection. Multi-modal NGS panels can be developed based on our proposed method, and be used for biomarker screening, or targeted eQTL analysis.

Adaptor for ligation:

Equal molar mix and annealing of the following 2 oligos
to make double strand adaptor (DNA ligation Adaptor)

| | | |
|---|---|---|
| SEQ ID NO:1 | /5Phos/GGACTCCAATNNNNNNNNNNNNACGCTAA | PAGE |
| | GAAAGATCGGAAGAGCACACGTCTG/3ddC/ | Purified |
| SEQ ID NO:2 | ATT+GGAG+TCC*T/3Phos/ | STD |
| | | desalt |

Reverse Transcription Oligos:

| | | |
|---|---|---|
| SEQ ID NO:3 | CGACTCACTATAGGGCTGGAATTCTGACGNNNNNNN | PAGE |
| TSON10T18 | NNNACGTTTTTTTTTTTTTTTTTTNV | Purified |
| NV oligo | | |
| SEQ ID NO:4 | /5Me-isodC//iisodG//iisodG/TAATACGACTCACTATAG | PAGE |
| TSON10for | GGCTGGAATTCTGACGNNNNNNNNNNATCTGCrGrGrG | Purified |
| TS oligo | | |

Target Enrichment Oligos:

| | | |
|---|---|---|
| SEQ ID NO: 5 | AGCAGTGGTATCAACGCAGAGTCAAGC | STD |
| DNA boosting | AGAAGACGGCATACGAGATTCCGAAAC | desalt |
| primer | GTGACTGGAGTTCAGACGTGTGCTCTT | |
| | CCGATCTTTCTTAGCGT | |
| SEQ ID NO: 6 | GTGAGTGATGGTTGAGGATGTGTGCAA | STD |
| RNA boosting | GCAGAAGACGGCATACGAGATTACGTA | desalt |
| primer | CGGTGACTGGAGTTCAGACGTGTGCTC | |
| | TTCCGATCTCGACTCACTATAGGGCTG | |
| | GAATTCT | |

For each primer, the first set of underlined nucleotides is priming site for PCR amplification in Universal PCR reactions, the second set of underlined nucleotides in the middle is the sample idx (index) region, which can be replaced with respective sample index sequences, and the third set of underlined nucleotides is part of DNA or RNA identifier used for PCR amplification in target enrichment reactions.

uPCR Primers:

PAGE Purified
IL2N5RS2 Universal primer
SEQ ID NO: 7
AATGATACGGCGACCACCGAGATCTACACTCTTTCCCTACACGACGCTC
TTCCGATCTNNNNNAATGTACAGTATTGCGTTTTG
STD desalt
DNA Universal Primer
SEQ ID NO: 8
AAGCAGTGGTATCAACGCAGAGT
STD desalt
RNA Universal Primer
SEQ ID NO: 9
GTGAGTGATGGTTGAGGATGTGTG

SPE Primer Pool (equal molar mix of the following oligos):

SEQ ID NO: 10
AATGTACAGTATTGCGTTTTGAGCCCCAAGTCCTATGAGAACCTCTG
SEQ ID NO: 11
AATGTACAGTATTGCGTTTTGTGGCACCAGCGATCAGGTCCTTTAT
SEQ ID NO: 12
AATGTACAGTATTGCGTTTTGCTGAGTGGAGTCACAGCGGAGATAGT
SEQ ID NO: 13
AATGTACAGTATTGCGTTTTGTGTTCCACCAGTAACAACAGTTGAATGT
CC
SEQ ID NO: 14
AATGTACAGTATTGCGTTTTGGTGTGAGGAACATACTAGTGCTTTGCAA
GT
SEQ ID NO: 15
AATGTACAGTATTGCGTTTTGTTCAAAGTTGGGTCTGCTTCAGTCCAAAG
SEQ ID NO: 16
AATGTACAGTATTGCGTTTTGCCCCCAGCTTCTTCTCTCTGCACTAAG
SEQ ID NO: 17
AATGTACAGTATTGCGTTTTGGCCTTCCCAACATGCATTCTAACTTCTT
CC
SEQ ID NO: 18
AATGTACAGTATTGCGTTTTGCCAGCTACTCTCAAAATCAGCATCCTTT
GG
SEQ ID NO: 19
AATGTACAGTATTGCGTTTTGCCAGTCCTTCTGTGAGTCTATCCTCAGT
TC
SEQ ID NO: 20
AATGTACAGTATTGCGTTTTGAGAGCGAACCAAGAATGCCTGTTTACAG
SEQ ID NO: 21
AATGTACAGTATTGCGTTTTGGAGAGGCACGAGAACACACATCTATTCTG
SEQ ID NO: 22
AATGTACAGTATTGCGTTTTGTTCTCTTCAGAAGTTCCTTCGTCATCCTT
SEQ ID NO: 23
AATGTACAGTATTGCGTTTTGTGATGACATGCCCCATCACTAAAACAC
SEQ ID NO: 24
AATGTACAGTATTGCGTTTTGTGATAGAGACATGATGTAACCGTGGGAAT
TTCTTC
SEQ ID NO: 25
AATGTACAGTATTGCGTTTTGCGTTCTAAGAGAGTGACAGAAAGGTAAAG
AGGAG
SEQ ID NO: 26
AATGTACAGTATTGCGTTTTGATCACAAAGTATCTTTTTCTGTGGCTTAG
AAATCTT
SEQ ID NO: 27
AATGTACAGTATTGCGTTTTGTCAAATGTTAGCTCATTTTTGTTAATGGT
GGCTTTT
SEQ ID NO: 28
AATGTACAGTATTGCGTTTTGTGTCACATTATAAAGATTCAGGCAATGTT
TGTTAGT
SEQ ID NO: 29
AATGTACAGTATTGCGTTTTGAGTTTGTATGCAACATTTCTAAAGTTACC
TACTTGT
SEQ ID NO: 30
AATGTACAGTATTGCGTTTTGAAAATCTGTTTTCCAATAAATTCTCAGAT
CCAGGAA
SEQ ID NO: 31
AATGTACAGTATTGCGTTTTGCGACCCAGTTACCATAGCAATTTAGTGAA
ATAACTA
SEQ ID NO: 32
AATGTACAGTATTGCGTTTTGAGAGGCGCTATGTGTATTATTATAGCTAC
CTGTTAA
SEQ ID NO: 33
AATGTACAGTATTGCGTTTTGCGTTTTTGACAGTTTGACAGTTAAAGGCA
TTTCC
SEQ ID NO: 34
AATGTACAGTATTGCGTTTTGCTGTCCTTATTTTGGATATTTCTCCCAAT
GAAAGTA
SEQ ID NO: 35
AATGTACAGTATTGCGTTTTGGACTTTTTGCAAATGTTTAACATAGGTGA
CAGATTT
SEQ ID NO: 36
AATGTACAGTATTGCGTTTTGAAGTAGAAAATGGAAGTCTATGTGATCAA
GAAATCG
SEQ ID NO: 37
AATGTACAGTATTGCGTTTTGGGCCTCTTAAAGATCATGTTTGTTACAGT
GCTTA
SEQ ID NO: 38
AATGTACAGTATTGCGTTTTGACAAGATTGGTCAGGAAAAGAGAATTGTT
CCTATAA
SEQ ID NO: 39
AATGTACAGTATTGCGTTTTGAGACCCTGTCTCAAAAGTAAAAAGTAAGT
TAACATG
SEQ ID NO: 40
AATGTACAGTATTGCGTTTTGTCAGTGTCTTCCAAATCCTTATGTATAGC
AGCAAT
SEQ ID NO: 41
AATGTACAGTATTGCGTTTTGAGGGTCGAGGAAGCCAGTTTACATCAA
SEQ ID NO: 42
AATGTACAGTATTGCGTTTTGAACAAAAAGATATTTTCAATATTTCTGCG
CAGGTTT
SEQ ID NO: 43
AATGTACAGTATTGCGTTTTGGTCTCGACTTGAATTGCAAAAAGATGTTA
GAAAAGC
SEQ ID NO: 44
AATGTACAGTATTGCGTTTTGAAAATGTTGGCAGTCATAACATTTGAAAC
TAATGGA
SEQ ID NO: 45
AATGTACAGTATTGCGTTTTGAGCCTCAAACAGGTTGGTTTTAAATTTGA
AGTCT
SEQ ID NO: 46
AATGTACAGTATTGCGTTTTGCCTCTGTGTGTATGTTTTAACTACAAAGC
GAAACA
SEQ ID NO: 47
AATGTACAGTATTGCGTTTTGGATTCACCTGGTAATGAGGAAAACAGCTT
TAAAATC
SEQ ID NO: 48
AATGTACAGTATTGCGTTTTGAGATCTGCTGAAAAGAAATTTGTTAAAGC
ACAATT
SEQ ID NO: 49
AATGTACAGTATTGCGTTTTGCGGCATCCCCTACATCGAGACCTC
SEQ ID NO: 50
AATGTACAGTATTGCGTTTTGCAGGGAGCAGATCAAACGGGTGAAG
SEQ ID NO: 51
AATGTACAGTATTGCGTTTTGCAAGTCTTTTGAGGACATCCACCAGTAC
AG
SEQ ID NO: 52
AATGTACAGTATTGCGTTTTGACGTGCCTGTTGGACATCCTGGATA
SEQ ID NO: 53
AATGTACAGTATTGCGTTTTGCCTGTACTGGTGGATGTCCTCAAAAGACT
SEQ ID NO: 54
AATGTACAGTATTGCGTTTTGCCCTGAGGAGCGATGACGGAATATAAGC
SEQ ID NO: 55
AATGTACAGTATTGCGTTTTGGTCGTATTCGTCCACAAAATGGTTCTGGA
TC
SEQ ID NO: 56
AATGTACAGTATTGCGTTTTGTGACTGGCAATTGTGTCAACAGGTGAAAA
SEQ ID NO: 57
AATGTACAGTATTGCGTTTTGCGCCAGCTGGAGTTTGGTCATGTTT
SEQ ID NO: 58
AATGTACAGTATTGCGTTTTGAATCCCTCTCATCACAATTTCATTCCACA
ATAGTTT
SEQ ID NO: 59
AATGTACAGTATTGCGTTTTGTCAACAACAAAGAGAATCATGAAATCAAC
CCTAGC
SEQ ID NO: 60
AATGTACAGTATTGCGTTTTGGATATGGAGCCAGCGTGTTCCGATT
SEQ ID NO: 61
AATGTACAGTATTGCGTTTTGGGCGCGGAAAGTCCTCACTCTC
SEQ ID NO: 62
AATGTACAGTATTGCGTTTTGTATGGTGAGGTTCGGCGTGTTTAAACG
SEQ ID NO: 63
AATGTACAGTATTGCGTTTTGTGGTGACAAAGTTAGAAGGGTCCATGG
SEQ ID NO: 64
AATGTACAGTATTGCGTTTTGCTTCTTTACCACCCCAGATACGACGACTA
SEQ ID NO: 65
AATGTACAGTATTGCGTTTTGCGCTCGTGGTGGTAGTCGTCGTAT
SEQ ID NO: 66
AATGTACAGTATTGCGTTTTGCCAGGAGGCCCTTTCTGTTTACAACC
SEQ ID NO: 67
AATGTACAGTATTGCGTTTTGCCCACAAGCCCAAAATATTCTACTCACTT
TGC
SEQ ID NO: 68
AATGTACAGTATTGCGTTTTGATCGCCTGCATCAAGGAAAAGGTAATGG
SEQ ID NO: 69
AATGTACAGTATTGCGTTTTGCGCGTAAGGATAGCAACTGAGGTTATCAC
SEQ ID NO: 70
AATGTACAGTATTGCGTTTTGCGACCTGACGTAACCCCTTGCTTATC
SEQ ID NO: 71
AATGTACAGTATTGCGTTTTGGGAAATGCTCTCACGTAGTCTCTCATGT
CT
SEQ ID NO: 72
AATGTACAGTATTGCGTTTTGGTCATAACCCGAAGAACAATGTTGCCAC
TA
SEQ ID NO: 73
AATGTACAGTATTGCGTTTTGGTCAGCTCAGGATAAAGCACGGATGGATA
SEQ ID NO: 74
AATGTACAGTATTGCGTTTTGCTCAGGATAAAAGCTTCCTTCTTAACAAG
TTTTTCC
SEQ ID NO: 75
AATGTACAGTATTGCGTTTTGAGAGATTGTTCCCTTGCATTGACCTCTTT
TTC
SEQ ID NO: 76
AATGTACAGTATTGCGTTTTGCCCCTCACCTTTGGAATTTACAGTCTGAA
SEQ ID NO: 77
AATGTACAGTATTGCGTTTTGTAGGTTCTTCAGGTCTCTACACTCTCCTT
TAAACT
SEQ ID NO: 78
AATGTACAGTATTGCGTTTTGGAGAAGGAGTGCAATGCCAAGATTATGAT
CC
SEQ ID NO: 79
AATGTACAGTATTGCGTTTTGGACGTTCTCCATTGTATTGGCAGTAACCA
SEQ ID NO: 80
AATGTACAGTATTGCGTTTTGCACATCTCACAGGCTCTAAAGGAATTCTA
TATCCTA
SEQ ID NO: 81
AATGTACAGTATTGCGTTTTGGAGGCAAGAGGTGAGTAGTACCAATACTG
TC
SEQ ID NO: 82
AATGTACAGTATTGCGTTTTGGAGCCCCTCCGCTTACTTGTAATCTG
SEQ ID NO: 83
AATGTACAGTATTGCGTTTTGCCAGTAAAACGTATTGAGAAAAAGGTAAA
AGCGTTA
SEQ ID NO: 84
AATGTACAGTATTGCGTTTTGGCTCAGAATAAATCGTAACAATCTCAAAG
TGCATTT
SEQ ID NO: 85
AATGTACAGTATTGCGTTTTGTGAGGTGTCCACAGGGCTCAATCTTTAC
SEQ ID NO: 86
AATGTACAGTATTGCGTTTTGCCCCTTGTATCAGTAAAGGCTATATAATA
CCGAATT
SEQ ID NO: 87
AATGTACAGTATTGCGTTTTGTCATGAAGAGAGTATCATCAGCTCGTTCA
TCATC
SEQ ID NO: 88
AATGTACAGTATTGCGTTTTGTGTCCTTTCTGCCGATGTGAAATTAAAGG
TAC
SEQ ID NO: 89
AATGTACAGTATTGCGTTTTGTCGCCCCAAATAATTTCCTGCGAACA
SEQ ID NO: 90
AATGTACAGTATTGCGTTTTGCTCATACCTCCATTCCAAGCTTTCATTGT
CTC
SEQ ID NO: 91
AATGTACAGTATTGCGTTTTGCCTGCCCTTATTTTTAACAGCAGGAACGA
AT
SEQ ID NO: 92
AATGTACAGTATTGCGTTTTGTCGATAGCGAAAGTCCTCTTTGGTCAG
SEQ ID NO: 93
AATGTACAGTATTGCGTTTTGGTTAAAGACCAACCACTAACTAAGAGACT
TTCCAAG
SEQ ID NO: 94
AATGTACAGTATTGCGTTTTGAAACCTCTTCCAGTACCTTCTTCATGGTT
CT
SEQ ID NO: 95
AATGTACAGTATTGCGTTTTGTTTCCAGGTGATGTGCTCTATGAACTCC
TT
SEQ ID NO: 96
AATGTACAGTATTGCGTTTTGGGAGCGGTGCAACAGTTCAATGGT
SEQ ID NO: 97
AATGTACAGTATTGCGTTTTGCATCCGTGGATAATGTGCACCATAACC
SEQ ID NO: 98
AATGTACAGTATTGCGTTTTGTCGGAGAGCCTGGACTGTTTGAAATC
SEQ ID NO: 99
AATGTACAGTATTGCGTTTTGAAGCCAGGTCTTCCCGATGAGAGAG
SEQ ID NO: 100
AATGTACAGTATTGCGTTTTGGGCACTCCGTGGATTTCAAACAGTC
SEQ ID NO: 101
AATGTACAGTATTGCGTTTTGCAGATATCTGCTGCCCTTTTACCTTATGG
TTT
SEQ ID NO: 102
AATGTACAGTATTGCGTTTTGTGTAGACTGCTTTGGGATTACGTCTATCA
GTTG
SEQ ID NO: 103
AATGTACAGTATTGCGTTTTGGGAAAGGAGAAAAAGGAAGTGCTACCTGA
AC
SEQ ID NO: 104
AATGTACAGTATTGCGTTTTGTTTTTCTCCCTTCCTCCTTTGAACAAAC
AG
SEQ ID NO: 105
AATGTACAGTATTGCGTTTTGACAGCTTTAGGAAAATGGAATCTCTTACC
TCCTC
SEQ ID NO: 106
AATGTACAGTATTGCGTTTTGGGGTGTTATGGTCGCGTTGGATTTCTG
SEQ ID NO: 107
AATGTACAGTATTGCGTTTTGGCTACGGCGTGCAACTCACAGAAC
SEQ ID NO: 108
AATGTACAGTATTGCGTTTTGACCGACCTCTTCCAGCGCTACTT
SEQ ID NO: 109
AATGTACAGTATTGCGTTTTGCGGGCAGGGCTTACTTACCTTGG
SEQ ID NO: 110
AATGTACAGTATTGCGTTTTGTAGCTACTGCCTGCCTTCGAAGAACGAT
SEQ ID NO: 111
AATGTACAGTATTGCGTTTTGTGTGGGTGGAAAAAGATGTGGTTAAGAAA
CAAC
SEQ ID NO: 112
AATGTACAGTATTGCGTTTTGCCCCCATATAGCTTAATCTGATGGGCATC
SEQ ID NO: 113
AATGTACAGTATTGCGTTTTGGAAAGAGCATCAGGAACAAGCCTTGAGT
AC
SEQ ID NO: 114
AATGTACAGTATTGCGTTTTGTTGAGATGCCTGACAACCTTTACACCTT
TG
SEQ ID NO: 115
AATGTACAGTATTGCGTTTTGCTCTAGGGCTGAGGGAATATGCATCTCT
SEQ ID NO: 116
AATGTACAGTATTGCGTTTTGCGTACCCAGAAGACAATGGCCTAGCTAT
SEQ ID NO: 117
AATGTACAGTATTGCGTTTTGGGGCAGCACAGATTCCCTTAACCA
SEQ ID NO: 118
AATGTACAGTATTGCGTTTTGCCATACCTTGGCTATCCCCTGAAAGTTG
SEQ ID NO: 119
AATGTACAGTATTGCGTTTTGGCCCTGATGCTCATGGAGTGTTCCT
SEQ ID NO: 120
AATGTACAGTATTGCGTTTTGCCTGGTGGTTGGGAGACGACTAC
SEQ ID NO: 121
AATGTACAGTATTGCGTTTTGTGCTGACAGGACACAGAACAAGATACCT
SEQ ID NO: 122
AATGTACAGTATTGCGTTTTGGGTACAGGTATCTTGTTCTGTGTCCTGTC
AG
SEQ ID NO: 123
AATGTACAGTATTGCGTTTTGGAGTCCCGGGCTCGATTCACAG
SEQ ID NO: 124
AATGTACAGTATTGCGTTTTGCTGGTCAGAGAGGTGTGTACTGATTGTCT
SEQ ID NO: 125
AATGTACAGTATTGCGTTTTGAGGAAAGATCAATTACATTCACAAGTTCA
CACTTCT
SEQ ID NO: 126
AATGTACAGTATTGCGTTTTGCTGCACAGTTCAGAGGATATTTAAGCTCA
ATGAC
SEQ ID NO: 127
AATGTACAGTATTGCGTTTTGCACAGACCGTCATGCATTTCTGACACTC
SEQ ID NO: 128
AATGTACAGTATTGCGTTTTGAGGCTGGTACCTGCTCTTCTTCAATC
SEQ ID NO: 129
AATGTACAGTATTGCGTTTTGCGAAATCAAACAGTTGTCTATCAGAGCCT
GTC
SEQ ID NO: 130
AATGTACAGTATTGCGTTTTGACAAAAGAAAAGAAGTCATGTCTGTATGT
GGAAA
SEQ ID NO: 131
AATGTACAGTATTGCGTTTTGTCCAGGATAATACACATCACAGTAAATAA
CACTCTG
SEQ ID NO: 132
AATGTACAGTATTGCGTTTTGCATCCTCTTTGTCATCAAGCTACAGTCTT
TTTGA
SEQ ID NO: 133
AATGTACAGTATTGCGTTTTGCTCCCATTTTTGTGCATCTTTGTTGCTG
TC
SEQ ID NO: 134
AATGTACAGTATTGCGTTTTGCAGAACTGCCTATTCCTAACTGACTCATC
ATTTC
SEQ ID NO: 135
AATGTACAGTATTGCGTTTTGGAATTCTGTTTCATCGCTGAGTGACACTC
TTTT
SEQ ID NO: 136
AATGTACAGTATTGCGTTTTGTTTTTACCTTTGCTTTTACCTTTTTGTAC
TTGTGAC
SEQ ID NO: 137
AATGTACAGTATTGCGTTTTGAGAAGGAGTCTGGAATAGAAAGGCTAACA
GAA
SEQ ID NO: 138
AATGTACAGTATTGCGTTTTGCACAAGATGTGCCAAGGGAATTGTATGC
SEQ ID NO: 139
AATGTACAGTATTGCGTTTTGAAGAGTCAATAGGTCAGAGAGTTTTATGT
TCTTCCA
SEQ ID NO: 140
AATGTACAGTATTGCGTTTTGACTGATCTTCTCAAAGTCGTCATCCTTCA
GT
SEQ ID NO: 141
AATGTACAGTATTGCGTTTTGACCCTGAGAAATAATCCAATTACCTGTTA
ATCAAGG
SEQ ID NO: 142
AATGTACAGTATTGCGTTTTGAAAAGGTATTGAGTAAAATCAGTCTTCCT
TCTACCC
SEQ ID NO: 143
AATGTACAGTATTGCGTTTTGCCTTCCTCCCTCTTTCTTTCATAAAACCT
CTCTT
SEQ ID NO: 144
AATGTACAGTATTGCGTTTTGGCCAGAGCCACCCAACTCTTAAGG
SEQ ID NO: 145
AATGTACAGTATTGCGTTTTGTGGAAGAGGAATTTAATAACGAACGTTTT
AAGAGGA
SEQ ID NO: 146
AATGTACAGTATTGCGTTTTGGCATCTACTGCCGAGGATGTTCCAAG
SEQ ID NO: 147
AATGTACAGTATTGCGTTTTGCACAGTGAGCTCAAGTGCGACATCA
SEQ ID NO: 148
AATGTACAGTATTGCGTTTTGCCGACTGGCCATCTCCTCGTAG
SEQ ID NO: 149
AATGTACAGTATTGCGTTTTGGTACCAGCGCGACTACGAGGAGAT
SEQ ID NO: 150
AATGTACAGTATTGCGTTTTGTCTTTTCTGTCAAATGGAGATGATCTCTT
CTGACTC
SEQ ID NO: 151
AATGTACAGTATTGCGTTTTGGGGAGCCCATCATCTGCAAAAACATCC
SEQ ID NO: 152
AATGTACAGTATTGCGTTTTGAAGCTGAAGAAGATGTGGAAAAGTCCCAA
TG
SEQ ID NO: 153
AATGTACAGTATTGCGTTTTGGCGTGGGATGTTTTTGCAGATGATGG
SEQ ID NO: 154
AATGTACAGTATTGCGTTTTGCGACGCTGAGGACGCTATGGATG
SEQ ID NO: 155
AATGTACAGTATTGCGTTTTGGCTGAGGCGCGTCTTCGAGAAG
SEQ ID NO: 156
AATGTACAGTATTGCGTTTTGGCGCTTGTCGTGAAAGCGAACGA
SEQ ID NO: 157
AATGTACAGTATTGCGTTTTGGCTGCCCGCCCAGTTGTTACT
SEQ ID NO: 158
AATGTACAGTATTGCGTTTTGAGACTCTGGACTGATGAAGCAATTCTGA
GT
SEQ ID NO: 159
AATGTACAGTATTGCGTTTTGTCACCGGTGACACCTTAAAACCAAAGC
SEQ ID NO: 160
AATGTACAGTATTGCGTTTTGGGCTCCTTTGTACCTCCTCCATCTTGATC
SEQ ID NO: 161
AATGTACAGTATTGCGTTTTGGTCAGTTGTCTAACAATAACAAAGATCTG
CTCTTGG
SEQ ID NO: 162
AATGTACAGTATTGCGTTTTGGGTGGGCAGCAAGAAAAAGTCCAGTAAA
SEQ ID NO: 163
AATGTACAGTATTGCGTTTTGGCCAAGGCTTTCTCTGGCATGATCTTTT
SEQ ID NO: 164
AATGTACAGTATTGCGTTTTGGGATAACTTTCTCAGCATTTCCACCAGTT
TCAAG
SEQ ID NO: 165
AATGTACAGTATTGCGTTTTGTGTCCCTAAGTTGAGTAAAATGATAGAGA
ATGAGTC
SEQ ID NO: 166
AATGTACAGTATTGCGTTTTGGCTGCCAGAAATCCAGCATCCAAAATTTG
SEQ ID NO: 167
AATGTACAGTATTGCGTTTTGGTCGCTTTCTTTTCTTAGTGCCAGGAAA
CT
SEQ ID NO: 168
AATGTACAGTATTGCGTTTTGACAGTCGAGACGATTCATGAGGGAACTTC
SEQ ID NO: 169
AATGTACAGTATTGCGTTTTGGGAAAGCTCGGCGTGTTGGATAAGAAG
SEQ ID NO: 170
AATGTACAGTATTGCGTTTTGACGCCACAAGTGACTGAAAGTTGGAAG
SEQ ID NO: 171
AATGTACAGTATTGCGTTTTGTGATGGGCTGGAGATTTGGCATAGTTTTC
SEQ ID NO: 172
AATGTACAGTATTGCGTTTTGCTATGCACCCACTTTCAACACAGTTAGGT
SEQ ID NO: 173
AATGTACAGTATTGCGTTTTGGCTTGGTCAGAAGTGCTGTTGTTGTC
SEQ ID NO: 174
AATGTACAGTATTGCGTTTTGCGTGGGCCAGAAAGTTGTCCACAATG
SEQ ID NO: 175
AATGTACAGTATTGCGTTTTGGGGATATGGATTCTCGTGGTAGAAGGTGT
AA
SEQ ID NO: 176
AATGTACAGTATTGCGTTTTGCTAATCACCAAGTTCCAAGTGTTCAGAAT
CTCC
SEQ ID NO: 177
AATGTACAGTATTGCGTTTTGACCGTAATAACCAAGGTTCATCATAGGCA
TTGAT
SEQ ID NO: 178
AATGTACAGTATTGCGTTTTGTCCCAGTGGAAGTTACTATGCACCCTAT
SEQ ID NO: 179
AATGTACAGTATTGCGTTTTGTGCTTATGCTTGTGTTTGTGTTTCCTCTT
ATGG
SEQ ID NO: 180
AATGTACAGTATTGCGTTTTGGCTTCTGTTTCTCCTTATGCTTGTTCTTC
TCAC
SEQ ID NO: 181
AATGTACAGTATTGCGTTTTGCCTGAGTGGTCTTTTTGCAGGCAAAG
SEQ ID NO: 182
AATGTACAGTATTGCGTTTTGCCGGCCACAAAGCTTCTAAGAACAAC
SEQ ID NO: 183
AATGTACAGTATTGCGTTTTGGCGGTTCATCTTGAAGGCTTGGATGT
SEQ ID NO: 184
AATGTACAGTATTGCGTTTTGTTCAGTGAAATGAACCCTTCGAATGACA
AG
SEQ ID NO: 185
AATGTACAGTATTGCGTTTTGCTCCTCCTCCTCTTTGCGTTTCTTGTC
SEQ ID NO: 186
AATGTACAGTATTGCGTTTTGGCAGCAGAGAAACAAATGAAGGACAAAC
AG
SEQ ID NO: 187
AATGTACAGTATTGCGTTTTGTAAGGAGGAGGAAGAAGACAAGAAACGCA
AA
SEQ ID NO: 188
AATGTACAGTATTGCGTTTTGTAAGGCAGGTCTGTGAGCACAAAATTTGG
SEQ ID NO: 189
AATGTACAGTATTGCGTTTTGTGGAGCTGACCAGTGACAATGACC
SEQ ID NO: 190
AATGTACAGTATTGCGTTTTGGGCCAAGAAGTCGGTGGACAAGAAC
SEQ ID NO: 191
AATGTACAGTATTGCGTTTTGGCGCAGGCGGTCATTGTCACTG
SEQ ID NO: 192
AATGTACAGTATTGCGTTTTGTTGCTGTTCTTGTCCACCGACTTCTTG
SEQ ID NO: 193
AATGTACAGTATTGCGTTTTGGCAGTGCGCGATCTGGAACTG
SEQ ID NO: 194
AATGTACAGTATTGCGTTTTGCGGCGGCGACTTTGACTACCC
SEQ ID NO: 195
AATGTACAGTATTGCGTTTTGGAGCACGAGACGTCCATCGACATC
SEQ ID NO: 196
AATGTACAGTATTGCGTTTTGCGGCCAGGAACTCGTCGTTGAA
SEQ ID NO: 197
AATGTACAGTATTGCGTTTTGGCCATGCCGGGAGAACTCTAACTC
SEQ ID NO: 198
AATGTACAGTATTGCGTTTTGTGTAACCCTCCTAAGTGTTCATACGTTGT
CTTG
SEQ ID NO: 199
AATGTACAGTATTGCGTTTTGGTCTTGGTCTCTGTTATATCTTGAGTCTA
GAACAGT
SEQ ID NO: 200
AATGTACAGTATTGCGTTTTGCAGGAGAACATGGAGGCGAGAAGAAAAT
SEQ ID NO: 201
AATGTACAGTATTGCGTTTTGGGGAAAGATTGGATGCCGGGAATCAAC
SEQ ID NO: 202
AATGTACAGTATTGCGTTTTGCGGAGGCTTGATTAGGTAGGAGGTG
SEQ ID NO: 203
AATGTACAGTATTGCGTTTTGGCGGCAGCTCAACGAGAATAAACA
SEQ ID NO: 204
AATGTACAGTATTGCGTTTTGGCCCGCATCCTTACTCCGCTTATC
SEQ ID NO: 205
AATGTACAGTATTGCGTTTTGGCTGGTTTCAAGGTAAGTGGACTCTTCC
SEQ ID NO: 206
AATGTACAGTATTGCGTTTTGGGGAATGACTGACGGAGAATCCCAAC
SEQ ID NO: 207
AATGTACAGTATTGCGTTTTGCTAAGACCGAGAGCCTGTAGGAGCTTT
SEQ ID NO: 208
AATGTACAGTATTGCGTTTTGGCCGGGCTTGTCTGGTCATCT
SEQ ID NO: 209
AATGTACAGTATTGCGTTTTGCAGCTCACCTCCAAAAAGGCAAAATTCT
TG
SEQ ID NO: 210
AATGTACAGTATTGCGTTTTGGCAGGAGGCCATGATGGATTTCTTCAA
SEQ ID NO: 211
AATGTACAGTATTGCGTTTTGCATGAGTGAAAGGAAAGAGGAAATCCCAA
TCC
SEQ ID NO: 212
AATGTACAGTATTGCGTTTTGCCTATCTTCCACAGTACTTACACAACTTC
CTAAGC
SEQ ID NO: 213
AATGTACAGTATTGCGTTTTGCTCGCCGTAGACTGTCCAGGTTTT
SEQ ID NO: 214
AATGTACAGTATTGCGTTTTGCTCACCTGATCCGTGACGTTGATGTC
SEQ ID NO: 215
AATGTACAGTATTGCGTTTTGGCCCTGATGGACTCTCGGCTACT
SEQ ID NO: 216
AATGTACAGTATTGCGTTTTGGAGAAAGATCAGGAACACTTGTCCCCTAC
TAG
SEQ ID NO: 217
AATGTACAGTATTGCGTTTTGGTCCTCCACGATCTCCTCATACTCCTC
SEQ ID NO: 218
AATGTACAGTATTGCGTTTTGTCGATGGACTTGACAAGCCCGTACTT
SEQ ID NO: 219
AATGTACAGTATTGCGTTTTGCTGGACGACGAGGAGTATGAGGAGATC
SEQ ID NO: 220
AATGTACAGTATTGCGTTTTGTACCAGAAGTCCCGGCGGTGATAAG
SEQ ID NO: 221
AATGTACAGTATTGCGTTTTGGTTCACCTCTGTGTTTGACTGCCAGAAA
SEQ ID NO: 222
AATGTACAGTATTGCGTTTTGCAATGAGTATTCTCTTCATTTCAGGTCAG
TTGATTT
SEQ ID NO: 223
AATGTACAGTATTGCGTTTTGGGCTGCTTTCTTGAAGGCTATTGGGTAT
SEQ ID NO: 224
AATGTACAGTATTGCGTTTTGAGGAGACTGGAATTCTCGAATAAGGATTA
ACA
SEQ ID NO: 225
AATGTACAGTATTGCGTTTTGGCATAGTTAAAACCTGTGTTTGGTTTTGT
AGGTCTT
SEQ ID NO: 226
AATGTACAGTATTGCGTTTTGCTCTGTGTTGGCGGATACCCTTCCATA
SEQ ID NO: 227
AATGTACAGTATTGCGTTTTGGGCATTCCTTCTTTATTGCCCTTCTTAAA
AGC
SEQ ID NO: 228
AATGTACAGTATTGCGTTTTGGCTGCTGGTCTGGCTACTATGATCTCTAC
SEQ ID NO: 229
AATGTACAGTATTGCGTTTTGGCACACAGCTTTTAAGAAGGGCAATAAAG
AAG
SEQ ID NO: 230
AATGTACAGTATTGCGTTTTGTGTATGTTTAATTCTGTACATGAGCATTT
CATCAGT
SEQ ID NO: 231
AATGTACAGTATTGCGTTTTGATTTCATACCTTGCTTAATGGGTGTAGAT
ACCAAAA
SEQ ID NO: 232
AATGTACAGTATTGCGTTTTGTTGGCGTCAAATGTGCCACTATCACTC
SEQ ID NO: 233
AATGTACAGTATTGCGTTTTGTTCTCTTTCAAGCTATGATTTAGGCATAG
AGAATCG
SEQ ID NO: 234
AATGTACAGTATTGCGTTTTGCTGCAGTTGTAGGTTATAACTATCCATTT
GTCTGAA
SEQ ID NO: 235
AATGTACAGTATTGCGTTTTGCCCTAGGTCAGATCACCCAGTCAGTTAAA
AC
SEQ ID NO: 236
AATGTACAGTATTGCGTTTTGTGGTTAAAGGTCAGCCCACTTACCAGATA
TG
SEQ ID NO: 237
AATGTACAGTATTGCGTTTTGGGGTATGCTCCCCATTTAGAGGATAAGG
SEQ ID NO: 238
AATGTACAGTATTGCGTTTTGACGTCAGATCTACAGCGAACACAACTACT
SEQ ID NO: 239
AATGTACAGTATTGCGTTTTGAGTGGTGCCAGACTCACATTCAGTTCTAA
SEQ ID NO: 240
AATGTACAGTATTGCGTTTTGCTTGGCCAGTTCCTTTCTCTAATGTATCA
TCTC
SEQ ID NO: 241
AATGTACAGTATTGCGTTTTGAAGTTTTCTTGTCTAGTATCACTTTCCCT
CATAGG
SEQ ID NO: 242
AATGTACAGTATTGCGTTTTGGGGCTCAACAGATGGTATGTGTTCTCTG
SEQ ID NO: 243
AATGTACAGTATTGCGTTTTGGCTCTCGTTTCTAACAGTTCTTTGCATTG
GATA
SEQ ID NO: 244
AATGTACAGTATTGCGTTTTGGAGGTGACCTTCAAAGTCAGAGGCTGTAT
SEQ ID NO: 245
AATGTACAGTATTGCGTTTTGGAGCAACCATCCCATCTGTCCTTGTAAC
SEQ ID NO: 246
AATGTACAGTATTGCGTTTTGGGACAAGGATGAGAAACCCAATTGGAACC
SEQ ID NO: 247
AATGTACAGTATTGCGTTTTGCGGTCCGCCAAAAGATCCCAGATTC
SEQ ID NO: 248
AATGTACAGTATTGCGTTTTGGGAGGCCACTAACCCACTTGTGATG
SEQ ID NO: 249
AATGTACAGTATTGCGTTTTGTCCAGTTTCCTAGAGGATGTAATGGGATT
TGTC
SEQ ID NO: 250
AATGTACAGTATTGCGTTTTGTCACATTTGGAGATGAGAAACGAGGTGTT
CT
SEQ ID NO: 251
AATGTACAGTATTGCGTTTTGCCCTTGGCCTGTAACATTGCTCTGATC
SEQ ID NO: 252
AATGTACAGTATTGCGTTTTGCACCTCGTTTCTCATCTCCAAATGTGATC
TC
SEQ ID NO: 253
AATGTACAGTATTGCGTTTTGCCAGTAGCTTTCCTGTTCTCGGCATT
SEQ ID NO: 254
AATGTACAGTATTGCGTTTTGGCAGCGTCAAGAATGAGAAGACTTTTGTG
SEQ ID NO: 255
AATGTACAGTATTGCGTTTTGTTGCCCTTCTGGAAATTACCCCGAGA
SEQ ID NO: 256
AATGTACAGTATTGCGTTTTGAGTTCCACCAGCTTTAATTATTCCTCTAG
CTCTC
SEQ ID NO: 257
AATGTACAGTATTGCGTTTTGGTTTCCCATGGCCATAATTTATTATCTCA
CCACAA
SEQ ID NO: 258
AATGTACAGTATTGCGTTTTGGTCACGATGACTGTATTGGACCCTCAA
SEQ ID NO: 259
AATGTACAGTATTGCGTTTTGTCCAGACCTTTGCTTTAGATTGGCAATTA
TTACTG
SEQ ID NO: 260
AATGTACAGTATTGCGTTTTGCCCTAACAACACAGAAGCAAAGCGTTCT
TT
SEQ ID NO: 261
AATGTACAGTATTGCGTTTTGCGCCCTCCTACCACCTGTACTACG
SEQ ID NO: 262
AATGTACAGTATTGCGTTTTGACTATCCAGGCGCCTTCACCTACTC
SEQ ID NO: 263
AATGTACAGTATTGCGTTTTGCTCCTAGGCGGTATCATCCTGGGTAG
SEQ ID NO: 264
AATGTACAGTATTGCGTTTTGTCTGATTCTCTTCAGATACAAGGCAGAT
CC
SEQ ID NO: 265
AATGTACAGTATTGCGTTTTGGCAGATACTTGGACTTGAGTAGGCTTATT
AAACC
SEQ ID NO: 266
AATGTACAGTATTGCGTTTTGGCGGCTCTATAAAGAATTGTCCTTATTTT
CGAACTT
SEQ ID NO: 267
AATGTACAGTATTGCGTTTTGGTTCGAGGCCTTTCTCTGAGCATCAAG
SEQ ID NO: 268
AATGTACAGTATTGCGTTTTGACATCGGCAGAAACTAGATGATCAGACC
AA
SEQ ID NO: 269
AATGTACAGTATTGCGTTTTGTTTAGGAAATCCACAATACTTTTTCTGAT
CTCTTCC
SEQ ID NO: 270
AATGTACAGTATTGCGTTTTGGCCACCAACCTCATTCTGTTTTGTTCTCT
ATC
SEQ ID NO: 271
AATGTACAGTATTGCGTTTTGCTGCATTTGTCCTTTGACTGGTGTTTAG
GT
SEQ ID NO: 272
AATGTACAGTATTGCGTTTTGCTTCGACCGACAAACCTGAGGTCATTAAA
TC
SEQ ID NO: 273
AATGTACAGTATTGCGTTTTGCCCCACATCCCAAGCTAGGAAGACC
SEQ ID NO: 274
AATGTACAGTATTGCGTTTTGCGGGCCAGTACCTTGAAAGCGATG
SEQ ID NO: 275
AATGTACAGTATTGCGTTTTGCTAACTCAATCGGCTTGTTGTGATGCGT
AT
SEQ ID NO: 276
AATGTACAGTATTGCGTTTTGCCCTCCTGGACTGTTAGTAACTTAGTCT
CC
SEQ ID NO: 277
AATGTACAGTATTGCGTTTTGCCCTCCGAGCTCCGCGAAAAT
SEQ ID NO: 278
AATGTACAGTATTGCGTTTTGGTGCTAAAAAGTGTAAGAAGAAATGAGCT
AGCAAAA
SEQ ID NO: 279
AATGTACAGTATTGCGTTTTGCATATGCCTCAGTTTGAATTCCTCTCACA
AACAA
SEQ ID NO: 280
AATGTACAGTATTGCGTTTTGGGGAGAAGAAAGAGAGATGTAGGGCTAG
AG
SEQ ID NO: 281
AATGTACAGTATTGCGTTTTGGCAAGCACTTCTGTTTTTGTCTTTTCAGT
TTCG
SEQ ID NO: 282
AATGTACAGTATTGCGTTTTGTCTCTGATATACTTGGATTGGTAATTGAG
AAAGTCT
SEQ ID NO: 283
AATGTACAGTATTGCGTTTTGGTTTGATATCTTCCCAGCAAAATAATCAG
CTCTCAT
SEQ ID NO: 284
AATGTACAGTATTGCGTTTTGTAGCCAACCTCTTTTCGATGAGCTCACT
AG
SEQ ID NO: 285
AATGTACAGTATTGCGTTTTGTGGAACAGACAAACTATCGACTGAAGTT
GT
SEQ ID NO: 286
AATGTACAGTATTGCGTTTTGGAGGCTGAGTGCAAATTTGGTCTGGAA
SEQ ID NO: 287
AATGTACAGTATTGCGTTTTGGATGGTGGTGGTTGTCTCTGATGATTACC
SEQ ID NO: 288
AATGTACAGTATTGCGTTTTGGCAAGGCGAGTCCAGAACCAAGATT
SEQ ID NO: 289
AATGTACAGTATTGCGTTTTGTCAGAAGCGACTGATCCCCATCAAGT
SEQ ID NO: 290
AATGTACAGTATTGCGTTTTGCATATGGTCACATCACCTTAACTAAACCC
ATGTTT
SEQ ID NO: 291
AATGTACAGTATTGCGTTTTGTTTCTCGGTACTGTTTATTTTGAACAAAA
CCAATCC
SEQ ID NO: 292
AATGTACAGTATTGCGTTTTGCCTCCTCCCCAAATTCCAGGAACAATAT
GA
SEQ ID NO: 293
AATGTACAGTATTGCGTTTTGTGTGCGTCATTTTATTTGGGAAAATTTGA
TACTAAC
SEQ ID NO: 294
AATGTACAGTATTGCGTTTTGCATGCAGGAGAAGTCATCCCCCTTC
SEQ ID NO: 295
AATGTACAGTATTGCGTTTTGTCTGAAAACTGGTGGTTGCCTCTAGGTT
AA
SEQ ID NO: 296
AATGTACAGTATTGCGTTTTGGCCCCTTTCTTGCTCTTCTTGGACTTG
SEQ ID NO: 297
AATGTACAGTATTGCGTTTTGCCAAGCCAAGCCAAGCTGGATATTGTG
SEQ ID NO: 298
AATGTACAGTATTGCGTTTTGCACTCACATTGTGCAGCTTGTAGTAGAG
SEQ ID NO: 299
AATGTACAGTATTGCGTTTTGGCAAAGCGTCTGCATTTGAAGGAGTTT
SEQ ID NO: 300
AATGTACAGTATTGCGTTTTGCCCTCCCGAGAACTTGCCGGTTAA
SEQ ID NO: 301
AATGTACAGTATTGCGTTTTGGCTCCCCACCACAAAAACGCAAATG
SEQ ID NO: 302
AATGTACAGTATTGCGTTTTGGTGTCACTGACGGAGAGCATGAAGATG
SEQ ID NO: 303
AATGTACAGTATTGCGTTTTGCCACCCAAAGAAGTGTCTCCTGACC
SEQ ID NO: 304
AATGTACAGTATTGCGTTTTGTCCGTCAGTGACACCTGGTACTTGAC
SEQ ID NO: 305
AATGTACAGTATTGCGTTTTGCCCTAGCTCTGCCTACCCTGATCTTTC
SEQ ID NO: 306
AATGTACAGTATTGCGTTTTGACGAGGTGGACGTCTTCTTCAATCAC
SEQ ID NO: 307
AATGTACAGTATTGCGTTTTGGCCCTGCGAGTCGAGGTGATTG
SEQ ID NO: 308
AATGTACAGTATTGCGTTTTGCCATGACTCTCAGGAATTGGCCCTATACT
TAG
SEQ ID NO: 309
AATGTACAGTATTGCGTTTTGCTTGGGACCTTCATTTCTATATAACCCCT
ATCTGG
SEQ ID NO: 310
AATGTACAGTATTGCGTTTTGTGCCAGGAAACTTTTCATTGTGCCTCTC
SEQ ID NO: 311
AATGTACAGTATTGCGTTTTGGTTACCCCATGGAACTTACCAAGCACTAG
SEQ ID NO: 312
AATGTACAGTATTGCGTTTTGGTATGAAATTCGCTGGAGGGTCATTGAAT
CAAT
SEQ ID NO: 313
AATGTACAGTATTGCGTTTTGCAGGAAGGAGCACTTACGTTTTAGCATCT
TC
SEQ ID NO: 314
AATGTACAGTATTGCGTTTTGGATTTTGAGAAATTCCCTTAATATCCCCA
TGCTCAA
SEQ ID NO: 315
AATGTACAGTATTGCGTTTTGCACAACCACATGTGTCCAGTGAAAATCC
SEQ ID NO: 316
AATGTACAGTATTGCGTTTTGTGCTTTCATCAGCAGGGTTCAATCCAAA
SEQ ID NO: 317
AATGTACAGTATTGCGTTTTGCATTTACATCATCACAGAGTATTGCTTCT
ATGGAGA
SEQ ID NO: 318
AATGTACAGTATTGCGTTTTGGTGATCTCTGGATGTCGGAATATTTAGAA
ACCTCT
SEQ ID NO: 319
AATGTACAGTATTGCGTTTTGATCTTTTGAAAACAATGGTGACTACATGG
ACATGAA
SEQ ID NO: 320
AATGTACAGTATTGCGTTTTGGGTCTAAAAAGGTCTGTGTTCCTTGAACT
TACA
SEQ ID NO: 321
AATGTACAGTATTGCGTTTTGCCAGCACCAATACATTTAATTTCTTTTCT
GCAGAC
SEQ ID NO: 322
AATGTACAGTATTGCGTTTTGGCTACAGATGGCTTGATCCTGAGTCATT
TC
SEQ ID NO: 323
AATGTACAGTATTGCGTTTTGGTCAGGCCCATACCAAGGGAAAAGATC
SEQ ID NO: 324
AATGTACAGTATTGCGTTTTGACACTGAGTGATGTCTGGTCTTATGGCA
TT
SEQ ID NO: 325
AATGTACAGTATTGCGTTTTGCACTGAGCGTTTGTTAGTCCTGGTGTTTT
SEQ ID NO: 326
AATGTACAGTATTGCGTTTTGCAGATTCTCCACAATCTCACTCAGGTGGT
AAA
SEQ ID NO: 327
AATGTACAGTATTGCGTTTTGCCCCACAGCTACGAGATCATGGTGAAAT
SEQ ID NO: 328
AATGTACAGTATTGCGTTTTGTCTCTATTCATTTTTGAGGTTTGGTTGTT
AACACTT
SEQ ID NO: 329
AATGTACAGTATTGCGTTTTGGGGAGTGCACCATTATCGGGAAAATGG
SEQ ID NO: 330
AATGTACAGTATTGCGTTTTGGCTTATTCTCATTCGTTTCATCCAGGATC
TCAAAA
SEQ ID NO: 331
AATGTACAGTATTGCGTTTTGGGGCGACGAGATTAGGCTGTTATGC
SEQ ID NO: 332
AATGTACAGTATTGCGTTTTGCCCCTCTGCATTATAAGCAGTGCCAAAA
SEQ ID NO: 333
AATGTACAGTATTGCGTTTTGGCCCACATCGTTGTAAGCCTTACATTCAA
SEQ ID NO: 334
AATGTACAGTATTGCGTTTTGCCGTTTGGAAAGCTAGTGGTTCAGAGTTC
SEQ ID NO: 335
AATGTACAGTATTGCGTTTTGGAGATCCCATCCTGCCAAAGTTTGTGATT
SEQ ID NO: 336
AATGTACAGTATTGCGTTTTGGGAAAGCCCCTGTTTCATACTGACCAAAA
SEQ ID NO: 337
AATGTACAGTATTGCGTTTTGCTTTCTCCCCACAGAAACCCATGTATGA
AG
SEQ ID NO: 338
AATGTACAGTATTGCGTTTTGGTTTGCCAGTTGTGCTTTTTGCTAAAAT
GC
SEQ ID NO: 339
AATGTACAGTATTGCGTTTTGCCCTCCCACCCTCAGGACTATACCAAT
SEQ ID NO: 340
AATGTACAGTATTGCGTTTTGTGCTCGGCAGATTGGTATAGTCCTG
SEQ ID NO: 341
AATGTACAGTATTGCGTTTTGGGCATCCTCTGTCCTATCTCCCAGATACA
SEQ ID NO: 342
AATGTACAGTATTGCGTTTTGAGGTTTTATACTAAACTTACTTTGACTGG
GTTTGG
SEQ ID NO: 343
AATGTACAGTATTGCGTTTTGCCCCCAGAGGTAAGCGTCATATGG
SEQ ID NO: 344
AATGTACAGTATTGCGTTTTGGCACAGGGAAGTAGGTACTGGGAGATTG
SEQ ID NO: 345
AATGTACAGTATTGCGTTTTGAGGCCTGCAAGGTTTTAACTGGACCTA
SEQ ID NO: 346
AATGTACAGTATTGCGTTTTGCGGGAGCTGATAAGTGGTACCTGTATGT
SEQ ID NO: 347
AATGTACAGTATTGCGTTTTGGAAAAGGGTCCCAGGTAGGTCCAGTTAA
SEQ ID NO: 348
AATGTACAGTATTGCGTTTTGCTCTCGGTGTATTTCTCTACTTACCTGTA
ATAATGC
SEQ ID NO: 349
AATGTACAGTATTGCGTTTTGTTTATTGATGTCTATGAAGTGTTGTGGTT
CCTTAAC
SEQ ID NO: 350
AATGTACAGTATTGCGTTTTGCAGAAAACAAGCTGCCGCAAAGTTCTAC
SEQ ID NO: 351
AATGTACAGTATTGCGTTTTGCAGGTGTTGCGATGATGTCACTGTACG
SEQ ID NO: 352
AATGTACAGTATTGCGTTTTGTCATTTTTCATTGGACTTGTTTTGTCAGC
TTTTTGG
SEQ ID NO: 353
AATGTACAGTATTGCGTTTTGGTTAGCCCCAATATGAAAAATAAAGCTGG
TTGGA
SEQ ID NO: 354
AATGTACAGTATTGCGTTTTGCTGGTTGGAGGTTTTTGCTAAATCTGGAA
TGA
SEQ ID NO: 355
AATGTACAGTATTGCGTTTTGTTCTTTTTGACTAGAAAACTTCAGCCACT
GTGTATT
SEQ ID NO: 356
AATGTACAGTATTGCGTTTTGCATATGACCAATTGCAGATGAGCCCATTA
TTGAA
SEQ ID NO: 357
AATGTACAGTATTGCGTTTTGAGGCATAGCTGACTCATCTATGTTTGTT
CT
SEQ ID NO: 358
AATGTACAGTATTGCGTTTTGTTCCTCATTTCTTTCACTCTGACAGTATA
AAGGTAA
SEQ ID NO: 359
AATGTACAGTATTGCGTTTTGGAACTATTCCAACAGAACAAACCGATAAC
ATCA
SEQ ID NO: 360
AATGTACAGTATTGCGTTTTGTGGATAGCAAGACAATTAGAGCCCAACTT
AGT
SEQ ID NO: 361
AATGTACAGTATTGCGTTTTGCTACTCCTCCTGTCTCTTTCCACATCATC
AATT
SEQ ID NO: 362
AATGTACAGTATTGCGTTTTGAGGACCTTATGTTGTATGCTGTATAAATC
TAAAGGT
SEQ ID NO: 363
AATGTACAGTATTGCGTTTTGGTTTGTCATCTTCTATGGTAAGTATCTTT
CTGGATG
SEQ ID NO: 364
AATGTACAGTATTGCGTTTTGTGGAGGAGAAACAGATAAAAGTTGAGTAT
ACGTTTA
SEQ ID NO: 365
AATGTACAGTATTGCGTTTTGGAGGATGACGACATGTTAGTAAGCACTAC
TACT
SEQ ID NO: 366
AATGTACAGTATTGCGTTTTGATTCCACCATCATTTCCTTCTCCAAAATT
ATCATCC
SEQ ID NO: 367
AATGTACAGTATTGCGTTTTGCTCAAAAGCACTGCCTTCTCTCATTATCT
CAC
SEQ ID NO: 368
AATGTACAGTATTGCGTTTTGAATGTATTTGACCTTCTTTTAAAGTGACA
TCGATGT
SEQ ID NO: 369
AATGTACAGTATTGCGTTTTGTGATGTTCCCAACTTCTTCTCTCATGGTT
ATCTC
SEQ ID NO: 370
AATGTACAGTATTGCGTTTTGCCCTCTGATCCCTAGATAATTTATGGGTA
GCTAGA
SEQ ID NO: 371
AATGTACAGTATTGCGTTTTGCACGAAATGCAGGTTTTGGAATATGATTA
ATGTT
SEQ ID NO: 372
AATGTACAGTATTGCGTTTTGGAACAATGTTCTACGCACATTTTGTTCTC
AGTAAA
SEQ ID NO: 373
AATGTACAGTATTGCGTTTTGTCCACGCTGCTCTCTAAATTACACTCGAA
SEQ ID NO: 374
AATGTACAGTATTGCGTTTTGACGTAGAACACATTTCATTTTACTCCTCT
TTGG
SEQ ID NO: 375
AATGTACAGTATTGCGTTTTGGTCACATGAATGTAAATCAAGAAAACAGA
TGTTGTT
SEQ ID NO: 376
AATGTACAGTATTGCGTTTTGTTCTGAACTATTTATGGACAACAGTCAAA
CAACAAT
SEQ ID NO: 377
AATGTACAGTATTGCGTTTTGTGAAGCCATTGCGAGAACTTTATCCATAA
GTATTTC
SEQ ID NO: 378
AATGTACAGTATTGCGTTTTGGCCAGAGCACATGAATAAATGAGCATCC
AT
SEQ ID NO: 379
AATGTACAGTATTGCGTTTTGGGAAGCTCTCAGGGTACAAATTCTCAGAT
CAT
SEQ ID NO: 380
AATGTACAGTATTGCGTTTTGCTCAGGGTACAAATTCTCAGATCATCAGT
CCTC
SEQ ID NO: 381
AATGTACAGTATTGCGTTTTGCTCTACACAAGCTTCCTTTCCGTCATGC
SEQ ID NO: 382
AATGTACAGTATTGCGTTTTGCCCTTCAGATCTTCTCAGCATTCGAGAGA
TC
SEQ ID NO: 383
AATGTACAGTATTGCGTTTTGAATCGAAGCGCTACCTGATTCCAATTCC
SEQ ID NO: 384
AATGTACAGTATTGCGTTTTGCCGACCGTAACTATTCGGTGCGTTG
SEQ ID NO: 385
AATGTACAGTATTGCGTTTTGACATTCTATCCAAGCTGTGTTCTATCTTG
AGAAACT
SEQ ID NO: 386
AATGTACAGTATTGCGTTTTGCGAGTGAGGGTTTTCGTGGTTCACATC
SEQ ID NO: 387
AATGTACAGTATTGCGTTTTGCGTGGGTCCCAGTCTGCAGTTAAG
SEQ ID NO: 388
AATGTACAGTATTGCGTTTTGGCTCAGAGCCGTTCCGAGATCTT
SEQ ID NO: 389
AATGTACAGTATTGCGTTTTGGCGTTCCATCTCCCACTTGTCGTAGTT
SEQ ID NO: 390
AATGTACAGTATTGCGTTTTGCTGGCCGAGTTGGTTCATCATCATTCAA
SEQ ID NO: 391
AATGTACAGTATTGCGTTTTGTATGGTGTGTCCCCCAACTACGACAAG
SEQ ID NO: 392
AATGTACAGTATTGCGTTTTGTGAAAAGCACTTCCTGAAATAATTTCACC
TTCGTTT
SEQ ID NO: 393
AATGTACAGTATTGCGTTTTGAGGTACTCCATGGCTGACGAGATCTG
SEQ ID NO: 394
AATGTACAGTATTGCGTTTTGTTGCCTTTGTTCCAAGGTCCAATGTGT
SEQ ID NO: 395
AATGTACAGTATTGCGTTTTGCGTCCCCGCATTCCAACGTCTC
SEQ ID NO: 396
AATGTACAGTATTGCGTTTTGGGCGCGCCGTTTACTTGAAGG
SEQ ID NO: 397
AATGTACAGTATTGCGTTTTGGCCTGGCGGTGCACACTATTCTG
SEQ ID NO: 398
AATGTACAGTATTGCGTTTTGAGGTGCAGCCACAAAACTTACAGATGC
SEQ ID NO: 399
AATGTACAGTATTGCGTTTTGGTGCCGAACCAATACAACCCTCTG
SEQ ID NO: 400
AATGTACAGTATTGCGTTTTGGGGCGGGTCCACCAGTTTGAAT
SEQ ID NO: 401
AATGTACAGTATTGCGTTTTGCCGCAGAGGGTTGTATTGGTTCG
SEQ ID NO: 402
AATGTACAGTATTGCGTTTTGAGCCACTCGCATTGACCATTCAAACT
SEQ ID NO: 403
AATGTACAGTATTGCGTTTTGCCACGTCTGACAGGTAGCCATGG
SEQ ID NO: 404
AATGTACAGTATTGCGTTTTGGTGAGGCTGCTGGACGAGTACAAC
SEQ ID NO: 405
AATGTACAGTATTGCGTTTTGCGCACCAGGTTGTACTCGTCCA
SEQ ID NO: 406
AATGTACAGTATTGCGTTTTGCCGCCTTTGTGCTTCTGTTCTTCGT
SEQ ID NO: 407
AATGTACAGTATTGCGTTTTGCTGATTAATCGCGTAGAAAATGACCTTAT
TTTGGAG
SEQ ID NO: 408
AATGTACAGTATTGCGTTTTGGCTCCATCGTCTACCTGGAGATTGACAA
SEQ ID NO: 409
AATGTACAGTATTGCGTTTTGTCTGCACGGCCTCGATCTTGTAGG
SEQ ID NO: 410
AATGTACAGTATTGCGTTTTGGCCAGCAGATGATCTTCCCCTACTACG
SEQ ID NO: 411
AATGTACAGTATTGCGTTTTGCGTCACGCTTGAAGACCACGTTG
SEQ ID NO: 412
AATGTACAGTATTGCGTTTTGGCCAGCATGCAGTTCTAAGGCTCT
SEQ ID NO: 413
AATGTACAGTATTGCGTTTTGGTGCCCGTCTCGACTCTTAGGC
SEQ ID NO: 414
AATGTACAGTATTGCGTTTTGTGTAGCCGCTGATCGTCGTGTATATGTC
SEQ ID NO: 415
AATGTACAGTATTGCGTTTTGGACTGGTACTGGTTAGTAAAGGTTGATAA
TATTCCA
SEQ ID NO: 416
AATGTACAGTATTGCGTTTTGGGTGAAGTAATCAGTTTGTTCACTAGTTA
CGTGATT
SEQ ID NO: 417
AATGTACAGTATTGCGTTTTGCTGACATGCCTACTGATTATTCTTCAAAC
TCATCAC
SEQ ID NO: 418
AATGTACAGTATTGCGTTTTGTGTGTGTTTTAATTGTTCCACTTGAGATT
CTTAACC
SEQ ID NO: 419
AATGTACAGTATTGCGTTTTGCGTCAGCATTTTGAATCACTTCATTCTGA
CATGATA
SEQ ID NO: 420
AATGTACAGTATTGCGTTTTGAGTAATTTTCAACTATTGGCCTAGTGAAT
TTAAGCT
SEQ ID NO: 421
AATGTACAGTATTGCGTTTTGAGAAAGAGGGAAGTCACATTTATAGAGTG
CTAGC
SEQ ID NO: 422
AATGTACAGTATTGCGTTTTGCATCAACAGAAACAGAACAACAAACTGTG
ACAAATC
SEQ ID NO: 423
AATGTACAGTATTGCGTTTTGCCAAAGAATATCCCTTTATATAGCAGTGG
AACAATT
SEQ ID NO: 424
AATGTACAGTATTGCGTTTTGCAGAATATGCAGTGATAAGTGCTGTTTCA
TCACT
SEQ ID NO: 425
AATGTACAGTATTGCGTTTTGTTCCCCCTGTGACGACTACTTTTCCTC
SEQ ID NO: 426
AATGTACAGTATTGCGTTTTGCGGTCCCTATTTCTTCCTCTGCTTCGT
SEQ ID NO: 427
AATGTACAGTATTGCGTTTTGCTGAACAGTTCTGTCTCTATTACCCGACC
TC
SEQ ID NO: 428
AATGTACAGTATTGCGTTTTGCGTTCATAGCCTTCTATCCGAGTATGTAG
CA
SEQ ID NO: 429
AATGTACAGTATTGCGTTTTGCCCCTTCTGTCCTCGCAGGTTAATCC
SEQ ID NO: 430
AATGTACAGTATTGCGTTTTGGCTTCCAGCCATTTCTGAGATATCCTCAC
AGT
SEQ ID NO: 431
AATGTACAGTATTGCGTTTTGACCAGGAGGAACAAAGACACATGAAGATC
AT
SEQ ID NO: 432
AATGTACAGTATTGCGTTTTGGCGCCCCCGAGTTTCTTACGAATC
SEQ ID NO: 433
AATGTACAGTATTGCGTTTTGTTTATACACAGTTTGGAGTTTGAGAATCA
GAAGACT
SEQ ID NO: 434
AATGTACAGTATTGCGTTTTGGGTTATCTCTGGCTGATGAGATTATGAGT
GATTCTC
SEQ ID NO: 435
AATGTACAGTATTGCGTTTTGGCCAAGCTAGTGATTGATGTGATTCGCT
AT
SEQ ID NO: 436
AATGTACAGTATTGCGTTTTGCCCCTCCTCTAGTACTCCCTGTTTGT
SEQ ID NO: 437
AATGTACAGTATTGCGTTTTGCTCCTTCCTGTCCCAATCAACTAGTCTA
GC
SEQ ID NO: 438
AATGTACAGTATTGCGTTTTGGCCTCGTCCCTCTTCCCTTAGGTAA
SEQ ID NO: 439
AATGTACAGTATTGCGTTTTGTCTCTCTTCCCATTAGTCTGAGTACTGAG
TGATT
SEQ ID NO: 440
AATGTACAGTATTGCGTTTTGAGCATTTCTTGAGACTTAAAGTGGCATTC
TAAAGG
SEQ ID NO: 441
AATGTACAGTATTGCGTTTTGATTTTTATTCTCAAGAGGCAGAAATACCA
ACTTACC
SEQ ID NO: 442
AATGTACAGTATTGCGTTTTGAATTTATAGCTCTTTTCATCTGCTTTGGT
ATCATCA
SEQ ID NO: 443
AATGTACAGTATTGCGTTTTGGCCTCTAATCTGATATACAGCCTTAGAAA
GTCACA
SEQ ID NO: 444
AATGTACAGTATTGCGTTTTGTGTGCCATTGTCCTGGAGCAACAATT
SEQ ID NO: 445
AATGTACAGTATTGCGTTTTGAGTGTACTGCTCGTTTTCTTAATTTGAAA
AGTGAGT
SEQ ID NO: 446
AATGTACAGTATTGCGTTTTGACCCATGAACTAATACTTATTTTGAGATT
GGTCCAT
SEQ ID NO: 447
AATGTACAGTATTGCGTTTTGCATGGTGCAACAAAAGTAAGAATCCAACA
GTTTT
SEQ ID NO: 448
AATGTACAGTATTGCGTTTTGTTGAAATGTTAAGTAAGCTTGAAATACCG
ATAGCAT
SEQ ID NO: 449
AATGTACAGTATTGCGTTTTGGGGAGGAAGAAAATGAAGCACGAGGAAA
AC
SEQ ID NO: 450
AATGTACAGTATTGCGTTTTGATTTGGGATGTACTCTAAATTTAAAGCAG
CAAATCA
SEQ ID NO: 451
AATGTACAGTATTGCGTTTTGTCAAGAGCAGAATTTGGAGACTTTGATAT
TAAAACT
SEQ ID NO: 452
AATGTACAGTATTGCGTTTTGCGGTTACTAACATGTTTAGGGAAATAGAC
AACTGTT
SEQ ID NO: 453
AATGTACAGTATTGCGTTTTGCCTGACAACAGATCCCATATAATTAACTT
TCATACC
SEQ ID NO: 454
AATGTACAGTATTGCGTTTTGAGATGAAGAAGATGAGGAACGAGAGAGTA
AAAGC

The foregoing description of the specific embodiments will so fully reveal the general nature of the invention that others can, by applying knowledge within the skill of the art, readily modify and/or adapt for various applications, without departing from the general concept of the invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments but should be defined only in accordance with the following claims and their equivalents.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be herein incorporated by reference. U.S. Appl. No. 62/648,174, filed Mar. 26, 2018, is incorporated herein by reference in its entirety.

1.-20. (canceled)

21. A DNA tag comprising a unique molecular identifier (UMI) and a DNA identifier, wherein the DNA tag further comprises a single-stranded portion at a 3′ end.

22. The DNA tag of claim 21, wherein the UMI and the DNA identifier are positioned in a 5′ to 3′ direction.

23. A RNA tag comprising a RNA identifier, a UMI, and a poly(T) or a template switching oligonucleotide (TSO).

24. The RNA tag of claim 23, wherein the RNA identifier, the UMI, and the poly(T) are positioned in a 5′ to 3′ direction.

25. (canceled)

26. The RNA tag of claim 23, wherein the RNA identifier, the UMI, and the TSO are positioned in a 5′ to 3′ direction.

27.-29. (canceled)

30. A composition comprising a DNA tag comprising a unique molecular identifier (UMI) and a DNA identifier, and a RNA comprising a RNA identifier, a UMI, and a poly(T).

31. The composition of claim 30, wherein the DNA tag comprises the UMI and the DNA identifier positioned in a 5′ to 3′ direction.

32. The composition of claim 30, wherein the RNA tag comprises the RNA identifier, the UMI, and the poly(T) positioned in a 5′ to 3′ position.

33. The composition of claim 30, further comprising a RNA tag comprising a RNA identifier, a UMI, and a template switching oligonucleotide (TSO).

34. The composition of claim 30, further comprising a set of gene specific primers.

35. The composition of claim 30, further comprising primers specific for the DNA tag.

36. The composition of claim 30, further comprising primers specific for the RNA tag.

37. The composition of claim 30, wherein the DNA tag further comprises a single-stranded portion at a 3′ end.
